(19) United States
(12) Patent Application Publication
(10) Pub. No.: US 2025/0313897 A1
(43) Pub. Date: Oct. 9, 2025

(54) METHODS OF ASSESSING RISK OF DEVELOPING PROSTATE CANCER

(71) Applicant: Genetic Technologies Limited, Fitzroy, Victoria (AU)

(72) Inventors: Gillian Sue DITE, Thornbury, Victoria (AU); Richard ALLMAN, Manor Lakes, Victoria (AU)

(73) Assignee: Genetic Technologies Limited, Fitzroy, Victoria (AU)

(21) Appl. No.: 18/860,560

(22) PCT Filed: Apr. 26, 2023
(86) PCT No.: PCT/AU2023/050336

§ 371 Date: Oct. 25, 2024

(30) Foreign Application Priority Data
Apr. 27, 2022 (AU) 2022901108

(51) Int. Cl. C12Q 1/6886; A61K 45/06; G16H 50/30

(52) U.S. Cl. CPC C12Q 1/6886; A61K 45/06; G16H 50/30; C12Q 2600/118; C12Q 2600/156

(57) ABSTRACT

The present disclosure relates to methods and systems for assessing the risk of a human male subject for developing prostate cancer.

### FIELD OF THE INVENTION

The present disclosure relates to methods and systems for assessing the risk of a human male subject for developing prostate cancer.

### BACKGROUND OF THE INVENTION

Prostate cancer is the most commonly diagnosed cancer in men, excluding melanoma, (Rawla, 2019) with current screening options including digital rectal examination and the prostate-specific antigen (PSA) test. PSA is an important biomarker, and while PSA screening has drastically reduced the incidence of late-stage metastatic prostate cancer, it has also caused an increase in indolent tumour detection. Men with indolent prostate cancers would likely die from a cause other than prostate cancer (Jahn et al., 2015). Thus, detection of these tumours is considered over-diagnosis. To put this into context, the lifetime risk of developing prostate cancer is approximately 13.5%, whereas the risk of dying from prostate cancer is approximately 2.5% (Barry and Simmons, 2017). Optimising prostate cancer screening by enabling detection of aggressive cancer while avoiding over-diagnosis is an important clinical objective.

Risk assessment may enable identification of men who are at substantially increased risk of developing prostate cancer. A variety of risk assessment tools are currently used in the prostate cancer space; however, most of these are used after screening to help in decision-making regarding biopsy or treatment. Few of these tools are used before screening where there is an opportunity to focus screening strategies to only include those men who are at increased risk. Such an approach has the potential to reduce over-diagnosis of prostate cancer and subsequent over-treatment (Pereira-Azevedo et al., 2017).

Because of the challenges related to over-diagnosis, routine PSA screening is a controversial subject in the international medical community. In the United States, PSA screening is recommended starting at age 50-55 years, but can begin as early as age 40 years based on clinician recommendation for high-risk men; screening frequency depends on PSA level (American Cancer Society, 2022; Fenton et al., 2018). In the United Kingdom and Australia, there is an informed choice program instead of a national PSA screening program; any man aged 50 years or older can choose to have his PSA level tested, but can begin as early as age 40 based on family history of disease (National Health Service, 2022; Public Health England, 2022; Cabarkapa et al., 2016). High risk of prostate cancer is currently determined by age, family history and ethnicity. But, there are other factors that influence risk.

Family history is a widely accepted risk factor for prostate cancer. Familial risk is strong, representing a 2- to 3-fold increased risk based on a first-degree family history of prostate cancer (Brandão et al., 2020). Some of the familial risk can be explained by high-penetrance genetic variants such as mutations in the BRCA1 and BRCA2 genes (Pilie et al., 2016). However, like with many cancers, this high-penetrance genetic susceptibility represents less than 20% of all prostate cancer cases (Brandão et al., 2020). Interestingly, there remains a genetic susceptibility in the form of low-penetrance single-nucleotide polymorphisms (SNPs) that explain a portion of risk and are largely independent of familial history risk (Conti et al., 2021). This genetic susceptibility component is known as polygenic risk.

Despite available tools, there is a need for further prostate cancer risk assessment methods.

### SUMMARY OF THE INVENTION

The present inventors have identified improved methods of assessing the risk of a human male subject for developing prostate cancer.

In one aspect, the present invention provides a method for assessing the risk of a human male subject for developing prostate cancer comprising:
i) performing a genetic risk assessment of the subject, wherein the genetic risk assessment involves detecting, in a biological sample derived from the subject, the presence of at least two polymorphisms associated with a risk of a human male subject for developing prostate cancer,
ii) performing a clinical risk assessment of the subject for developing prostate cancer, and
iii) combining the genetic risk assessment and the clinical risk assessment to obtain the risk of a human subject for developing prostate cancer.

In an embodiment, the genetic risk assessment comprises detecting the presence of at least one, at least two, at least five, at least 10, at least 25, at least 50, at least 100, at least 150, at least 200, or at least 250 of the polymorphisms selected from any one of Tables 1 to 4, or a polymorphism in linkage disequilibrium with one or more thereof.

In an embodiment, the genetic risk assessment comprises detecting the presence of each of the polymorphisms provided in Table 1, Table 2, Table 3 or Table 4, or a polymorphism in linkage disequilibrium with one or more thereof.

In an embodiment, performing the clinical risk assessment involves obtaining information from the subject on one or more of the following: age, ethnicity, family history of prostate cancer incorporating number of first-degree relatives who have had prostate cancer, the age of the youngest first-degree relative at diagnosis with prostate cancer, the number of second-degree relatives who have had prostate cancer, country of residence or protein marker levels.

In an embodiment, performing the clinical risk assessment comprises, or consists of, obtaining information from the subject on age and first-degree relative history of prostate cancer.

In an embodiment, the subject is at least 40 years old. In an embodiment, the subject is between 40 and 69 years old.

In an embodiment, the results of the risk assessment indicate that the subject should be enrolled in a screening program or subjected to more frequent screening.

In an embodiment, the polymorphism in linkage disequilibrium has linkage disequilibrium above 0.9. In an embodiment, the polymorphism in linkage disequilibrium has linkage disequilibrium of 1.

In an embodiment, the method further comprises comparing the risk to a pre-determined threshold.

In an embodiment, the genetic risk assessment produces a polygenic risk score (PRS).

In an embodiment, the polygenic risk score is determined using an odds ratio (OR) for each effect allele and effect allele frequency (p).

In an embodiment, for each polymorphism the unscaled population average risk (μ) is calculated as:

$$μ = {( 1 - p )}^{2} + 2 p ( 1 - p ) OR + {p}^{2} {OR}^{2} .$$

In an embodiment, an adjusted risk for each polymorphism is calculated as adjusted risk=(OR^{N})/μ, where N is the number of effect alleles.

In an embodiment, the polygenic risk score is determined by combining the adjusted risk for each polymorphism.

In an embodiment, the adjusted risk for each polymorphism are combined by multiplication.

In an embodiment, the method comprises determining the natural logarithm of the PRS (lnPRS).

In an embodiment, lnPRS is multiplied by a predetermined β coefficient.

In an embodiment, the β coefficient is about 1.7 such as 1.766.

In an embodiment, the method comprises determining the relative risk (rrisk) of developing prostate cancer using;

$$rrisk = {e}^{\begin{matrix}( PDCE 1 \times lnprs + PDCE 2 \times fh 1 + PDCE 3 \times fh 2 + PDCE 4 \times agegp + \\ PDCE 5 \times age \times lnprs + P DCE 6 \times age \times fh 1 + PDCE 7 \times age \times fh 2 )\end{matrix}}$$

PDCE1 is a predetermined β coefficient for the natural logarithm of a PRS,
PDCE2 is a predetermined β coefficient if the subject has at least one first-degree relative with, or who has had, prostate cancer,
PDCE3 is a predetermined β coefficient if the subject has two or more first-degree relatives with, or who have had, prostate cancer,
PDCE4 is a predetermined β coefficient for age category,
PDCE5 is a predetermined β coefficient for the interaction between age in years and the PRS,
PDCE6 is a predetermined β coefficient based on the interaction between age in years and if the subject has at least one first-degree relative with, or who has had, prostate cancer,
PDCE7 is a predetermined β coefficient based on the interaction between age in years and if the subject has two or more first-degree relatives with, or who have had, prostate cancer,
lnprs is the natural logarithm of the PRS,
fh1 is 1 if the subject has at least one first-degree relative with, or who has had, prostate cancer, and 0 if not,
fh2 is 1 if the subject has two or more first-degree relatives with, or who have had, prostate cancer, and 0 if not,
age is the age of the subject in years.

In an embodiment, the age categories are 40 to 49, 50 to 59 and 60 to 69. In an embodiment,
i) if the subject is 40 to 49 years of age the value is 1,
ii) if the subject is 50 to 59 years of age the value is 2, and
iii) if the subject is 60 to 69 years of age the value is 3.

In an embodiment, one or more or all of the following apply;
a) PDCE1 is between 1.266 and 2.266,
b) PDCE2 is between 1.716 and 2.716,
c) PDCE3 is between 4.084 and 6.084,
d) PDCE4 is between 0.022 and 0.082,
e) PDCE5 is between −0.003 and −0.023,
f) PDCE6 is between −0.017 and −0.037,
f) PDCE7 is between −0.035 and −0.095.

In an embodiment, one or more or all of the following apply;
a) PDCE1 is 1.766,
b) PDCE2 is 2.216,
c) PDCE3 is 5.084,
d) PDCE4 is 0.052,
e) PDCE5 is −0.013,
f) PDCE6 is −0.027,
f) PDCE7 is −0.065.

In an alternate embodiment, the β coefficient is about 1.03 such as 1.0307.

In an alternate embodiment, the method comprises determining the relative risk (rrisk) of developing prostate cancer using;

$$rrisk = {e}^{\begin{matrix}( PDCE 1 \times lnprs + PDCE 2 \times fh 1 + PDCE 3 \times fh 2 + PDCE 4 \times \\ age 1 + P DCE 5 \times age 2 + P DCE 6 \times ( age - 55 ) \times lnprs )\end{matrix}}$$

where;
PDCE1 is a predetermined β coefficient for the natural logarithm of a PRS,
PDCE2 is a predetermined β coefficient if the subject has at least one first-degree relative with, or who has had, prostate cancer,
PDCE3 is a predetermined β coefficient if the subject has two or more first-degree relatives with, or who have had, prostate cancer,
PDCE4 is a predetermined β coefficient if the subject is 50 to 59 years of age,
PDCE5 is a predetermined β coefficient if the subject is 60 to 69 years of age,
PDCE6 is a predetermined β coefficient based on an interaction between the age of the subject and the natural logarithm of the PRS,
lnprs is the natural logarithm of the PRS,
fh1 is 1 if the subject has at least one first-degree relative with, or who has had, prostate cancer, and 0 if not,
fh2 is 1 if the subject has two or more first-degree relatives with, or who have had, prostate cancer, and 0 if not,
age1 is 1 if the subject is 50 to 59 years of age and 0 if not,
age2 is 1 if the subject is 60 to 69 years of age and 0 if not,
age is the age of the subject in years.

In an embodiment, one or more or all of the following apply;
a) PDCE1 is between 0.913 and 1.113,
b) PDCE2 is between 0.327 and 0.527,
c) PDCE3 is between 0.737 and 0.937,
d) PDCE4 is between 0.023 and 0.223,
e) PDCE5 is between 0.177 and 0.377,
f) PDCE6 is between −0.089 and 0.111.

In an embodiment, one or more or all of the following apply;
a) PDCE1 is 1.013,
b) PDCE2 is 0.427,
c) PDCE3 is 0.837,
d) PDCE4 is 0.123,
e) PDCE5 is 0.277,
f) PDCE6 is 0.011.

In an embodiment, the method comprises determining one or more or all of the absolute 5-year risk, the absolute 10-year risk, or the absolute remaining lifetime risk (to age 90).

In another aspect, the present invention provides a computer-implemented method for assessing the risk of a human male subject for developing prostate cancer, the method operable in a computing system comprising a processor and a memory, the method comprising:
receiving clinical risk data and genetic risk data for the male subject, wherein the clinical and genetic risk data was obtained by a method of the invention;
processing the data to combine the clinical risk data with the genetic risk data to obtain the risk of a human male subject for developing prostate cancer;
outputting the risk of a human male subject for developing prostate cancer.

In an embodiment, the clinical risk data and genetic risk data for the subject is received from a user interface coupled to the computing system.

In an embodiment, the clinical risk data and genetic risk data for the subject is received from a remote device across a wireless communications network.

In an embodiment, outputting comprises outputting information to a user interface coupled to the computing system.

In an embodiment, the method comprises determining a genetic risk score based on genetic data derived from a biological sample taken from the male subject.

In an embodiment, the method comprises determining the relative risk (rrisk) of developing prostate cancer using;

$$rrisk = {e}^{\begin{matrix}( PDCE 1 \times lnprs + PDCE 2 \times fh 1 + PDCE 3 \times fh 2 + PDCE 4 \times agegp + \\ PDCE 5 \times age \times lnprs + P DCE 6 \times age \times fh 1 + PDCE 7 \times age \times fh 2 )\end{matrix}}$$

PDCE1 is a predetermined β coefficient for the natural logarithm of a PRS,
PDCE2 is a predetermined β coefficient if the subject has at least one first-degree relative with, or who has had, prostate cancer,
PDCE3 is a predetermined β coefficient if the subject has two or more first-degree relatives with, or who have had, prostate cancer,
PDCE4 is a predetermined β coefficient for age category,
PDCE5 is a predetermined β coefficient for the interaction between age in years and the PRS,
PDCE6 is a predetermined β coefficient based on the interaction between age in years and if the subject has at least one first-degree relative with, or who has had, prostate cancer,
PDCE7 is a predetermined β coefficient based on the interaction between age in years and if the subject has two or more first-degree relatives with, or who have had, prostate cancer,
lnprs is the natural logarithm of the PRS,
fh1 is 1 if the subject has at least one first-degree relative with, or who has had, prostate cancer, and 0 if not,
fh2 is 1 if the subject has two or more first-degree relatives with, or who have had, prostate cancer, and 0 if not,
age is the age of the subject in years.

In an alternate embodiment, the method comprises determining the relative risk (rrisk) of developing prostate cancer using;

$$rrisk = {e}^{( P DCE 1 \times lnps + PDCE 2 \times fh 1 + PDCE 3 \times fh 2 + PDCE 4 \times age 1 + P DCE 5 \times age 2 + P DCE 6 \times ( age - 55 ) \times lnp r s}$$

where;
PDCE1 is a predetermined β coefficient for the natural logarithm of a PRS,
PDCE2 is a predetermined β coefficient if the subject has at least one first-degree relative with, or who has had, prostate cancer,
PDCE3 is a predetermined β coefficient if the subject has two or more first-degree relatives with, or who have had, prostate cancer,
PDCE4 is a predetermined β coefficient if the subject is 50 to 59 years of age,
PDCE5 is a predetermined β coefficient if the subject is 60 to 69 years of age,
PDCE6 is a predetermined β coefficient based on an interaction between the age of the subject and the natural logarithm of the PRS,
lnprs is the natural logarithm of the PRS, fh1 is 1 if the subject has at least one first degree relative with, or who have had, prostate cancer, and 0 if not,
fh2 is 1 if the subject has two or more first degree relative with, or who have had, prostate cancer, and 0 if not,
age1 is 1 if the subject is 50 to 59 years of age and 0 if not,
age2 is 1 if the subject is 60 to 69 years of age and 0 if not, age is the age of the subject in years.

In a further aspect, the present invention provides computer readable storage medium storing executable code, wherein when a processor executes the code, the processor is cause to perform a computer-implemented method of the invention.

Furthermore, provided is a device for assessing the risk of a human male subject developing prostate cancer, the device comprising:
a processor; and
a memory device storing executable code, the memory being accessible to the processor;
wherein when caused to execute the executable code stored in the memory device, the processor is caused to perform a method of the invention.

In an embodiment, the device further comprises a display component, wherein the processor is further caused to display the prostate cancer risk score of the male subject for developing prostate cancer on the display component.

In an embodiment, the device further comprises a communications module, wherein the processor is further caused to communicate the prostate cancer risk score of the male subject for developing prostate cancer to an external device via the communications module.

In another aspect, the present invention provides a method for determining the need for routine diagnostic testing of a human male subject for prostate cancer comprising assessing the risk of the subject for developing prostate cancer using a method of the invention.

In another aspect, the present invention provides a method of screening for prostate cancer in a human male subject, the method comprising assessing the risk of the subject for developing prostate cancer using a method of the invention, and routinely screening for prostate cancer in the subject if they are assessed as having a risk for developing prostate cancer.

In another aspect, the present invention provides a method for determining the need of a human male subject for prophylactic anti-prostate cancer therapy comprising assessing the risk of the subject for developing prostate cancer using a method of the invention.

In another aspect, the present invention provides a method for preventing prostate cancer in a human male subject, the method comprising assessing the risk of the subject for developing prostate cancer using a method of the invention, and administering an anti-prostate cancer therapy to the subject if they are assessed as having a risk for developing prostate cancer.

In another aspect, the present invention provides an anti-prostate cancer therapy for use in preventing prostate cancer in a human male subject at risk thereof, wherein the subject is assessed as having a risk for developing prostate cancer using a method of the invention.

In another aspect, the present invention provides a method for stratifying a group of human male subjects for a clinical trial of a candidate therapy, the method comprising assessing the individual risk of the subjects for developing prostate cancer using a method of the invention, and using the results of the assessment to select subjects more likely to be responsive to the therapy.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1. Standardised incidence ratios of the observed number of prostate cancers in the full dataset compared with the number expected using population incidence rates by decile of 5-year risk pf prostate cancer.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., oncology, prostate cancer analysis, molecular genetics, biostatistics, risk assessment and clinical studies).

Unless otherwise indicated, the molecular, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

It is to be understood that this disclosure is not limited to particular embodiments, which can, of course, vary. It is also to be understood that the terminology used herein is for the purpose of describing particular embodiments only, and is not intended to be limiting. As used in this specification and the appended claims, terms in the singular and the singular forms “a,” “an” and “the,” for example, optionally include plural referents unless the content clearly dictates otherwise. Thus, for example, reference to “a probe” optionally includes a plurality of probe molecules; similarly, depending on the context, use of the term “a nucleic acid” optionally includes, as a practical matter, many copies of that nucleic acid molecule.

As used herein, the term “about”, unless stated to the contrary, refers to ±10%, more preferably ±5%, more preferably ±1%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The methods of the present disclosure can be used to assess risk of a human male subject developing prostate cancer. As used herein, the term “prostate cancer” encompasses any type of prostate cancer that can develop in a male subject. For example, the prostate cancer may be acinar adenocarcinoma, ductal adenocarcinoma, transitional cell (urothelial) cancer, squamous cell cancer or small cell prostate cancer.

As used herein, “biological sample” refers to any sample comprising nucleic acids, especially DNA, from or derived from a human patient, e.g., bodily fluids (blood, saliva, urine etc.), biopsy, tissue, and/or waste from the patient. Thus, tissue biopsies, stool, sputum, saliva, blood, lymph, or the like can easily be screened for polymorphisms, as can essentially any tissue of interest that contains the appropriate nucleic acids. In one embodiment, the biological sample is a cheek cell sample. These samples are typically taken, following informed consent, from a patient by standard medical laboratory methods. The sample may be in a form taken directly from the patient, or may be at least partially processed (purified) to remove at least some non-nucleic acid material.

A “polymorphism” is a locus that is variable; that is, within a population, the nucleotide sequence at a polymorphism has more than one version or allele. One example of a polymorphism is a “single-nucleotide polymorphism”, which is a polymorphism at a single-nucleotide position in a genome (the nucleotide at the specified position varies between individuals or populations). Other examples include a deletion or insertion of one or more base pairs at the polymorphism locus.

As used herein, the term “SNP” or “single-nucleotide polymorphism” refers to a genetic variation between individuals; e.g., a single nitrogenous base position in the DNA of organisms that is variable. As used herein, “SNPs” is the plural of SNP. Of course, when one refers to DNA herein, such reference may include derivatives of the DNA such as amplicons, RNA transcripts thereof, etc.

The term “allele” refers to one of two or more different nucleotide sequences that occur or are encoded at a specific locus, or two or more different polypeptide sequences encoded by such a locus. For example, a first allele can occur on one chromosome, while a second allele occurs on a second homologous chromosome, e.g., as occurs for different chromosomes of a heterozygous individual, or between different homozygous or heterozygous individuals in a population. An allele “positively” correlates with a trait when it is linked to it and when presence of the allele is an indicator that the trait or trait form will occur in an individual comprising the allele. An allele “negatively” correlates with a trait when it is linked to it and when presence of the allele is an indicator that a trait or trait form will not occur in an individual comprising the allele.

A marker polymorphism or allele is “correlated”, or “associated” with a specified phenotype (prostate cancer susceptibility, etc.) when it can be statistically linked (positively or negatively) to the phenotype (also referred to herein as an “effect allele”). Methods for determining whether a polymorphism or allele is statistically linked are known to those in the art. That is, the specified polymorphism occurs more commonly in a case population (e.g., prostate cancer patients) than in a control population (e.g., individuals that do not have prostate cancer). This correlation is often inferred as being causal in nature, but it need not be, simple genetic linkage to (association with) a locus for a trait that underlies the phenotype is sufficient for correlation/association to occur.

The phrase “linkage disequilibrium” (LD) is used to describe the statistical correlation between two neighbouring polymorphic genotypes. Typically, LD refers to the correlation between the alleles of a random gamete at the two loci, assuming Hardy-Weinberg equilibrium (statistical independence) between gametes. LD is quantified with either Lewontin's parameter of association (D′) or with Pearson correlation coefficient (r) (Devlin and Risch, 1995). Two loci with a LD value of 1 are said to be in complete LD. At the other extreme, two loci with a LD value of 0 are termed to be in linkage equilibrium. Linkage disequilibrium is calculated following the application of the expectation maximization algorithm for the estimation of haplotype frequencies (Slatkin and Excoffier, 1996). LD values according to the present disclosure for neighbouring genotypes/loci are selected above 0.1, preferably, above 0.2, more preferable above 0.5, more preferably, above 0.6, still more preferably, above 0.7, preferably, above 0.8, more preferably above 0.9, ideally about 1.0.

Another way one of skill in the art can readily identify polymorphisms in linkage disequilibrium with the polymorphisms of the present disclosure is determining the LOD score for two loci. LOD stands for “logarithm of the odds”, a statistical estimate of whether two genes, or a gene and a disease gene, are likely to be located near each other on a chromosome and are therefore likely to be inherited. A LOD score of between about 2-3 or higher is generally understood to mean that two genes are located close to each other on the chromosome. The present inventors have found that many of the polymorphisms in linkage disequilibrium with the polymorphisms of the present disclosure have a LOD score of between about 2-50. Accordingly, in an embodiment, LOD values according to the present disclosure for neighbouring genotypes/loci are selected at least above 2, at least above 3, at least above 4, at least above 5, at least above 6, at least above 7, at least above 8, at least above 9, at least above 10, at least above 20 at least above 30, at least above 40, at least above 50.

In another embodiment, polymorphisms in linkage disequilibrium with the polymorphisms of the present disclosure can have a specified genetic recombination distance of less than or equal to about 20 centimorgan (cM) or less. For example, 15 CM or less, 10 cM or less, 9 cM or less, 8 CM or less, 7 CM or less, 6 CM or less, 5 CM or less, 4 cM or less, 3 cM or less, 2 cM or less, 1 cM or less, 0.75 cM or less, 0.5 CM or less, 0.25 cM or less, or 0.1 cM or less. For example, two linked loci within a single chromosome segment can undergo recombination during meiosis with each other at a frequency of less than or equal to about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, about 1%, about 0.75%, about 0.5%, about 0.25%, or about 0.1% or less.

In another embodiment, polymorphisms in linkage disequilibrium with the polymorphisms of the present disclosure are within at least 100 kb (which correlates in humans to about 0.1 cM, depending on local recombination rate), at least 50 kb, at least 20 kb or less of each other.

For example, one approach for the identification of surrogate markers for a particular polymorphism involves a simple strategy that presumes that polymorphisms surrounding the target polymorphism are in linkage disequilibrium and can therefore provide information about disease susceptibility. Thus, as described herein, surrogate markers can therefore be identified from publicly available databases, such as HAPMAP, by searching for polymorphisms fulfilling certain criteria which have been found in the scientific community to be suitable for the selection of surrogate marker candidates.

“Allele frequency”, or number of a particular allele, refers to the frequency (proportion or percentage) at which an allele is present at a locus within an individual, within a line or within a population of lines. For example, for an allele “A,” diploid individuals of genotype “AA”, “Aa” or “aa” (alternatively “AA”, “AB” or “BB”) have allele frequencies of 1.0, 0.5, or 0.0, respectively. One can estimate the allele frequency within a line or population (e.g., cases or controls) by averaging the allele frequencies of a sample of individuals from that line or population. Similarly, one can calculate the allele frequency within a population of lines by averaging the allele frequencies of lines that make up the population.

In an embodiment, the term “allele frequency” is used to define the population frequency of the allele of interest, which is known as the effect allele. The effect allele is that linked to prostate cancer risk, either positively or negatively.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

A “locus” is a chromosomal position or region. For example, a polymorphic locus is a position or region where a polymorphic nucleic acid, trait determinant, gene or marker is located. In a further example, a “gene locus” is a specific chromosome location (region) in the genome of a species where a specific gene can be found.

A “marker”, “molecular marker” or “marker nucleic acid” refers to a nucleotide sequence or encoded product thereof (e.g., a protein) used as a point of reference when identifying a locus or a linked locus. A marker can be derived from genomic nucleotide sequence or from expressed nucleotide sequences (e.g., from an RNA, nRNA, mRNA, a cDNA, etc.), or from an encoded polypeptide. The term also refers to nucleic acid sequences complementary to or flanking the marker sequences, such as nucleic acids used as probes or primer pairs capable of amplifying the marker sequence. A “marker probe” is a nucleic acid sequence or molecule that can be used to identify the presence of a marker locus, e.g., a nucleic acid probe that is complementary to a marker locus sequence. Nucleic acids are “complementary” when they specifically hybridize in solution, e.g., according to Watson-Crick base pairing rules. A “marker locus” is a locus that can be used to track the presence of a second linked locus, e.g., a linked or correlated locus that encodes or contributes to the population variation of a phenotypic trait. For example, a marker locus can be used to monitor segregation of alleles at a locus, such as a QTL, that are genetically or physically linked to the marker locus. Thus, a “marker allele,” alternatively an “allele of a marker locus” is one of a plurality of polymorphic nucleotide sequences found at a marker locus in a population that is polymorphic for the marker locus. Each of the identified markers is expected to be in close physical and genetic proximity (resulting in physical and/or genetic linkage) to a genetic element, e.g., a QTL, that contributes to the relevant phenotype. Markers corresponding to genetic polymorphisms between members of a population can be detected by methods well-established in the art. These include, e.g., DNA sequencing, PCR-based sequence specific amplification methods, detection of restriction fragment length polymorphisms (RFLP), detection of isozyme markers, detection of allele specific hybridization (ASH), detection of single-nucleotide extension, detection of amplified variable sequences of the genome, detection of self-sustained sequence replication, detection of simple sequence repeats (SSRs), detection of single-nucleotide polymorphisms (SNPs), or detection of amplified fragment length polymorphisms (AFLPs).

The term “amplifying” in the context of nucleic acid amplification is any process whereby additional copies of a selected nucleic acid (or a transcribed form thereof) are produced. Typical amplification methods include various polymerase based replication methods, including the polymerase chain reaction (PCR), ligase mediated methods such as the ligase chain reaction (LCR) and RNA polymerase based amplification (e.g., by transcription) methods.

An “amplicon” is an amplified nucleic acid, e.g., a nucleic acid that is produced by amplifying a template nucleic acid by any available amplification method (e.g., PCR, LCR, transcription, or the like).

A “gene” is one or more sequence(s) of nucleotides in a genome that together encode one or more expressed molecules, e.g., an RNA, or polypeptide. The gene can include coding sequences that are transcribed into RNA which may then be translated into a polypeptide sequence, and can include associated structural or regulatory sequences that aid in replication or expression of the gene.

A “genotype” is the genetic constitution of an individual (or group of individuals) at one or more genetic loci. Genotype is defined by the allele(s) of one or more known loci of the individual, typically, the compilation of alleles inherited from its parents.

A “haplotype” is the genotype of an individual at a plurality of genetic loci on a single DNA strand. Typically, the genetic loci described by a haplotype are physically and genetically linked, i.e., on the same chromosome strand.

A “set” of markers, probes or primers refers to a collection or group of markers probes, primers, or the data derived therefrom, used for a common purpose, e.g., identifying an individual with a specified genotype (e.g., risk of developing prostate cancer). Frequently, data corresponding to the markers, probes or primers, or derived from their use, is stored in an electronic medium. While each of the members of a set possess utility with respect to the specified purpose, individual markers selected from the set as well as subsets including some, but not all of the markers, are also effective in achieving the specified purpose.

The polymorphisms and genes, and corresponding marker probes, amplicons or primers described above can be embodied in any system herein, either in the form of physical nucleic acids, or in the form of system instructions that include sequence information for the nucleic acids. For example, the system can include primers or amplicons corresponding to (or that amplify a portion of) a gene or polymorphism described herein. As in the methods above, the set of marker probes or primers optionally detects a plurality of polymorphisms in a plurality of said genes or genetic loci. Thus, for example, the set of marker probes or primers detects at least one polymorphism in each of these polymorphisms or genes, or any other polymorphism, gene or locus defined herein. Any such probe or primer can include a nucleotide sequence of any such polymorphism or gene, or a complementary nucleic acid thereof, or a transcribed product thereof (e.g., a nRNA or mRNA form produced from a genomic sequence, e.g., by transcription or splicing).

As used herein, “risk assessment” refers to a process by which a subject's risk of developing prostate cancer can be assessed. A risk assessment will typically involve obtaining information relevant to the subject's risk of developing prostate cancer, assessing that information, and quantifying the subject's risk of developing prostate cancer, for example, by producing a risk score.

As used herein, the terms “routinely screening for prostate cancer” and “more frequent screening” are relative terms, and are based on a comparison to the level of screening recommended to a subject who has not identified risk of developing prostate cancer. Skilled clinicians can readily determine suitable frequencies based on their knowledge of the field. Examples of prostate screening methods include, but are not limited to, digital rectal examination and the protein marker detection such as a prostate-specific antigen (PSA) test, magnetic resonance imaging (MRI), MiCheck (Shore et al., 2020) and transrectal ultrasound.

### Clinical Risk Factors

Clinical information can be self-reported by the male subject. For example, the subject may complete a questionnaire designed to obtain clinical information regarding the clinical factors. In another example, subject to obtaining informed consent from the male subject, clinical information can be obtained from medical records by interrogating a relevant database comprising the clinical information.

In an embodiment, the clinical risk assessment involves obtaining information from the subject on one or more of the following: age, ethnicity, family history of prostate cancer incorporating number of first-degree relatives who have had prostate cancer, the age of the youngest first-degree relative at diagnosis with prostate cancer, the number of second-degree relatives who have had prostate cancer, country of residence or protein marker levels.

An example of a protein marker is prostate-specific antigen (PSA).

In an embodiment, the clinical risk assessment comprises, or preferably consists of, obtaining information from the subject on age and first-degree relative history of prostate cancer.

“Family history of prostate cancer” or variations thereof is used in the context of the present disclosure to refer to the history of prostate cancer amongst the male subject's first- and/or second-degree relatives. For example, “family history of prostate cancer” can be used to refer to the history of prostate cancer amongst only first-degree relatives. Put another way, the clinical risk assessment procedure can take into consideration the subject's family history of prostate cancer amongst first-degree relatives. In the context of the present disclosure, a “first-degree relative” is a family member who shares about 50 percent of their genes with the male subject. Examples of first-degree relatives include parents, offspring, and full-siblings. A “second-degree relative” is a family member who shares about 25 percent of their genes with the subject. Examples of second-degree relatives include uncles, aunts, nephews, nieces, grandparents, grandchildren, and half-siblings.

In another embodiment, the male subject's family history of prostate cancer is based on the male subject's first degree-relatives and second degree relatives.

In another embodiment, the male subject's family history of prostate cancer is based on the male subject's first degree-relatives.

As used herein, “based on” means that values are assigned to, for example, the subject's age and family history of prostate cancer, but then any suitable calculations are conducted to determine clinical risk or risk based on genetic and clinical factors.

In an embodiment, the subject is between 40 and 69 years of age.

In an embodiment, the subject does not have, or has not had, prostate cancer.

In another embodiment, performing the clinical risk assessment uses a model which calculates the absolute risk of developing prostate cancer. For example, the absolute risk of developing prostate cancer can be calculated using cancer incidence rates.

### Genetic Risk Factors

Various exemplary polymorphisms associated with prostate cancer are discussed in the present disclosure. These polymorphisms vary in terms of penetrance and many would be understood by those of skill in the art to be low penetrance polymorphisms.

The term “penetrance” is used in the context of the present disclosure to refer to the frequency at which a particular polymorphism manifests itself within male subjects with prostate cancer. “High penetrance” polymorphisms will often be apparent in a male subject with prostate cancer (such as those with an odds ratio greater than 1.5 or greater than 2) while “low penetrance” polymorphisms will only sometimes be apparent cancer (such as those with an odds ratio less than 1.5). In an embodiment polymorphisms assessed as part of a genetic risk assessment according to the present disclosure are low penetrance polymorphisms.

In an embodiment, the genetic risk assessment is performed by analysing the genotype of the subject at 25 or more loci for polymorphisms associated with prostate cancer. In an embodiment, the genetic risk assessment is performed by analysing the genotype of the subject at 50 or more loci for polymorphisms associated with prostate cancer. In an embodiment, the genetic risk assessment is performed by analysing the genotype of the subject at 100 or more loci for polymorphisms associated with prostate cancer. In an embodiment, the genetic risk assessment is performed by analysing the genotype of the subject at 150 or more loci for polymorphisms associated with prostate cancer. In an embodiment, the genetic risk assessment is performed by analysing the genotype of the subject at 200 or more loci for polymorphisms associated with prostate cancer. In an embodiment, the genetic risk assessment is performed by analysing the genotype of the subject at 250 or more loci for polymorphisms associated with prostate cancer.

In an embodiment, the genetic risk assessment is performed by analysing the genotype of the subject at 25 or more loci for polymorphisms provided in Tables 1 to 4, or a polymorphism in linkage disequilibrium with one or more thereof. In an embodiment, the genetic risk assessment is performed by analysing the genotype of the subject at 50 or more loci for polymorphisms provided in Tables 1 to 4, or a polymorphism in linkage disequilibrium with one or more thereof. In an embodiment, the genetic risk assessment is performed by analysing the genotype of the subject at 100 or more loci for polymorphisms provided in Tables 1 to 4, or a polymorphism in linkage disequilibrium with one or more thereof. In an embodiment, the genetic risk assessment is performed by analysing the genotype of the subject at 150 or more loci for polymorphisms provided in Tables 1 to 4, or a polymorphism in linkage disequilibrium with one or more thereof. In an embodiment, the genetic risk assessment is performed by analysing the genotype of the subject at 200 or more loci for polymorphisms provided in Tables 1 to 4, or a polymorphism in linkage disequilibrium with one or more thereof. In an embodiment, the genetic risk assessment is performed by analysing the genotype of the subject at 250 or more loci for polymorphisms provided in Tables 1 to 4, or a polymorphism in linkage disequilibrium with one or more thereof. In an embodiment, the genetic risk assessment is performed by analysing the genotype of the subject at each polymorphism provided in Tables 1 to 4, or a polymorphism in linkage disequilibrium with one or more thereof.

In an embodiment, if the subject is Caucasian, such as self-reported Caucasian, the genetic risk assessment is performed by analysing the genotype of the subject at 25 or more loci for polymorphisms provided in Table 1, or a polymorphism in linkage disequilibrium with one or more thereof. In an embodiment, if the subject is Caucasian, such as self-reported Caucasian, the genetic risk assessment is performed by analysing the genotype of the subject at 50 or more loci for polymorphisms provided in Table 1, or a polymorphism in linkage disequilibrium with one or more thereof. In an embodiment, if the subject is Caucasian, such as self-reported Caucasian, the genetic risk assessment is performed by analysing the genotype of the subject at 100 or more loci for polymorphisms provided in Table 1, or a polymorphism in linkage disequilibrium with one or more thereof. In an embodiment, if the subject is Caucasian, such as self-reported Caucasian, the genetic risk assessment is performed by analysing the genotype of the subject at 150 or more loci for polymorphisms provided in Table 1, or a polymorphism in linkage disequilibrium with one or more thereof. In an embodiment, if the subject is Caucasian, such as self-reported Caucasian, the genetic risk assessment is performed by analysing the genotype of the subject at 200 or more loci for polymorphisms provided in Table 1, or a polymorphism in linkage disequilibrium with one or more thereof. In an embodiment, if the subject is Caucasian, such as self-reported Caucasian, the genetic risk assessment is performed by analysing the genotype of the subject at 250 or more loci for polymorphisms provided in Table 1, or a polymorphism in linkage disequilibrium with one or more thereof. In an embodiment, if the subject is Caucasian, such as self-reported Caucasian, the genetic risk assessment is performed by analysing the genotype of the subject at each polymorphism provided in Table 1, or a polymorphism in linkage disequilibrium with one or more thereof.

In an embodiment, if the subject is African, such as self-reported African, the genetic risk assessment is performed by analysing the genotype of the subject at 25 or more loci for polymorphisms provided in Table 2, or a polymorphism in linkage disequilibrium with one or more thereof. In an embodiment, if the subject is African, such as self-reported African, the genetic risk assessment is performed by analysing the genotype of the subject at 50 or more loci for polymorphisms provided in Table 2, or a polymorphism in linkage disequilibrium with one or more thereof. In an embodiment, if the subject is African, such as self-reported African, the genetic risk assessment is performed by analysing the genotype of the subject at 100 or more loci for polymorphisms provided in Table 2, or a polymorphism in linkage disequilibrium with one or more thereof. In an embodiment, if the subject is African, such as self-reported African, the genetic risk assessment is performed by analysing the genotype of the subject at 150 or more loci for polymorphisms provided in Table 2, or a polymorphism in linkage disequilibrium with one or more thereof. In an embodiment, if the subject is African, such as self-reported African, the genetic risk assessment is performed by analysing the genotype of the subject at 200 or more loci for polymorphisms provided in Table 2, or a polymorphism in linkage disequilibrium with one or more thereof. In an embodiment, if the subject is African, such as self-reported African, the genetic risk assessment is performed by analysing the genotype of the subject at 250 or more loci for polymorphisms provided in Table 2, or a polymorphism in linkage disequilibrium with one or more thereof. In an embodiment, if the subject is African, such as self-reported African, the genetic risk assessment is performed by analysing the genotype of the subject at each polymorphism provided in Table 2, or a polymorphism in linkage disequilibrium with one or more thereof.

In an embodiment, if the subject is East Asian, such as self-reported East Asian, the genetic risk assessment is performed by analysing the genotype of the subject at 25 or more loci for polymorphisms provided in Table 3, or a polymorphism in linkage disequilibrium with one or more thereof. In an embodiment, if the subject is East Asian, such as self-reported East Asian, the genetic risk assessment is performed by analysing the genotype of the subject at 50 or more loci for polymorphisms provided in Table 3, or a polymorphism in linkage disequilibrium with one or more thereof. In an embodiment, if the subject is East Asian, such as self-reported East Asian, the genetic risk assessment is performed by analysing the genotype of the subject at 100 or more loci for polymorphisms provided in Table 3, or a polymorphism in linkage disequilibrium with one or more thereof. In an embodiment, if the subject is East Asian, such as self-reported East Asian, the genetic risk assessment is performed by analysing the genotype of the subject at 150 or more loci for polymorphisms provided in Table 3, or a polymorphism in linkage disequilibrium with one or more thereof. In an embodiment, if the subject is East Asian, such as self-reported East Asian, the genetic risk assessment is performed by analysing the genotype of the subject at 200 or more loci for polymorphisms provided in Table 3, or a polymorphism in linkage disequilibrium with one or more thereof. In an embodiment, if the subject is East Asian, such as self-reported East Asian, the genetic risk assessment is performed by analysing the genotype of the subject at 250 or more loci for polymorphisms provided in Table 3, or a polymorphism in linkage disequilibrium with one or more thereof. In an embodiment, if the subject is East Asian, such as self-reported East Asian, the genetic risk assessment is performed by analysing the genotype of the subject at each polymorphism provided in Table 3, or a polymorphism in linkage disequilibrium with one or more thereof.

In an embodiment, if the subject is Hispanic, such as self-reported Hispanic, the genetic risk assessment is performed by analysing the genotype of the subject at 25 or more loci for polymorphisms provided in Table 4, or a polymorphism in linkage disequilibrium with one or more thereof. In an embodiment, if the subject is Hispanic, such as self-reported Hispanic, the genetic risk assessment is performed by analysing the genotype of the subject at 50 or more loci for polymorphisms provided in Table 4, or a polymorphism in linkage disequilibrium with one or more thereof. In an embodiment, if the subject is Hispanic, such as self-reported Hispanic, the genetic risk assessment is performed by analysing the genotype of the subject at 100 or more loci for polymorphisms provided in Table 4, or a polymorphism in linkage disequilibrium with one or more thereof. In an embodiment, if the subject is Hispanic, such as self-reported Hispanic, the genetic risk assessment is performed by analysing the genotype of the subject at 150 or more loci for polymorphisms provided in Table 4, or a polymorphism in linkage disequilibrium with one or more thereof. In an embodiment, if the subject is Hispanic, such as self-reported Hispanic, the genetic risk assessment is performed by analysing the genotype of the subject at 200 or more loci for polymorphisms provided in Table 4, or a polymorphism in linkage disequilibrium with one or more thereof. In an embodiment, if the subject is Hispanic, such as self-reported Hispanic, the genetic risk assessment is performed by analysing the genotype of the subject at 250 or more loci for polymorphisms provided in Table 4, or a polymorphism in linkage disequilibrium with one or more thereof. In an embodiment, if the subject is Hispanic, such as self-reported Hispanic, the genetic risk assessment is performed by analysing the genotype of the subject at each polymorphism provided in Table 4, or a polymorphism in linkage disequilibrium with one or more thereof.

TABLE 1
Caucasian polymorphisms.

| | | | | | Effect | | |
|---|---|---|---|---|---|---|---|
| | | | Effect | Reference | Allele | | |
| Polymorphism | Chromosome | Position | Allele | Allele | Frequency | OR | (95% CI) |
| rs7542260 | 1 | 5743196 | T | C | 0.067 | 0.976 | (0.94-1.01) |
| rs2847344 | 1 | 10564675 | A | G | 0.692 | 1.044 | (1.03-1.06) |
| rs10803412 | 1 | 16376831 | C | T | 0.176 | 1.054 | (1.03-1.07) |
| rs544780844 | 1 | 46251655 | T | C | 0.188 | 1.06 | (1.04-1.08) |
| rs56391074 | 1 | 88210715 | AT | A | 0.370 | 1.045 | (1.03-1.06) |
| rs1811698 | 1 | 150772613 | C | T | 0.895 | 1.086 | (1.06-1.11) |
| rs607518 | 1 | 150954671 | A | G | 0.209 | 1.066 | (1.05-1.09) |
| rs10127983 | 1 | 153923276 | T | C | 0.312 | 1.068 | (1.05-1.09) |
| rs56103503 | 1 | 154980351 | T | C | 0.386 | 1.057 | (1.04-1.07) |
| rs147847496 | 1 | 155118588 | C | T | 0.968 | 1.168 | (1.12-1.22) |
| rs184104770 | 1 | 155690186 | A | C | 0.019 | 1.172 | (1.11-1.24) |
| rs80237341 | 1 | 157119915 | C | G | 0.018 | 1.138 | (1.07-1.21) |
| rs6660538 | 1 | 163295678 | A | C | 0.363 | 1.034 | (1.02-1.05) |
| rs4075646 | 1 | 167135941 | T | A | 0.043 | 1.057 | (1.02-1.10) |
| rs507603 | 1 | 179897070 | A | C | 0.159 | 1.042 | (1.02-1.06) |
| rs34295433 | 1 | 183032447 | CTAAG | C | 0.536 | 1.041 | (1.03-1.06) |
| rs138638958 | 1 | 204030362 | TTTTG | T | 0.540 | 1.046 | (1.03-1.06) |
| rs4245739 | 1 | 204518842 | A | C | 0.739 | 1.095 | (1.08-1.11) |
| rs708723 | 1 | 205739266 | C | T | 0.439 | 1.044 | (1.03-1.06) |
| rs11686272 | 2 | 8598444 | T | G | 0.440 | 1.045 | (1.03-1.06) |
| rs73913932 | 2 | 10094526 | G | A | 0.070 | 1.085 | (1.06-1.12) |
| rs1990613 | 2 | 10781975 | T | C | 0.504 | 1.069 | (1.05-1.09) |
| rs7602028 | 2 | 16016503 | C | A | 0.711 | 1.052 | (1.03-1.07) |
| rs9306894 | 2 | 20878105 | G | A | 0.363 | 1.077 | (1.06-1.09) |
| rs6738169 | 2 | 43064555 | C | G | 0.703 | 1.044 | (1.03-1.06) |
| rs7591218 | 2 | 43637998 | A | G | 0.315 | 1.069 | (1.05-1.09) |
| rs28514770 | 2 | 43851282 | C | G | 0.710 | 1.046 | (1.03-1.06) |
| rs11125927 | 2 | 62752975 | G | A | 0.111 | 1.068 | (1.04-1.09) |
| rs58235267 | 2 | 63277843 | G | C | 0.470 | 1.119 | (1.10-1.14) |
| rs139283528 | 2 | 63938756 | G | A | 0.986 | 1.276 | (1.20-1.36) |
| rs74702681 | 2 | 66652885 | T | C | 0.022 | 1.174 | (1.12-1.23) |
| rs2028900 | 2 | 85767735 | C | T | 0.558 | 1.086 | (1.07-1.10) |
| rs2165108 | 2 | 111861993 | A | T | 0.044 | 1.101 | (1.06-1.14) |
| rs11691517 | 2 | 111893096 | T | G | 0.742 | 1.06 | (1.04-1.08) |
| rs111595856 | 2 | 121103598 | T | C | 0.070 | 1.087 | (1.05-1.12) |
| rs10206072 | 2 | 121373466 | G | A | 0.891 | 1.052 | (1.03-1.08) |
| rs16854905 | 2 | 169012955 | C | T | 0.899 | 1.056 | (1.03-1.08) |
| rs77167534 | 2 | 173319930 | C | T | 0.941 | 1.269 | (1.23-1.31) |
| rs34925593 | 2 | 174234547 | C | T | 0.481 | 1.049 | (1.03-1.07) |
| rs1861270 | 2 | 202126615 | G | A | 0.733 | 1.052 | (1.03-1.07) |
| rs12621900 | 2 | 208118301 | C | T | 0.769 | 1.052 | (1.03-1.07) |
| rs74001374 | 2 | 238411293 | C | T | 0.993 | 1.13 | (1.02-1.25) |
| rs2292884 | 2 | 238443226 | G | A | 0.241 | 1.063 | (1.04-1.08) |
| rs77559646 | 2 | 242135265 | A | G | 0.023 | 1.328 | (1.26-1.40) |
| rs77482050 | 2 | 242139600 | G | A | 0.987 | 1.432 | (1.32-1.55) |
| rs2074840 | 2 | 242141719 | C | T | 0.306 | 1.054 | (1.04-1.07) |
| rs76832527 | 2 | 242157241 | A | G | 0.171 | 1.115 | (1.09-1.14) |
| rs6550597 | 3 | 18738940 | A | G | 0.713 | 1.033 | (1.02-1.05) |
| rs7618603 | 3 | 23153062 | A | C | 0.169 | 1.057 | (1.04-1.08) |
| rs34680713 | 3 | 49621718 | A | AT | 0.182 | 1.062 | (1.04-1.08) |
| rs13091518 | 3 | 70796696 | T | C | 0.584 | 1.043 | (1.03-1.06) |
| rs143745027 | 3 | 87144017 | G | A | 0.064 | 1.165 | (1.13-1.20) |
| rs7628934 | 3 | 87175984 | C | T | 0.501 | 1.097 | (1.08-1.11) |
| rs114810266 | 3 | 87399362 | A | G | 0.977 | 1.168 | (1.11-1.23) |
| rs1283104 | 3 | 106962521 | G | C | 0.377 | 1.05 | (1.03-1.07) |
| rs151038334 | 3 | 107193337 | C | T | 0.918 | 1.075 | (1.05-1.11) |
| rs2271494 | 3 | 113300183 | A | T | 0.566 | 1.088 | (1.07-1.10) |
| rs2811476 | 3 | 127898501 | C | A | 0.266 | 1.105 | (1.09-1.12) |
| rs35006112 | 3 | 128213994 | G | A | 0.840 | 1.068 | (1.05-1.09) |
| rs1457063 | 3 | 137562823 | A | G | 0.616 | 1.039 | (1.02-1.06) |
| rs7650602 | 3 | 141147414 | C | T | 0.446 | 1.048 | (1.03-1.06) |
| rs145053401 | 3 | 152011745 | C | CAT | 0.895 | 1.086 | (1.06-1.11) |
| rs2293607 | 3 | 169482335 | T | C | 0.749 | 1.059 | (1.04-1.08) |
| rs78416326 | 3 | 170074517 | G | C | 0.793 | 1.21 | (1.19-1.23) |
| rs577952184 | 3 | 170083540 | C | CTTTTT | 0.882 | 1.102 | (1.08-1.13) |
| rs11717887 | 3 | 172381777 | C | T | 0.366 | 1.044 | (1.03-1.06) |
| rs17804499 | 4 | 74442349 | G | C | 0.947 | 1.227 | (1.18-1.27) |
| rs13142786 | 4 | 74477135 | T | A | 0.475 | 1.081 | (1.06-1.10) |
| rs6853490 | 4 | 95544718 | G | A | 0.442 | 1.082 | (1.07-1.10) |
| rs7679673 | 4 | 106061534 | C | A | 0.592 | 1.105 | (1.09-1.12) |
| rs17035310 | 4 | 106064754 | C | T | 0.869 | 1.069 | (1.04-1.10) |
| rs77821238 | 4 | 140948835 | C | T | 0.835 | 1.054 | (1.03-1.08) |
| rs72725734 | 4 | 146879237 | G | A | 0.158 | 1.04 | (1.02-1.06) |
| rs147762399 | 4 | 152030340 | T | C | 0.035 | 1.099 | (1.06-1.14) |
| rs2242652 | 5 | 1280028 | G | A | 0.793 | 1.141 | (1.12-1.17) |
| rs71595003 | 5 | 1292118 | A | G | 0.031 | 1.188 | (1.14-1.24) |
| rs2736098 | 5 | 1294086 | T | C | 0.260 | 1.075 | (1.06-1.09) |
| rs4975758 | 5 | 1891174 | G | C | 0.475 | 1.082 | (1.06-1.10) |
| rs71599622 | 5 | 14372362 | T | TGA | 0.302 | 1.058 | (1.04-1.08) |
| rs10941370 | 5 | 37833419 | T | C | 0.452 | 1.034 | (1.02-1.05) |
| rs1482675 | 5 | 44368506 | T | C | 0.311 | 1.041 | (1.02-1.06) |
| rs9292122 | 5 | 56087910 | A | G | 0.712 | 1.042 | (1.02-1.06) |
| rs10793821 | 5 | 133836209 | T | C | 0.578 | 1.059 | (1.04-1.07) |
| rs76551843 | 5 | 169172133 | A | G | 0.991 | 1.311 | (1.19-1.44) |
| rs9686557 | 5 | 172959030 | C | A | 0.444 | 1.045 | (1.03-1.06) |
| rs61739424 | 5 | 177683905 | G | A | 0.958 | 1.101 | (1.06-1.15) |
| rs2672843 | 5 | 177891551 | G | A | 0.411 | 1.047 | (1.03-1.06) |
| rs2814811 | 6 | 1670985 | A | G | 0.403 | 1.043 | (1.03-1.06) |
| rs2018336 | 6 | 11217897 | T | C | 0.780 | 1.066 | (1.05-1.08) |
| rs6927369 | 6 | 21330689 | C | T | 0.803 | 1.053 | (1.03-1.07) |
| rs4269363 | 6 | 21471490 | G | A | 0.568 | 1.036 | (1.02-1.05) |
| rs12665509 | 6 | 21878849 | A | C | 0.453 | 1.043 | (1.03-1.06) |
| rs35159226 | 6 | 26649830 | C | CT | 0.259 | 1.043 | (1.03-1.06) |
| rs62407547 | 6 | 30216712 | C | T | 0.256 | 1.06 | (1.01-1.12) |
| rs9275160 | 6 | 32652620 | A | G | 0.337 | 1.039 | (1.02-1.06) |
| rs9469899 | 6 | 34793124 | A | G | 0.358 | 1.048 | (1.03-1.06) |
| rs4714485 | 6 | 41536587 | G | T | 0.276 | 1.087 | (1.07-1.10) |
| rs9472120 | 6 | 43709785 | C | T | 0.491 | 1.042 | (1.03-1.06) |
| rs9443189 | 6 | 76495882 | A | G | 0.858 | 1.065 | (1.04-1.09) |
| rs2038542 | 6 | 109295293 | C | T | 0.146 | 1.077 | (1.06-1.10) |
| rs339351 | 6 | 117200434 | C | A | 0.694 | 1.093 | (1.08-1.11) |
| rs150184171 | 6 | 134292717 | G | GTGTTGT | 0.531 | 1.049 | (1.03-1.07) |
| rs13215045 | 6 | 153447516 | C | T | 0.684 | 1.076 | (1.06-1.09) |
| rs963800 | 6 | 160150279 | C | T | 0.781 | 1.062 | (1.04-1.08) |
| rs4646284 | 6 | 160581543 | TG | T | 0.294 | 1.217 | (1.20-1.24) |
| rs4513875 | 7 | 1928159 | T | C | 0.382 | 1.041 | (1.03-1.06) |
| rs11452686 | 7 | 20414110 | T | TA | 0.561 | 1.048 | (1.03-1.06) |
| rs9655205 | 7 | 20999211 | C | A | 0.222 | 1.096 | (1.08-1.12) |
| rs35389879 | 7 | 21812043 | T | G | 0.415 | 1.041 | (1.03-1.06) |
| rs6956484 | 7 | 27564862 | A | C | 0.615 | 1.056 | (1.04-1.07) |
| rs10486567 | 7 | 27976563 | G | A | 0.763 | 1.121 | (1.10-1.14) |
| rs12701838 | 7 | 40877473 | A | G | 0.730 | 1.07 | (1.05-1.09) |
| rs834608 | 7 | 47451918 | A | T | 0.576 | 1.053 | (1.04-1.07) |
| rs6955627 | 7 | 92577760 | C | T | 0.914 | 1.054 | (1.03-1.08) |
| rs4727386 | 7 | 97688440 | A | G | 0.460 | 1.105 | (1.09-1.12) |
| rs870167 | 8 | 8498803 | G | A | 0.072 | 1.062 | (1.03-1.09) |
| rs2572375 | 8 | 11217455 | C | T | 0.704 | 1.046 | (1.03-1.06) |
| rs6557704 | 8 | 23470785 | A | G | 0.278 | 1.072 | (1.05-1.09) |
| rs1160267 | 8 | 23529521 | G | A | 0.429 | 1.153 | (1.14-1.17) |
| rs141811748 | 8 | 25894201 | C | CCAAA | 0.143 | 1.081 | (1.06-1.10) |
| rs12677206 | 8 | 26063165 | A | C | 0.333 | 1.037 | (1.02-1.05) |
| rs11467 | 8 | 38644914 | A | G | 0.628 | 1.041 | (1.03-1.06) |
| rs73700335 | 8 | 108923107 | G | A | 0.837 | 1.053 | (1.03-1.07) |
| rs57588856 | 8 | 127836925 | C | CAA | 0.641 | 1.055 | (1.04-1.07) |
| rs6984837 | 8 | 127901649 | G | A | 0.319 | 1.079 | (1.06-1.10) |
| rs7011138 | 8 | 127922200 | A | T | 0.821 | 0.956 | (0.93-0.99) |
| rs7463326 | 8 | 128027954 | G | A | 0.745 | 1.167 | (1.14-1.19) |
| rs72725854 | 8 | 128074815 | T | A | 0.000 | NA | (NA-NA) |
| rs77541621 | 8 | 128077146 | A | G | 0.024 | 1.893 | (1.80-1.99) |
| rs72725879 | 8 | 128103969 | T | C | 0.185 | 1.229 | (1.19-1.26) |
| rs183373024 | 8 | 128104117 | G | A | 0.007 | 2.269 | (2.09-2.47) |
| rs78809737 | 8 | 128104218 | G | A | 0.953 | 1.083 | (1.05-1.11) |
| rs201057014 | 8 | 128325355 | T | C | 0.127 | 1.068 | (1.04-1.10) |
| rs17464492 | 8 | 128342866 | A | G | 0.717 | 1.119 | (1.10-1.14) |
| rs6983267 | 8 | 128413305 | G | T | 0.511 | 1.175 | (1.16-1.19) |
| rs10090154 | 8 | 128532137 | T | C | 0.104 | 1.337 | (1.23-1.45) |
| rs34265760 | 8 | 128535543 | T | C | 0.979 | 1.08 | (1.03-1.13) |
| rs12549761 | 8 | 128540776 | C | G | 0.876 | 1.2 | (1.17-1.23) |
| rs34540271 | 9 | 18554773 | C | T | 0.698 | 1.051 | (1.03-1.07) |
| rs10122990 | 9 | 19072246 | C | A | 0.373 | 1.059 | (1.04-1.08) |
| rs17694493 | 9 | 22041998 | G | C | 0.136 | 1.081 | (1.06-1.10) |
| rs10122495 | 9 | 34049779 | T | A | 0.294 | 1.055 | (1.04-1.07) |
| rs142727307 | 9 | 82090723 | T | TG | 0.109 | 1.072 | (1.05-1.10) |
| rs4451364 | 9 | 109532734 | A | G | 0.767 | 1.05 | (1.03-1.07) |
| rs817872 | 9 | 110144887 | C | T | 0.275 | 1.067 | (1.05-1.08) |
| rs143655302 | 9 | 110290217 | G | A | 0.979 | 1.304 | (1.23-1.38) |
| rs2241167 | 9 | 130430116 | A | G | 0.510 | 1.04 | (1.03-1.06) |
| rs12634 | 9 | 132573536 | T | G | 0.236 | 1.056 | (1.04-1.07) |
| rs12781100 | 10 | 838636 | T | C | 0.159 | 1.076 | (1.05-1.10) |
| rs7075427 | 10 | 46104943 | A | C | 0.918 | 1.125 | (1.09-1.16) |
| rs11599847 | 10 | 47599029 | T | C | 0.960 | 1.122 | (1.08-1.17) |
| rs10993994 | 10 | 51549496 | T | C | 0.381 | 1.228 | (1.21-1.25) |
| rs11817544 | 10 | 80236999 | C | A | 0.942 | 1.071 | (1.04-1.11) |
| rs12412705 | 10 | 80835998 | C | T | 0.072 | 1.039 | (1.01-1.07) |
| rs1935581 | 10 | 90195149 | C | T | 0.629 | 1.046 | (1.03-1.06) |
| rs12262998 | 10 | 104428716 | C | T | 0.676 | 1.071 | (1.05-1.09) |
| rs10885396 | 10 | 114711755 | T | C | 0.538 | 1.045 | (1.03-1.06) |
| rs4558107 | 10 | 122794926 | A | G | 0.391 | 1.061 | (1.04-1.08) |
| rs140783917 | 10 | 122834482 | C | T | 0.999 | 1.098 | (0.89-1.36) |
| rs10788167 | 10 | 123054018 | T | A | 0.760 | 1.063 | (1.04-1.08) |
| rs10749415 | 10 | 123185303 | A | G | 0.945 | 1.104 | (1.07-1.14) |
| rs12769682 | 10 | 126697494 | C | G | 0.266 | 1.057 | (1.04-1.07) |
| rs1881502 | 11 | 1507512 | T | C | 0.190 | 1.058 | (1.04-1.08) |
| rs11043143 | 11 | 2234093 | T | C | 0.198 | 1.182 | (1.16-1.20) |
| rs61890184 | 11 | 7547587 | A | G | 0.123 | 1.075 | (1.05-1.10) |
| rs68010938 | 11 | 47428209 | T | TA | 0.297 | 1.045 | (1.03-1.06) |
| rs1048374 | 11 | 58902679 | G | A | 0.008 | 1.036 | (0.92-1.17) |
| rs2277283 | 11 | 61908440 | C | T | 0.315 | 1.055 | (1.04-1.07) |
| rs12785905 | 11 | 66951965 | C | G | 0.047 | 1.123 | (1.08-1.16) |
| rs3018690 | 11 | 68882926 | T | C | 0.448 | 1.046 | (1.03-1.06) |
| rs11825796 | 11 | 68980788 | A | G | 0.261 | 1.099 | (1.08-1.12) |
| rs11228580 | 11 | 69002342 | C | T | 0.158 | 1.267 | (1.24-1.29) |
| rs3918298 | 11 | 69463273 | A | G | 0.025 | 1.176 | (1.12-1.23) |
| rs56159348 | 11 | 76267331 | T | G | 0.674 | 1.059 | (1.04-1.08) |
| rs11568818 | 11 | 102401661 | T | C | 0.550 | 1.074 | (1.06-1.09) |
| rs74911261 | 11 | 108357137 | A | G | 0.023 | 1.153 | (1.10-1.21) |
| rs5794883 | 11 | 113700546 | C | CA | 0.699 | 1.072 | (1.05-1.09) |
| rs138466039 | 11 | 125054793 | T | C | 0.009 | 1.324 | (1.22-1.44) |
| rs878987 | 11 | 134266372 | G | A | 0.147 | 1.074 | (1.05-1.10) |
| rs2066827 | 12 | 12871099 | T | G | 0.756 | 1.059 | (1.04-1.08) |
| rs77216612 | 12 | 12877983 | A | G | 0.728 | 1.028 | (1.01-1.05) |
| rs10845938 | 12 | 14416918 | G | A | 0.557 | 1.061 | (1.05-1.08) |
| rs80130819 | 12 | 48419618 | A | C | 0.909 | 1.095 | (1.07-1.12) |
| rs56222401 | 12 | 49672714 | G | A | 0.255 | 1.076 | (1.06-1.09) |
| rs113925811 | 12 | 53308932 | A | C | 0.128 | 1.181 | (1.16-1.21) |
| rs187809440 | 12 | 53329231 | T | C | 0.001 | 1.169 | (0.87-1.57) |
| rs7968403 | 12 | 65012824 | T | C | 0.642 | 1.062 | (1.05-1.08) |
| rs4842687 | 12 | 90156377 | A | G | 0.705 | 1.059 | (1.04-1.08) |
| rs77121786 | 12 | 102446675 | G | T | 0.200 | 1.046 | (1.03-1.07) |
| rs1270884 | 12 | 114685571 | A | G | 0.483 | 1.071 | (1.06-1.09) |
| rs7295014 | 12 | 133067989 | G | A | 0.341 | 1.051 | (1.03-1.07) |
| rs1327653 | 13 | 51076440 | T | C | 0.258 | 1.039 | (1.02-1.06) |
| rs7489409 | 13 | 73716861 | C | T | 0.186 | 1.071 | (1.05-1.09) |
| rs7336001 | 13 | 73995877 | G | C | 0.911 | 1.108 | (1.08-1.14) |
| rs75823044 | 13 | 110360784 | T | C | 0.000 | 1.229 | (0.42-3.58) |
| rs1004030 | 14 | 23305649 | T | C | 0.584 | 1.048 | (1.03-1.06) |
| rs6571758 | 14 | 37136194 | G | A | 0.616 | 1.057 | (1.04-1.07) |
| rs11849126 | 14 | 38144592 | G | A | 0.694 | 1.043 | (1.03-1.06) |
| rs4901313 | 14 | 53387109 | G | T | 0.813 | 1.087 | (1.07-1.11) |
| rs8005621 | 14 | 61106699 | G | A | 0.087 | 1.058 | (1.03-1.09) |
| rs79133931 | 14 | 64687926 | T | C | 0.002 | 0.772 | (0.62-0.95) |
| rs2093202 | 14 | 68923908 | A | G | 0.613 | 1.042 | (1.03-1.06) |
| rs767127 | 14 | 69134264 | G | A | 0.497 | 1.054 | (1.04-1.07) |
| rs17565772 | 14 | 70756333 | G | A | 0.466 | 1.042 | (1.03-1.06) |
| rs11561564 | 15 | 40965044 | G | A | 0.838 | 1.065 | (1.04-1.09) |
| rs33984059 | 15 | 56385868 | A | G | 0.977 | 1.192 | (1.13-1.26) |
| rs74634457 | 15 | 66835704 | G | A | 0.256 | 1.052 | (1.03-1.07) |
| rs8023793 | 15 | 66942093 | A | C | 0.511 | 1.013 | (1.00-1.03) |
| rs12913603 | 15 | 70668824 | A | C | 0.481 | 1.038 | (1.02-1.05) |
| rs7188897 | 16 | 54469331 | T | C | 0.350 | 1.048 | (1.03-1.06) |
| rs13380763 | 16 | 54678305 | C | T | 0.814 | 1.053 | (1.03-1.07) |
| rs11863709 | 16 | 57654576 | C | T | 0.959 | 1.153 | (1.11-1.20) |
| rs28709974 | 16 | 79847632 | C | T | 0.058 | 1.089 | (1.06-1.12) |
| rs8052913 | 16 | 82166181 | C | T | 0.385 | 1.044 | (1.03-1.06) |
| rs684232 | 17 | 618965 | C | T | 0.352 | 1.086 | (1.07-1.10) |
| rs78378222 | 17 | 7571752 | G | T | 0.012 | 1.262 | (1.18-1.35) |
| rs28441558 | 17 | 7803118 | C | T | 0.055 | 1.168 | (1.13-1.21) |
| rs72811270 | 17 | 12585459 | A | G | 0.120 | 1.056 | (1.03-1.08) |
| rs4795646 | 17 | 30092898 | G | A | 0.773 | 1.064 | (1.04-1.08) |
| rs3110641 | 17 | 36047417 | A | G | 0.221 | 1.057 | (1.04-1.08) |
| rs11649743 | 17 | 36074979 | G | A | 0.805 | 1.102 | (1.08-1.12) |
| rs11263763 | 17 | 36103565 | A | G | 0.529 | 1.219 | (1.20-1.24) |
| rs138213197 | 17 | 46805705 | T | C | 0.002 | 3.976 | (3.38-4.68) |
| rs2960158 | 17 | 47380305 | T | C | 0.766 | 1.039 | (1.02-1.06) |
| rs565189650 | 17 | 47398245 | T | C | 0.073 | 1.098 | (1.07-1.13) |
| rs12938538 | 17 | 56426027 | T | C | 0.558 | 1.043 | (1.03-1.06) |
| rs148511027 | 17 | 69117532 | G | GTTAT | 0.473 | 1.17 | (1.15-1.19) |
| rs8089411 | 18 | 51771322 | C | T | 0.444 | 1.044 | (1.03-1.06) |
| rs35283980 | 18 | 56745999 | G | C | 0.305 | 1.045 | (1.03-1.06) |
| rs533722308 | 18 | 60961193 | CT | C | 0.417 | 1.05 | (1.03-1.07) |
| rs11876000 | 18 | 73035513 | T | G | 0.414 | 1.043 | (1.03-1.06) |
| rs9959454 | 18 | 76770820 | A | G | 0.732 | 1.085 | (1.07-1.10) |
| rs10412482 | 19 | 17228554 | C | T | 0.706 | 1.052 | (1.03-1.07) |
| rs17501397 | 19 | 32168343 | C | T | 0.911 | 1.081 | (1.05-1.11) |
| rs59710626 | 19 | 38548094 | G | T | 0.864 | 1.059 | (1.04-1.08) |
| rs4802297 | 19 | 38738130 | G | C | 0.489 | 1.099 | (1.08-1.12) |
| rs11673591 | 19 | 41985931 | T | A | 0.737 | 1.091 | (1.07-1.11) |
| rs2659051 | 19 | 51345568 | G | C | 0.798 | 1.088 | (1.07-1.11) |
| rs61752561 | 19 | 51361382 | G | A | 0.958 | 1.157 | (1.11-1.20) |
| rs76765083 | 19 | 51362715 | T | G | 0.923 | 1.308 | (1.27-1.35) |
| rs6039055 | 20 | 867324 | A | G | 0.711 | 1.033 | (1.01-1.05) |
| rs11480453 | 20 | 31347512 | C | CA | 0.605 | 1.044 | (1.03-1.06) |
| rs6141551 | 20 | 34006970 | C | T | 0.609 | 1.04 | (1.02-1.06) |
| rs73909841 | 20 | 49548807 | T | C | 0.928 | 1.107 | (1.08-1.14) |
| rs6126986 | 20 | 52464719 | C | T | 0.479 | 1.065 | (1.05-1.08) |
| rs139135938 | 20 | 61001061 | TGGCAGTGGCAGC | T | 0.673 | 1.045 | (1.03-1.06) |
| rs381331 | 20 | 62229989 | A | G | 0.611 | 1.054 | (1.04-1.07) |
| rs3787099 | 20 | 62307517 | A | G | 0.914 | 1.108 | (1.08-1.14) |
| rs1058319 | 20 | 62374389 | C | T | 0.864 | 1.117 | (1.09-1.14) |
| rs11701433 | 21 | 40296411 | C | T | 0.334 | 1.039 | (1.02-1.06) |
| rs61735792 | 21 | 42866332 | A | G | 0.016 | 1.263 | (1.19-1.34) |
| rs9978557 | 21 | 42882462 | C | T | 0.902 | 1.098 | (1.07-1.13) |
| rs1978060 | 22 | 19749525 | G | A | 0.615 | 1.055 | (1.04-1.07) |
| rs9625483 | 22 | 28888939 | A | G | 0.027 | 1.149 | (1.10-1.20) |
| rs17886163 | 22 | 29091147 | C | A | 0.000 | NA | (NA-NA) |
| rs555607708 | 22 | 29091856 | A | AG | 0.004 | 1.477 | (1.29-1.69) |
| rs138708 | 22 | 39138332 | G | A | 0.980 | 1.142 | (1.08-1.21) |
| rs34584683 | 22 | 40499107 | T | A | 0.206 | 1.072 | (1.05-1.09) |
| rs6003062 | 22 | 43499741 | G | A | 0.933 | 1.17 | (1.13-1.21) |
| rs5759167 | 22 | 43500212 | G | T | 0.502 | 1.132 | (1.12-1.15) |
| rs9615099 | 22 | 45698149 | T | A | 0.745 | 1.038 | (1.02-1.06) |
| rs960417 | X | 9811095 | A | G | 0.713 | 1.046 | (1.03-1.06) |
| rs17321482 | X | 11482634 | C | T | 0.866 | 1.061 | (1.04-1.08) |
| rs5972255 | X | 30896320 | T | C | 0.233 | 1.031 | (1.02-1.04) |
| rs11338635 | X | 51245276 | GA | G | 0.364 | 1.106 | (1.09-1.12) |
| rs5943724 | X | 52695895 | G | A | 0.629 | 1.056 | (1.04-1.07) |
| rs4826594 | X | 54454406 | A | G | 0.093 | 0.985 | (0.96-1.01) |
| rs5919393 | X | 66825357 | T | C | 0.845 | 1.06 | (1.04-1.08) |
| rs371707439 | X | 70139908 | A | G | 0.257 | 1.057 | (1.04-1.07) |

TABLE 2
African polymorphisms.

| | | | | | Effect | | |
|---|---|---|---|---|---|---|---|
| | | | Effect | Reference | Allele | | |
| Polymorphism | Chromosome | Position | Allele | Allele | Frequency | OR | (95% CI) |
| rs7542260 | 1 | 5743196 | T | C | 0.439 | 0.978 | (0.94-1.02) |
| rs2847344 | 1 | 10564675 | A | G | 0.703 | 1.027 | (0.98-1.07) |
| rs10803412 | 1 | 16376831 | C | T | 0.594 | 1.055 | (1.01-1.10) |
| rs544780844 | 1 | 46251655 | T | C | 0.098 | 1.103 | (1.03-1.19) |
| rs56391074 | 1 | 88210715 | AT | A | 0.722 | 1.066 | (1.02-1.12) |
| rs1811698 | 1 | 150772613 | C | T | 0.448 | 1.043 | (1.00-1.09) |
| rs607518 | 1 | 150954671 | A | G | 0.068 | 1.11 | (1.02-1.21) |
| rs10127983 | 1 | 153923276 | T | C | 0.223 | 1.05 | (1.00-1.10) |
| rs56103503 | 1 | 154980351 | T | C | 0.086 | 1.07 | (0.99-1.16) |
| rs147847496 | 1 | 155118588 | C | T | 0.994 | NA | (NA-NA) |
| rs184104770 | 1 | 155690186 | A | C | 0.004 | NA | (NA-NA) |
| rs80237341 | 1 | 157119915 | C | G | 0.007 | NA | (NA-NA) |
| rs6660538 | 1 | 163295678 | A | C | 0.474 | 1.062 | (1.02-1.10) |
| rs4075646 | 1 | 167135941 | T | A | 0.147 | 1.074 | (1.02-1.14) |
| rs507603 | 1 | 179897070 | A | C | 0.314 | 1.043 | (1.00-1.09) |
| rs34295433 | 1 | 183032447 | CTAAG | C | 0.367 | 1.028 | (0.98-1.07) |
| rs138638958 | 1 | 204030362 | TTTTG | T | 0.691 | 1.034 | (0.99-1.08) |
| rs4245739 | 1 | 204518842 | A | C | 0.761 | 1.04 | (0.99-1.09) |
| rs708723 | 1 | 205739266 | C | T | 0.744 | 1.076 | (1.03-1.13) |
| rs11686272 | 2 | 8598444 | T | G | 0.221 | 1.009 | (0.96-1.06) |
| rs73913932 | 2 | 10094526 | G | A | 0.184 | 1.059 | (1.01-1.11) |
| rs1990613 | 2 | 10781975 | T | C | 0.469 | 1.039 | (1.00-1.08) |
| rs7602028 | 2 | 16016503 | C | A | 0.804 | 1.057 | (1.00-1.11) |
| rs9306894 | 2 | 20878105 | G | A | 0.115 | 1.101 | (1.03-1.17) |
| rs6738169 | 2 | 43064555 | C | G | 0.804 | 1.065 | (1.01-1.12) |
| rs7591218 | 2 | 43637998 | A | G | 0.325 | 1.008 | (0.97-1.05) |
| rs28514770 | 2 | 43851282 | C | G | 0.428 | 1.036 | (0.99-1.08) |
| rs11125927 | 2 | 62752975 | G | A | 0.163 | 1.116 | (1.06-1.18) |
| rs58235267 | 2 | 63277843 | G | C | 0.447 | 1.122 | (1.08-1.17) |
| rs139283528 | 2 | 63938756 | G | A | 0.996 | NA | (NA-NA) |
| rs74702681 | 2 | 66652885 | T | C | 0.004 | NA | (NA-NA) |
| rs2028900 | 2 | 85767735 | C | T | 0.321 | 1.094 | (1.05-1.14) |
| rs2165108 | 2 | 111861993 | A | T | 0.143 | 1.094 | (1.04-1.16) |
| rs11691517 | 2 | 111893096 | T | G | 0.807 | 1.006 | (0.96-1.06) |
| rs111595856 | 2 | 121103598 | T | C | 0.405 | 1.056 | (1.01-1.10) |
| rs10206072 | 2 | 121373466 | G | A | 0.796 | 1.095 | (1.04-1.15) |
| rs16854905 | 2 | 169012955 | C | T | 0.742 | 1.053 | (1.01-1.10) |
| rs77167534 | 2 | 173319930 | C | T | 0.987 | 1.284 | (1.06-1.56) |
| rs34925593 | 2 | 174234547 | C | T | 0.221 | 1.059 | (1.01-1.11) |
| rs1861270 | 2 | 202126615 | G | A | 0.782 | 1.027 | (0.98-1.08) |
| rs12621900 | 2 | 208118301 | C | T | 0.451 | 1.049 | (1.01-1.09) |
| rs74001374 | 2 | 238411293 | C | T | 0.714 | 1.147 | (1.10-1.20) |
| rs2292884 | 2 | 238443226 | G | A | 0.562 | 1.063 | (1.02-1.11) |
| rs77559646 | 2 | 242135265 | A | G | 0.004 | NA | (NA-NA) |
| rs77482050 | 2 | 242139600 | G | A | 0.999 | NA | (NA-NA) |
| rs2074840 | 2 | 242141719 | C | T | 0.485 | 1.019 | (0.98-1.06) |
| rs76832527 | 2 | 242157241 | A | G | 0.043 | 1.143 | (1.03-1.26) |
| rs6550597 | 3 | 18738940 | A | G | 0.726 | 1.071 | (1.02-1.12) |
| rs7618603 | 3 | 23153062 | A | C | 0.461 | 1.047 | (1.01-1.09) |
| rs34680713 | 3 | 49621718 | A | AT | 0.186 | 1.03 | (0.97-1.09) |
| rs13091518 | 3 | 70796696 | T | C | 0.559 | 1.027 | (0.99-1.07) |
| rs143745027 | 3 | 87144017 | G | A | 0.057 | 1.084 | (0.99-1.18) |
| rs7628934 | 3 | 87175984 | C | T | 0.788 | 1.111 | (1.05-1.17) |
| rs114810266 | 3 | 87399362 | A | G | 0.959 | 1.171 | (1.05-1.30) |
| rs1283104 | 3 | 106962521 | G | C | 0.485 | 1.025 | (0.98-1.07) |
| rs151038334 | 3 | 107193337 | C | T | 0.947 | 1.128 | (1.03-1.23) |
| rs2271494 | 3 | 113300183 | A | T | 0.486 | 1.036 | (1.00-1.08) |
| rs2811476 | 3 | 127898501 | C | A | 0.316 | 1.074 | (1.03-1.12) |
| rs35006112 | 3 | 128213994 | G | A | 0.826 | 1.094 | (1.04-1.15) |
| rs1457063 | 3 | 137562823 | A | G | 0.253 | 1.071 | (1.02-1.12) |
| rs7650602 | 3 | 141147414 | C | T | 0.688 | 1.046 | (1.00-1.09) |
| rs145053401 | 3 | 152011745 | C | CAT | 0.912 | 1.115 | (1.04-1.20) |
| rs2293607 | 3 | 169482335 | T | C | 0.929 | 1.158 | (1.07-1.26) |
| rs78416326 | 3 | 170074517 | G | C | 0.958 | 1.136 | (1.02-1.27) |
| rs577952184 | 3 | 170083540 | C | CTTTTT | 0.887 | 1.058 | (0.99-1.13) |
| rs11717887 | 3 | 172381777 | C | T | 0.298 | 1.091 | (1.04-1.14) |
| rs17804499 | 4 | 74442349 | G | C | 0.988 | 1.013 | (0.84-1.23) |
| rs13142786 | 4 | 74477135 | T | A | 0.589 | 1.05 | (1.01-1.09) |
| rs6853490 | 4 | 95544718 | G | A | 0.213 | 1.06 | (1.01-1.11) |
| rs7679673 | 4 | 106061534 | C | A | 0.383 | 1.089 | (1.04-1.14) |
| rs17035310 | 4 | 106064754 | C | T | 0.824 | 1.05 | (0.99-1.11) |
| rs77821238 | 4 | 140948835 | C | T | 0.896 | 1.031 | (0.96-1.11) |
| rs72725734 | 4 | 146879237 | G | A | 0.255 | 1.05 | (1.00-1.10) |
| rs147762399 | 4 | 152030340 | T | C | 0.092 | 1.073 | (1.00-1.15) |
| rs2242652 | 5 | 1280028 | G | A | 0.858 | 1.14 | (1.07-1.21) |
| rs71595003 | 5 | 1292118 | A | G | 0.005 | NA | (NA-NA) |
| rs2736098 | 5 | 1294086 | T | C | 0.116 | 1.019 | (0.96-1.09) |
| rs4975758 | 5 | 1891174 | G | C | 0.318 | 1.121 | (1.07-1.17) |
| rs71599622 | 5 | 14372362 | T | TGA | 0.459 | 0.996 | (0.95-1.04) |
| rs10941370 | 5 | 37833419 | T | C | 0.423 | 1.056 | (1.01-1.10) |
| rs1482675 | 5 | 44368506 | T | C | 0.361 | 1.024 | (0.98-1.07) |
| rs9292122 | 5 | 56087910 | A | G | 0.593 | 1.066 | (1.02-1.11) |
| rs10793821 | 5 | 133836209 | T | C | 0.669 | 1.033 | (0.99-1.08) |
| rs76551843 | 5 | 169172133 | A | G | 0.998 | NA | (NA-NA) |
| rs9686557 | 5 | 172959030 | C | A | 0.858 | 0.971 | (0.92-1.03) |
| rs61739424 | 5 | 177683905 | G | A | 0.980 | 1.17 | (1.03-1.34) |
| rs2672843 | 5 | 177891551 | G | A | 0.193 | 1.035 | (0.99-1.09) |
| rs2814811 | 6 | 1670985 | A | G | 0.613 | 1.043 | (1.00-1.09) |
| rs2018336 | 6 | 11217897 | T | C | 0.938 | 1.093 | (1.00-1.19) |
| rs6927369 | 6 | 21330689 | C | T | 0.770 | 1.078 | (1.03-1.13) |
| rs4269363 | 6 | 21471490 | G | A | 0.204 | 1.081 | (1.03-1.14) |
| rs12665509 | 6 | 21878849 | A | C | 0.487 | 1.053 | (1.01-1.10) |
| rs35159226 | 6 | 26649830 | C | CT | 0.301 | 1.053 | (1.01-1.10) |
| rs62407547 | 6 | 30216712 | C | T | 0.054 | 0.895 | (0.79-1.01) |
| rs9275160 | 6 | 32652620 | A | G | 0.279 | 1.068 | (1.02-1.12) |
| rs9469899 | 6 | 34793124 | A | G | 0.670 | 1.021 | (0.98-1.07) |
| rs4714485 | 6 | 41536587 | G | T | 0.470 | 1.106 | (1.06-1.15) |
| rs9472120 | 6 | 43709785 | C | T | 0.556 | 1.043 | (1.00-1.09) |
| rs9443189 | 6 | 76495882 | A | G | 0.473 | 1.105 | (1.06-1.15) |
| rs2038542 | 6 | 109295293 | C | T | 0.268 | 1.029 | (0.98-1.08) |
| rs339351 | 6 | 117200434 | C | A | 0.741 | 1.218 | (1.16-1.28) |
| rs150184171 | 6 | 134292717 | G | GTGTTGT | 0.466 | 1.054 | (1.01-1.10) |
| rs13215045 | 6 | 153447516 | C | T | 0.539 | 1.069 | (1.03-1.11) |
| rs963800 | 6 | 160150279 | C | T | 0.501 | 1.045 | (1.00-1.09) |
| rs4646284 | 6 | 160581543 | TG | T | 0.365 | 1.13 | (1.08-1.18) |
| rs4513875 | 7 | 1928159 | T | C | 0.125 | 1.069 | (1.00-1.14) |
| rs11452686 | 7 | 20414110 | T | TA | 0.372 | 0.991 | (0.95-1.03) |
| rs9655205 | 7 | 20999211 | C | A | 0.148 | 1.067 | (1.01-1.13) |
| rs35389879 | 7 | 21812043 | T | G | 0.111 | 1.012 | (0.95-1.08) |
| rs6956484 | 7 | 27564862 | A | C | 0.488 | 1.037 | (1.00-1.08) |
| rs10486567 | 7 | 27976563 | G | A | 0.709 | 1.104 | (1.06-1.15) |
| rs12701838 | 7 | 40877473 | A | G | 0.891 | 0.976 | (0.92-1.04) |
| rs834608 | 7 | 47451918 | A | T | 0.625 | 1.043 | (1.00-1.09) |
| rs6955627 | 7 | 92577760 | C | T | 0.560 | 1.047 | (1.01-1.09) |
| rs4727386 | 7 | 97688440 | A | G | 0.833 | 1.013 | (0.95-1.08) |
| rs870167 | 8 | 8498803 | G | A | 0.104 | 1.066 | (1.00-1.14) |
| rs2572375 | 8 | 11217455 | C | T | 0.931 | 1.023 | (0.94-1.11) |
| rs6557704 | 8 | 23470785 | A | G | 0.078 | 1.04 | (0.96-1.12) |
| rs1160267 | 8 | 23529521 | G | A | 0.704 | 1.177 | (1.13-1.23) |
| rs141811748 | 8 | 25894201 | C | CCAAA | 0.099 | 1.054 | (0.99-1.13) |
| rs12677206 | 8 | 26063165 | A | C | 0.317 | 1.045 | (1.00-1.09) |
| rs11467 | 8 | 38644914 | A | G | 0.539 | 1.068 | (1.03-1.11) |
| rs73700335 | 8 | 108923107 | G | A | 0.938 | 1.093 | (1.00-1.19) |
| rs57588856 | 8 | 127836925 | C | CAA | 0.356 | 1.036 | (1.00-1.08) |
| rs6984837 | 8 | 127901649 | G | A | 0.090 | 1.105 | (1.02-1.19) |
| rs7011138 | 8 | 127922200 | A | T | 0.956 | 0.927 | (0.83-1.04) |
| rs7463326 | 8 | 128027954 | G | A | 0.840 | 1.274 | (1.20-1.35) |
| rs72725854 | 8 | 128074815 | T | A | 0.061 | 2.087 | (1.94-2.25) |
| rs77541621 | 8 | 128077146 | A | G | 0.004 | NA | (NA-NA) |
| rs72725879 | 8 | 128103969 | T | C | 0.341 | 1.308 | (1.25-1.37) |
| rs183373024 | 8 | 128104117 | G | A | 0.001 | NA | (NA-NA) |
| rs78809737 | 8 | 128104218 | G | A | 0.991 | 1.648 | (0.69-3.94) |
| rs201057014 | 8 | 128325355 | T | C | 0.107 | 1.012 | (0.95-1.08) |
| rs17464492 | 8 | 128342866 | A | G | 0.781 | 1.09 | (1.04-1.14) |
| rs6983267 | 8 | 128413305 | G | T | 0.886 | 1.135 | (1.05-1.23) |
| rs10090154 | 8 | 128532137 | T | C | 0.168 | 1.151 | (1.04-1.27) |
| rs34265760 | 8 | 128535543 | T | C | 0.967 | 1.057 | (0.93-1.20) |
| rs12549761 | 8 | 128540776 | C | G | 0.952 | 1.249 | (1.12-1.40) |
| rs34540271 | 9 | 18554773 | C | T | 0.869 | 1.07 | (1.01-1.14) |
| rs10122990 | 9 | 19072246 | C | A | 0.668 | 1.015 | (0.97-1.06) |
| rs17694493 | 9 | 22041998 | G | C | 0.116 | 1.038 | (0.98-1.10) |
| rs10122495 | 9 | 34049779 | T | A | 0.248 | 1.041 | (0.99-1.09) |
| rs142727307 | 9 | 82090723 | T | TG | 0.359 | 1.093 | (1.05-1.14) |
| rs4451364 | 9 | 109532734 | A | G | 0.652 | 1.051 | (1.01-1.09) |
| rs817872 | 9 | 110144887 | C | T | 0.475 | 1.089 | (1.05-1.13) |
| rs143655302 | 9 | 110290217 | G | A | 0.996 | NA | (NA-NA) |
| rs2241167 | 9 | 130430116 | A | G | 0.662 | 1.023 | (0.98-1.07) |
| rs12634 | 9 | 132573536 | T | G | 0.229 | 1.067 | (1.02-1.12) |
| rs12781100 | 10 | 838636 | T | C | 0.036 | 1.091 | (0.98-1.22) |
| rs7075427 | 10 | 46104943 | A | C | 0.983 | 0.959 | (0.81-1.13) |
| rs11599847 | 10 | 47599029 | T | C | 0.991 | 1.007 | (0.52-1.97) |
| rs10993994 | 10 | 51549496 | T | C | 0.614 | 1.112 | (1.07-1.16) |
| rs11817544 | 10 | 80236999 | C | A | 0.798 | 1.054 | (1.00-1.11) |
| rs12412705 | 10 | 80835998 | C | T | 0.406 | 1.084 | (1.04-1.13) |
| rs1935581 | 10 | 90195149 | C | T | 0.211 | 1.02 | (0.97-1.07) |
| rs12262998 | 10 | 104428716 | C | T | 0.761 | 1.063 | (1.01-1.11) |
| rs10885396 | 10 | 114711755 | T | C | 0.646 | 1.017 | (0.98-1.06) |
| rs4558107 | 10 | 122794926 | A | G | 0.190 | 1.048 | (1.00-1.10) |
| rs140783917 | 10 | 122834482 | C | T | 0.990 | 1.116 | (0.86-1.45) |
| rs10788167 | 10 | 123054018 | T | A | 0.786 | 1.068 | (1.02-1.12) |
| rs10749415 | 10 | 123185303 | A | G | 0.780 | 1.111 | (1.06-1.17) |
| rs12769682 | 10 | 126697494 | C | G | 0.163 | 1.09 | (1.03-1.15) |
| rs1881502 | 11 | 1507512 | T | C | 0.088 | 1.012 | (0.94-1.09) |
| rs11043143 | 11 | 2234093 | T | C | 0.289 | 1.11 | (1.06-1.16) |
| rs61890184 | 11 | 7547587 | A | G | 0.172 | 1.024 | (0.97-1.08) |
| rs68010938 | 11 | 47428209 | T | TA | 0.059 | 0.977 | (0.89-1.07) |
| rs1048374 | 11 | 58902679 | G | A | 0.081 | 1.139 | (1.06-1.22) |
| rs2277283 | 11 | 61908440 | C | T | 0.165 | 1.054 | (1-1.11) |
| rs12785905 | 11 | 66951965 | C | G | 0.011 | 1.003 | (0.78-1.29) |
| rs3018690 | 11 | 68882926 | T | C | 0.744 | 1.017 | (0.97-1.07) |
| rs11825796 | 11 | 68980788 | A | G | 0.512 | 1.069 | (1.03-1.11) |
| rs11228580 | 11 | 69002342 | C | T | 0.158 | 1.31 | (1.24-1.38) |
| rs3918298 | 11 | 69463273 | A | G | 0.241 | 1.081 | (1.03-1.13) |
| rs56159348 | 11 | 76267331 | T | G | 0.802 | 1.042 | (0.99-1.10) |
| rs11568818 | 11 | 102401661 | T | C | 0.547 | 1.077 | (1.03-1.12) |
| rs74911261 | 11 | 108357137 | A | G | 0.005 | NA | (NA-NA) |
| rs5794883 | 11 | 113700546 | C | CA | 0.594 | 1.065 | (1.02-1.11) |
| rs138466039 | 11 | 125054793 | T | C | 0.003 | NA | (NA-NA) |
| rs878987 | 11 | 134266372 | G | A | 0.193 | 1.011 | (0.96-1.06) |
| rs2066827 | 12 | 12871099 | T | G | 0.290 | 1.05 | (1.00-1.10) |
| rs77216612 | 12 | 12877983 | A | G | 0.928 | 1.032 | (0.95-1.12) |
| rs10845938 | 12 | 14416918 | G | A | 0.429 | 1.055 | (1.01-1.10) |
| rs80130819 | 12 | 48419618 | A | C | 0.982 | 1.12 | (0.95-1.31) |
| rs56222401 | 12 | 49672714 | G | A | 0.536 | 1.09 | (1.05-1.13) |
| rs113925811 | 12 | 53308932 | A | C | 0.153 | 1.174 | (1.11-1.24) |
| rs187809440 | 12 | 53329231 | T | C | 0.001 | NA | (NA-NA) |
| rs7968403 | 12 | 65012824 | T | C | 0.837 | 1.011 | (0.96-1.07) |
| rs4842687 | 12 | 90156377 | A | G | 0.610 | 1.043 | (1.00-1.09) |
| rs77121786 | 12 | 102446675 | G | T | 0.186 | 1.049 | (1.00-1.10) |
| rs1270884 | 12 | 114685571 | A | G | 0.197 | 1.061 | (1.01-1.12) |
| rs7295014 | 12 | 133067989 | G | A | 0.457 | 1.067 | (1.03-1.11) |
| rs1327653 | 13 | 51076440 | T | C | 0.308 | 1.029 | (0.98-1.07) |
| rs7489409 | 13 | 73716861 | C | T | 0.160 | 1.115 | (1.06-1.18) |
| rs7336001 | 13 | 73995877 | G | C | 0.985 | 1.018 | (0.85-1.22) |
| rs75823044 | 13 | 110360784 | T | C | 0.021 | 1.53 | (1.35-1.73) |
| rs1004030 | 14 | 23305649 | T | C | 0.624 | 1.01 | (0.97-1.05) |
| rs6571758 | 14 | 37136194 | G | A | 0.579 | 1.073 | (1.03-1.12) |
| rs11849126 | 14 | 38144592 | G | A | 0.764 | 1.066 | (1.02-1.12) |
| rs4901313 | 14 | 53387109 | G | T | 0.718 | 1.053 | (1.01-1.10) |
| rs8005621 | 14 | 61106699 | G | A | 0.340 | 1.078 | (1.03-1.12) |
| rs79133931 | 14 | 64687926 | T | C | 0.019 | 1.057 | (0.91-1.23) |
| rs2093202 | 14 | 68923908 | A | G | 0.399 | 1.031 | (0.99-1.07) |
| rs767127 | 14 | 69134264 | G | A | 0.487 | 1.039 | (1.00-1.08) |
| rs17565772 | 14 | 70756333 | G | A | 0.219 | 1.012 | (0.96-1.06) |
| rs11561564 | 15 | 40965044 | G | A | 0.908 | 1.101 | (1.02-1.19) |
| rs33984059 | 15 | 56385868 | A | G | 0.996 | NA | (NA-NA) |
| rs74634457 | 15 | 66835704 | G | A | 0.087 | 1.038 | (0.97-1.12) |
| rs8023793 | 15 | 66942093 | A | C | 0.336 | 1.081 | (1.04-1.13) |
| rs12913603 | 15 | 70668824 | A | C | 0.633 | 1.001 | (0.96-1.05) |
| rs7188897 | 16 | 54469331 | T | C | 0.139 | 1.042 | (0.98-1.10) |
| rs13380763 | 16 | 54678305 | C | T | 0.827 | 1.072 | (1.02-1.13) |
| rs11863709 | 16 | 57654576 | C | T | 0.965 | 1.075 | (0.95-1.21) |
| rs28709974 | 16 | 79847632 | C | T | 0.075 | 1.092 | (1.01-1.18) |
| rs8052913 | 16 | 82166181 | C | T | 0.580 | 1.023 | (0.98-1.06) |
| rs684232 | 17 | 618965 | C | T | 0.648 | 1.07 | (1.03-1.12) |
| rs78378222 | 17 | 7571752 | G | T | 0.003 | NA | (NA-NA) |
| rs28441558 | 17 | 7803118 | C | T | 0.038 | 1.124 | (1.02-1.24) |
| rs72811270 | 17 | 12585459 | A | G | 0.205 | 1.049 | (1.00-1.10) |
| rs4795646 | 17 | 30092898 | G | A | 0.931 | 0.995 | (0.92-1.08) |
| rs3110641 | 17 | 36047417 | A | G | 0.545 | 1.026 | (0.99-1.07) |
| rs11649743 | 17 | 36074979 | G | A | 0.923 | 1.055 | (0.98-1.14) |
| rs11263763 | 17 | 36103565 | A | G | 0.612 | 1.109 | (1.06-1.16) |
| rs138213197 | 17 | 46805705 | T | C | 0.000 | NA | (NA-NA) |
| rs2960158 | 17 | 47380305 | T | C | 0.837 | 1.041 | (0.99-1.10) |
| rs565189650 | 17 | 47398245 | T | C | 0.087 | 1.174 | (1.09-1.26) |
| rs12938538 | 17 | 56426027 | T | C | 0.623 | 1.044 | (1.00-1.09) |
| rs148511027 | 17 | 69117532 | G | GTTAT | 0.206 | 1.098 | (1.04-1.15) |
| rs8089411 | 18 | 51771322 | C | T | 0.274 | 1.035 | (0.99-1.08) |
| rs35283980 | 18 | 56745999 | G | C | 0.321 | 1.008 | (0.96-1.05) |
| rs533722308 | 18 | 60961193 | CT | C | 0.386 | 1.064 | (1.01-1.12) |
| rs11876000 | 18 | 73035513 | T | G | 0.377 | 1.026 | (0.99-1.07) |
| rs9959454 | 18 | 76770820 | A | G | 0.766 | 1.018 | (0.97-1.07) |
| rs10412482 | 19 | 17228554 | C | T | 0.455 | 1.07 | (1.03-1.12) |
| rs17501397 | 19 | 32168343 | C | T | 0.981 | 0.996 | (0.85-1.17) |
| rs59710626 | 19 | 38548094 | G | T | 0.925 | 1.007 | (0.93-1.09) |
| rs4802297 | 19 | 38738130 | G | C | 0.669 | 1.069 | (1.02-1.12) |
| rs11673591 | 19 | 41985931 | T | A | 0.208 | 1.059 | (1.01-1.11) |
| rs2659051 | 19 | 51345568 | G | C | 0.839 | 1.083 | (1.02-1.14) |
| rs61752561 | 19 | 51361382 | G | A | 0.993 | 0.745 | (0.41-1.35) |
| rs76765083 | 19 | 51362715 | T | G | 0.986 | 1.198 | (1.00-1.44) |
| rs6039055 | 20 | 867324 | A | G | 0.476 | 1.062 | (1.02-1.11) |
| rs11480453 | 20 | 31347512 | C | CA | 0.229 | 1.028 | (0.97-1.09) |
| rs6141551 | 20 | 34006970 | C | T | 0.333 | 1.042 | (1.00-1.09) |
| rs73909841 | 20 | 49548807 | T | C | 0.871 | 1.073 | (1.01-1.14) |
| rs6126986 | 20 | 52464719 | C | T | 0.753 | 1.038 | (0.99-1.09) |
| rs139135938 | 20 | 61001061 | TGGCAGTGGCAGC | T | 0.494 | 1.034 | (0.99-1.08) |
| rs381331 | 20 | 62229989 | A | G | 0.333 | 1.025 | (0.98-1.07) |
| rs3787099 | 20 | 62307517 | A | G | 0.937 | 1.077 | (0.99-1.17) |
| rs1058319 | 20 | 62374389 | C | T | 0.955 | 1.161 | (1.05-1.28) |
| rs11701433 | 21 | 40296411 | C | T | 0.271 | 1.047 | (1.00-1.10) |
| rs61735792 | 21 | 42866332 | A | G | 0.003 | NA | (NA-NA) |
| rs9978557 | 21 | 42882462 | C | T | 0.948 | 1.022 | (0.94-1.12) |
| rs1978060 | 22 | 19749525 | G | A | 0.758 | 1.016 | (0.97-1.06) |
| rs9625483 | 22 | 28888939 | A | G | 0.004 | NA | (NA-NA) |
| rs17886163 | 22 | 29091147 | C | A | 0.015 | 1.594 | (1.37-1.85) |
| rs555607708 | 22 | 29091856 | A | AG | 0.001 | NA | (NA-NA) |
| rs138708 | 22 | 39138332 | G | A | 0.993 | 1.41 | (0.65-3.07) |
| rs34584683 | 22 | 40499107 | T | A | 0.594 | 1.061 | (1.02-1.11) |
| rs6003062 | 22 | 43499741 | G | A | 0.945 | 1.138 | (1.03-1.26) |
| rs5759167 | 22 | 43500212 | G | T | 0.745 | 1.112 | (1.06-1.17) |
| rs9615099 | 22 | 45698149 | T | A | 0.395 | 1.049 | (1.00-1.10) |
| rs960417 | X | 9811095 | A | G | 0.782 | 1.107 | (1.05-1.16) |
| rs17321482 | X | 11482634 | C | T | 0.966 | 1.045 | (0.96-1.14) |
| rs5972255 | X | 30896320 | T | C | 0.481 | 1.028 | (0.99-1.07) |
| rs11338635 | X | 51245276 | GA | G | 0.349 | 1.087 | (1.06-1.12) |
| rs5943724 | X | 52695895 | G | A | 0.778 | 1.003 | (0.97-1.04) |
| rs4826594 | X | 54454406 | A | G | 0.449 | 0.997 | (0.95-1.05) |
| rs5919393 | X | 66825357 | T | C | 0.143 | 1.079 | (1.02-1.14) |
| rs371707439 | X | 70139908 | A | G | 0.118 | 1.022 | (0.97-1.08) |

TABLE 3
East Asian polymorphisms.

| | | | | | Effect | | |
|---|---|---|---|---|---|---|---|
| | | | Effect | Reference | Allele | | |
| Polymorphism | Chromosome | Position | Allele | Allele | Frequency | OR | (95% CI) |
| rs7542260 | 1 | 5743196 | T | C | 0.113 | 1.148 | (1.08-1.22) |
| rs2847344 | 1 | 10564675 | A | G | 0.690 | 1.033 | (0.99-1.08) |
| rs10803412 | 1 | 16376831 | C | T | 0.021 | 1.128 | (0.98-1.30) |
| rs544780844 | 1 | 46251655 | T | C | 0.015 | 1.057 | (0.72-1.55) |
| rs56391074 | 1 | 88210715 | AT | A | 0.751 | 0.993 | (0.90-1.09) |
| rs1811698 | 1 | 150772613 | C | T | 0.810 | 1.091 | (1.04-1.15) |
| rs607518 | 1 | 150954671 | A | G | 0.042 | 0.956 | (0.88-1.04) |
| rs10127983 | 1 | 153923276 | T | C | 0.290 | 1.072 | (1.03-1.12) |
| rs56103503 | 1 | 154980351 | T | C | 0.885 | 1.058 | (1.00-1.12) |
| rs147847496 | 1 | 155118588 | C | T | 1.000 | NA | (NA-NA) |
| rs184104770 | 1 | 155690186 | A | C | 0.000 | NA | (NA-NA) |
| rs80237341 | 1 | 157119915 | C | G | 0.103 | 1.165 | (1.10-1.23) |
| rs6660538 | 1 | 163295678 | A | C | 0.606 | 1.069 | (1.03-1.11) |
| rs4075646 | 1 | 167135941 | T | A | 0.573 | 1.073 | (1.03-1.11) |
| rs507603 | 1 | 179897070 | A | C | 0.234 | 1.096 | (1.05-1.14) |
| rs34295433 | 1 | 183032447 | CTAAG | C | 0.608 | 1.055 | (0.97-1.15) |
| rs138638958 | 1 | 204030362 | TTTTG | T | 0.610 | 1.082 | (1.00-1.18) |
| rs4245739 | 1 | 204518842 | A | C | 0.958 | 1.140 | (1.04-1.25) |
| rs708723 | 1 | 205739266 | C | T | 0.466 | 1.073 | (1.03-1.11) |
| rs11686272 | 2 | 8598444 | T | G | 0.149 | 1.030 | (0.98-1.08) |
| rs73913932 | 2 | 10094526 | G | A | 0.003 | NA | (NA-NA) |
| rs1990613 | 2 | 10781975 | T | C | 0.391 | 1.051 | (1.01-1.09) |
| rs7602028 | 2 | 16016503 | C | A | 0.398 | 1.103 | (1.06-1.15) |
| rs9306894 | 2 | 20878105 | G | A | 0.515 | 1.134 | (1.09-1.18) |
| rs6738169 | 2 | 43064555 | C | G | 0.430 | 1.026 | (0.99-1.06) |
| rs7591218 | 2 | 43637998 | A | G | 0.661 | 1.129 | (1.08-1.18) |
| rs28514770 | 2 | 43851282 | C | G | 0.876 | 1.118 | (1.04-1.20) |
| rs11125927 | 2 | 62752975 | G | A | 0.246 | 1.152 | (1.11-1.20) |
| rs58235267 | 2 | 63277843 | G | C | 0.691 | 1.102 | (1.06-1.15) |
| rs139283528 | 2 | 63938756 | G | A | 1.000 | NA | (NA-NA) |
| rs74702681 | 2 | 66652885 | T | C | 0.003 | NA | (NA-NA) |
| rs2028900 | 2 | 85767735 | C | T | 0.628 | 1.093 | (1.05-1.14) |
| rs2165108 | 2 | 111861993 | A | T | 0.243 | 1.034 | (0.99-1.08) |
| rs11691517 | 2 | 111893096 | T | G | 0.798 | 1.058 | (1.01-1.11) |
| rs111595856 | 2 | 121103598 | T | C | 0.192 | 1.112 | (1.07-1.15) |
| rs10206072 | 2 | 121373466 | G | A | 0.634 | 1.045 | (1.01-1.08) |
| rs16854905 | 2 | 169012955 | C | T | 0.764 | 1.080 | (1.03-1.13) |
| rs77167534 | 2 | 173319930 | C | T | 0.767 | 1.167 | (1.11-1.22) |
| rs34925593 | 2 | 174234547 | C | T | 0.411 | 1.077 | (1.04-1.12) |
| rs1861270 | 2 | 202126615 | G | A | 0.687 | 1.055 | (1.01-1.10) |
| rs12621900 | 2 | 208118301 | C | T | 0.830 | 1.020 | (0.97-1.07) |
| rs74001374 | 2 | 238411293 | C | T | 0.947 | 1.063 | (0.99-1.14) |
| rs2292884 | 2 | 238443226 | G | A | 0.267 | 1.069 | (1.03-1.11) |
| rs77559646 | 2 | 242135265 | A | G | 0.000 | NA | (NA-NA) |
| rs77482050 | 2 | 242139600 | G | A | 1.000 | NA | (NA-NA) |
| rs2074840 | 2 | 242141719 | C | T | 0.887 | 1.058 | (1.00-1.12) |
| rs76832527 | 2 | 242157241 | A | G | 0.100 | 1.012 | (0.96-1.07) |
| rs6550597 | 3 | 18738940 | A | G | 0.531 | 1.084 | (1.04-1.13) |
| rs7618603 | 3 | 23153062 | A | C | 0.168 | 1.095 | (1.04-1.16) |
| rs34680713 | 3 | 49621718 | A | AT | 0.063 | 1.132 | (0.82-1.57) |
| rs13091518 | 3 | 70796696 | T | C | 0.741 | 1.013 | (0.97-1.06) |
| rs143745027 | 3 | 87144017 | G | A | 0.131 | 1.196 | (1.14-1.26) |
| rs7628934 | 3 | 87175984 | C | T | 0.690 | 1.103 | (1.06-1.15) |
| rs114810266 | 3 | 87399362 | A | G | 0.995 | NA | (NA-NA) |
| rs1283104 | 3 | 106962521 | G | C | 0.418 | 1.040 | (1.00-1.08) |
| rs151038334 | 3 | 107193337 | C | T | 0.998 | NA | (NA-NA) |
| rs2271494 | 3 | 113300183 | A | T | 0.209 | 1.087 | (1.04-1.14) |
| rs2811476 | 3 | 127898501 | C | A | 0.115 | 1.138 | (1.08-1.20) |
| rs35006112 | 3 | 128213994 | G | A | 0.631 | 1.134 | (1.09-1.18) |
| rs1457063 | 3 | 137562823 | A | G | 0.700 | 1.062 | (1.02-1.11) |
| rs7650602 | 3 | 141147414 | C | T | 0.331 | 1.080 | (1.04-1.12) |
| rs145053401 | 3 | 152011745 | C | CAT | 0.996 | NA | (NA-NA) |
| rs2293607 | 3 | 169482335 | T | C | 0.412 | 1.050 | (1.01-1.09) |
| rs78416326 | 3 | 170074517 | G | C | 0.781 | 1.162 | (1.11-1.21) |
| rs577952184 | 3 | 170083540 | C | CTTTTT | 0.946 | 1.096 | (0.92-1.31) |
| rs11717887 | 3 | 172381777 | C | T | 0.336 | 1.010 | (0.97-1.05) |
| rs17804499 | 4 | 74442349 | G | C | 0.999 | NA | (NA-NA) |
| rs13142786 | 4 | 74477135 | T | A | 0.338 | 1.023 | (0.99-1.06) |
| rs6853490 | 4 | 95544718 | G | A | 0.459 | 1.061 | (1.02-1.10) |
| rs7679673 | 4 | 106061534 | C | A | 0.199 | 1.070 | (1.02-1.12) |
| rs17035310 | 4 | 106064754 | C | T | 0.977 | 1.181 | (1.05-1.32) |
| rs77821238 | 4 | 140948835 | C | T | 0.929 | 1.113 | (1.03-1.21) |
| rs72725734 | 4 | 146879237 | G | A | 0.319 | 1.091 | (1.05-1.14) |
| rs147762399 | 4 | 152030340 | T | C | 0.153 | 1.074 | (1.03-1.12) |
| rs2242652 | 5 | 1280028 | G | A | 0.810 | 1.164 | (1.12-1.21) |
| rs71595003 | 5 | 1292118 | A | G | 0.000 | NA | (NA-NA) |
| rs2736098 | 5 | 1294086 | T | C | 0.327 | 1.079 | (1.03-1.13) |
| rs4975758 | 5 | 1891174 | G | C | 0.400 | 1.226 | (1.18-1.27) |
| rs71599622 | 5 | 14372362 | T | TGA | 0.428 | 1.037 | (0.94-1.14) |
| rs10941370 | 5 | 37833419 | T | C | 0.615 | 1.041 | (1.00-1.08) |
| rs1482675 | 5 | 44368506 | T | C | 0.315 | 1.022 | (0.98-1.06) |
| rs9292122 | 5 | 56087910 | A | G | 0.255 | 1.005 | (0.96-1.06) |
| rs10793821 | 5 | 133836209 | T | C | 0.731 | 1.044 | (1.00-1.09) |
| rs76551843 | 5 | 169172133 | A | G | 1.000 | NA | (NA-NA) |
| rs9686557 | 5 | 172959030 | C | A | 0.317 | 0.992 | (0.95-1.03) |
| rs61739424 | 5 | 177683905 | G | A | 0.904 | 1.095 | (1.02-1.17) |
| rs2672843 | 5 | 177891551 | G | A | 0.082 | 1.084 | (1.02-1.15) |
| rs2814811 | 6 | 1670985 | A | G | 0.288 | 1.040 | (1.00-1.08) |
| rs2018336 | 6 | 11217897 | T | C | 0.934 | 0.990 | (0.91-1.07) |
| rs6927369 | 6 | 21330689 | C | T | 0.348 | 1.096 | (1.06-1.14) |
| rs4269363 | 6 | 21471490 | G | A | 0.292 | 1.082 | (1.04-1.12) |
| rs12665509 | 6 | 21878849 | A | C | 0.309 | 1.028 | (0.99-1.07) |
| rs35159226 | 6 | 26649830 | C | CT | 0.290 | 1.016 | (0.92-1.12) |
| rs62407547 | 6 | 30216712 | C | T | 0.185 | 1.113 | (0.91-1.37) |
| rs9275160 | 6 | 32652620 | A | G | 0.426 | 1.054 | (0.98-1.14) |
| rs9469899 | 6 | 34793124 | A | G | 0.388 | 1.055 | (1.01-1.10) |
| rs4714485 | 6 | 41536587 | G | T | 0.378 | 1.146 | (1.10-1.19) |
| rs9472120 | 6 | 43709785 | C | T | 0.605 | 1.014 | (0.98-1.05) |
| rs9443189 | 6 | 76495882 | A | G | 0.636 | 1.087 | (1.04-1.13) |
| rs2038542 | 6 | 109295293 | C | T | 0.070 | 1.049 | (0.98-1.13) |
| rs339351 | 6 | 117200434 | C | A | 0.640 | 1.221 | (1.17-1.27) |
| rs150184171 | 6 | 134292717 | G | GTGTTGT | 0.251 | 1.003 | (0.91-1.11) |
| rs13215045 | 6 | 153447516 | C | T | 0.373 | 1.070 | (1.03-1.11) |
| rs963800 | 6 | 160150279 | C | T | 0.599 | 1.087 | (1.05-1.13) |
| rs4646284 | 6 | 160581543 | TG | T | 0.280 | 1.127 | (1.04-1.23) |
| rs4513875 | 7 | 1928159 | T | C | 0.407 | 1.026 | (0.99-1.07) |
| rs11452686 | 7 | 20414110 | T | TA | 0.165 | 1.057 | (0.96-1.17) |
| rs9655205 | 7 | 20999211 | C | A | 0.218 | 1.071 | (1.03-1.12) |
| rs35389879 | 7 | 21812043 | T | G | 0.026 | 1.029 | (0.93-1.14) |
| rs6956484 | 7 | 27564862 | A | C | 0.064 | 1.143 | (1.06-1.23) |
| rs10486567 | 7 | 27976563 | G | A | 0.126 | 1.098 | (1.04-1.16) |
| rs12701838 | 7 | 40877473 | A | G | 0.959 | 1.207 | (1.07-1.36) |
| rs834608 | 7 | 47451918 | A | T | 0.674 | 1.044 | (1.00-1.09) |
| rs6955627 | 7 | 92577760 | C | T | 0.723 | 1.074 | (1.03-1.12) |
| rs4727386 | 7 | 97688440 | A | G | 0.847 | 1.088 | (1.02-1.16) |
| rs870167 | 8 | 8498803 | G | A | 0.312 | 1.068 | (1.02-1.11) |
| rs2572375 | 8 | 11217455 | C | T | 0.994 | 1.762 | (0.70-4.46) |
| rs6557704 | 8 | 23470785 | A | G | 0.083 | 1.112 | (1.04-1.19) |
| rs1160267 | 8 | 23529521 | G | A | 0.337 | 1.270 | (1.23-1.32) |
| rs141811748 | 8 | 25894201 | C | CCAAA | 0.028 | 1.129 | (0.91-1.41) |
| rs12677206 | 8 | 26063165 | A | C | 0.243 | 1.096 | (1.06-1.14) |
| rs11467 | 8 | 38644914 | A | G | 0.929 | 0.948 | (0.86-1.04) |
| rs73700335 | 8 | 108923107 | G | A | 0.972 | 1.099 | (0.96-1.25) |
| rs57588856 | 8 | 127836925 | C | CAA | 0.683 | 0.893 | (0.84-0.95) |
| rs6984837 | 8 | 127901649 | G | A | 0.198 | 1.137 | (1.08-1.19) |
| rs7011138 | 8 | 127922200 | A | T | 0.847 | 0.822 | (0.78-0.87) |
| rs7463326 | 8 | 128027954 | G | A | 0.801 | 1.265 | (1.21-1.32) |
| rs72725854 | 8 | 128074815 | T | A | 0.000 | NA | (NA-NA) |
| rs77541621 | 8 | 128077146 | A | G | 0.001 | NA | (NA-NA) |
| rs72725879 | 8 | 128103969 | T | C | 0.640 | 1.708 | (1.55-1.88) |
| rs183373024 | 8 | 128104117 | G | A | 0.000 | NA | (NA-NA) |
| rs78809737 | 8 | 128104218 | G | A | 1.000 | NA | (NA-NA) |
| rs201057014 | 8 | 128325355 | T | C | 0.240 | 0.923 | (0.83-1.02) |
| rs17464492 | 8 | 128342866 | A | G | 0.943 | 1.176 | (1.07-1.29) |
| rs6983267 | 8 | 128413305 | G | T | 0.366 | 1.148 | (1.11-1.19) |
| rs10090154 | 8 | 128532137 | T | C | 0.154 | 1.612 | (1.32-1.97) |
| rs34265760 | 8 | 128535543 | T | C | 0.906 | 1.215 | (1.14-1.30) |
| rs12549761 | 8 | 128540776 | C | G | 0.790 | 1.098 | (1.05-1.15) |
| rs34540271 | 9 | 18554773 | C | T | 0.855 | 1.027 | (0.98-1.08) |
| rs10122990 | 9 | 19072246 | C | A | 0.157 | 1.071 | (1.02-1.13) |
| rs17694493 | 9 | 22041998 | G | C | 0.024 | 0.924 | (0.79-1.08) |
| rs10122495 | 9 | 34049779 | T | A | 0.263 | 1.044 | (0.99-1.10) |
| rs142727307 | 9 | 82090723 | T | TG | 0.015 | 1.222 | (0.87-1.72) |
| rs4451364 | 9 | 109532734 | A | G | 0.944 | 1.080 | (1.00-1.17) |
| rs817872 | 9 | 110144887 | C | T | 0.304 | 1.070 | (1.03-1.11) |
| rs143655302 | 9 | 110290217 | G | A | 0.999 | NA | (NA-NA) |
| rs2241167 | 9 | 130430116 | A | G | 0.723 | 1.023 | (0.98-1.07) |
| rs12634 | 9 | 132573536 | T | G | 0.252 | 1.075 | (1.04-1.12) |
| rs12781100 | 10 | 838636 | T | C | 0.001 | NA | (NA-NA) |
| rs7075427 | 10 | 46104943 | A | C | 0.997 | NA | (NA-NA) |
| rs11599847 | 10 | 47599029 | T | C | 0.989 | 1.089 | (0.88-1.35) |
| rs10993994 | 10 | 51549496 | T | C | 0.470 | 1.181 | (1.14-1.23) |
| rs11817544 | 10 | 80236999 | C | A | 0.793 | 1.165 | (1.12-1.22) |
| rs12412705 | 10 | 80835998 | C | T | 0.343 | 1.114 | (1.08-1.15) |
| rs1935581 | 10 | 90195149 | C | T | 0.473 | 1.053 | (1.01-1.09) |
| rs12262998 | 10 | 104428716 | C | T | 0.873 | 1.080 | (1.01-1.15) |
| rs10885396 | 10 | 114711755 | T | C | 0.382 | 1.067 | (1.03-1.11) |
| rs4558107 | 10 | 122794926 | A | G | 0.125 | 1.078 | (1.03-1.13) |
| rs140783917 | 10 | 122834482 | C | T | 0.849 | 1.218 | (1.16-1.28) |
| rs10788167 | 10 | 123054018 | T | A | 0.759 | 1.068 | (1.03-1.11) |
| rs10749415 | 10 | 123185303 | A | G | 0.978 | 1.281 | (1.15-1.43) |
| rs12769682 | 10 | 126697494 | C | G | 0.017 | 0.910 | (0.62-1.33) |
| rs1881502 | 11 | 1507512 | T | C | 0.071 | 1.042 | (0.97-1.12) |
| rs11043143 | 11 | 2234093 | T | C | 0.096 | 1.055 | (1.00-1.12) |
| rs61890184 | 11 | 7547587 | A | G | 0.130 | 1.149 | (1.09-1.21) |
| rs68010938 | 11 | 47428209 | T | TA | 0.227 | 1.026 | (0.93-1.13) |
| rs1048374 | 11 | 58902679 | G | A | 0.177 | 1.151 | (1.10-1.21) |
| rs2277283 | 11 | 61908440 | C | T | 0.075 | 1.007 | (0.94-1.08) |
| rs12785905 | 11 | 66951965 | C | G | 0.000 | NA | (NA-NA) |
| rs3018690 | 11 | 68882926 | T | C | 0.425 | 1.074 | (1.04-1.11) |
| rs11825796 | 11 | 68980788 | A | G | 0.017 | 1.144 | (0.97-1.35) |
| rs11228580 | 11 | 69002342 | C | T | 0.005 | 1.173 | (0.78-1.76) |
| rs3918298 | 11 | 69463273 | A | G | 0.017 | 1.161 | (1.07-1.26) |
| rs56159348 | 11 | 76267331 | T | G | 0.849 | 1.083 | (1.03-1.14) |
| rs11568818 | 11 | 102401661 | T | C | 0.912 | 1.060 | (0.98-1.14) |
| rs74911261 | 11 | 108357137 | A | G | 0.001 | NA | (NA-NA) |
| rs5794883 | 11 | 113700546 | C | CA | 0.740 | 0.994 | (0.90-1.10) |
| rs138466039 | 11 | 125054793 | T | C | 0.000 | NA | (NA-NA) |
| rs878987 | 11 | 134266372 | G | A | 0.042 | 1.109 | (1.00-1.23) |
| rs2066827 | 12 | 12871099 | T | G | 0.955 | 1.002 | (0.90-1.11) |
| rs77216612 | 12 | 12877983 | A | G | 0.579 | 1.103 | (1.07-1.14) |
| rs10845938 | 12 | 14416918 | G | A | 0.667 | 1.010 | (0.97-1.05) |
| rs80130819 | 12 | 48419618 | A | C | 0.995 | NA | (NA-NA) |
| rs56222401 | 12 | 49672714 | G | A | 0.707 | 1.094 | (1.04-1.15) |
| rs113925811 | 12 | 53308932 | A | C | 0.005 | NA | (NA-NA) |
| rs187809440 | 12 | 53329231 | T | C | 0.030 | 1.355 | (1.25-1.47) |
| rs7968403 | 12 | 65012824 | T | C | 0.813 | 1.084 | (1.03-1.14) |
| rs4842687 | 12 | 90156377 | A | G | 0.789 | 1.120 | (1.07-1.17) |
| rs77121786 | 12 | 102446675 | G | T | 0.154 | 1.063 | (1.01-1.12) |
| rs1270884 | 12 | 114685571 | A | G | 0.245 | 1.065 | (1.02-1.12) |
| rs7295014 | 12 | 133067989 | G | A | 0.840 | 1.108 | (1.06-1.16) |
| rs1327653 | 13 | 51076440 | T | C | 0.154 | 1.102 | (1.05-1.16) |
| rs7489409 | 13 | 73716861 | C | T | 0.401 | 1.144 | (1.10-1.19) |
| rs7336001 | 13 | 73995877 | G | C | 0.999 | NA | (NA-NA) |
| rs75823044 | 13 | 110360784 | T | C | 0.000 | NA | (NA-NA) |
| rs1004030 | 14 | 23305649 | T | C | 0.648 | 0.995 | (0.96-1.04) |
| rs6571758 | 14 | 37136194 | G | A | 0.741 | 1.097 | (1.05-1.14) |
| rs11849126 | 14 | 38144592 | G | A | 0.854 | 1.057 | (1.00-1.12) |
| rs4901313 | 14 | 53387109 | G | T | 0.997 | NA | (NA-NA) |
| rs8005621 | 14 | 61106699 | G | A | 0.170 | 1.102 | (1.05-1.16) |
| rs79133931 | 14 | 64687926 | T | C | 0.088 | 1.195 | (1.11-1.28) |
| rs2093202 | 14 | 68923908 | A | G | 0.435 | 1.026 | (0.99-1.06) |
| rs767127 | 14 | 69134264 | G | A | 0.815 | 1.068 | (1.02-1.12) |
| rs17565772 | 14 | 70756333 | G | A | 0.280 | 1.077 | (1.03-1.12) |
| rs11561564 | 15 | 40965044 | G | A | 0.318 | 1.059 | (1.02-1.11) |
| rs33984059 | 15 | 56385868 | A | G | 0.999 | NA | (NA-NA) |
| rs74634457 | 15 | 66835704 | G | A | 0.139 | 1.003 | (0.95-1.06) |
| rs8023793 | 15 | 66942093 | A | C | 0.608 | 1.116 | (1.08-1.16) |
| rs12913603 | 15 | 70668824 | A | C | 0.574 | 1.077 | (1.04-1.12) |
| rs7188897 | 16 | 54469331 | T | C | 0.101 | 1.002 | (0.94-1.06) |
| rs13380763 | 16 | 54678305 | C | T | 0.924 | 1.097 | (1.02-1.18) |
| rs11863709 | 16 | 57654576 | C | T | 0.997 | NA | (NA-NA) |
| rs28709974 | 16 | 79847632 | C | T | 0.005 | NA | (NA-NA) |
| rs8052913 | 16 | 82166181 | C | T | 0.617 | 1.046 | (1.01-1.09) |
| rs684232 | 17 | 618965 | C | T | 0.512 | 1.073 | (1.03-1.11) |
| rs78378222 | 17 | 7571752 | G | T | 0.000 | NA | (NA-NA) |
| rs28441558 | 17 | 7803118 | C | T | 0.004 | NA | (NA-NA) |
| rs72811270 | 17 | 12585459 | A | G | 0.086 | 1.093 | (1.02-1.17) |
| rs4795646 | 17 | 30092898 | G | A | 0.306 | 1.056 | (1.01-1.10) |
| rs3110641 | 17 | 36047417 | A | G | 0.290 | 1.079 | (1.04-1.12) |
| rs11649743 | 17 | 36074979 | G | A | 0.693 | 1.150 | (1.11-1.20) |
| rs11263763 | 17 | 36103565 | A | G | 0.713 | 1.310 | (1.26-1.36) |
| rs138213197 | 17 | 46805705 | T | C | 0.000 | NA | (NA-NA) |
| rs2960158 | 17 | 47380305 | T | C | 0.686 | 1.080 | (1.04-1.12) |
| rs565189650 | 17 | 47398245 | T | C | 0.005 | NA | (NA-NA) |
| rs12938538 | 17 | 56426027 | T | C | 0.467 | 1.004 | (0.97-1.04) |
| rs148511027 | 17 | 69117532 | G | GTTAT | 0.320 | 1.090 | (1.00-1.19) |
| rs8089411 | 18 | 51771322 | C | T | 0.745 | 1.071 | (1.02-1.12) |
| rs35283980 | 18 | 56745999 | G | C | 0.502 | 1.035 | (1.00-1.08) |
| rs533722308 | 18 | 60961193 | CT | C | 0.365 | 1.020 | (0.91-1.14) |
| rs11876000 | 18 | 73035513 | T | G | 0.334 | 1.042 | (1.00-1.08) |
| rs9959454 | 18 | 76770820 | A | G | 0.610 | 1.027 | (0.99-1.07) |
| rs10412482 | 19 | 17228554 | C | T | 0.632 | 1.042 | (1.00-1.09) |
| rs17501397 | 19 | 32168343 | C | T | 0.968 | 1.094 | (0.97-1.23) |
| rs59710626 | 19 | 38548094 | G | T | 0.853 | 1.058 | (1.01-1.11) |
| rs4802297 | 19 | 38738130 | G | C | 0.286 | 1.059 | (1.02-1.10) |
| rs11673591 | 19 | 41985931 | T | A | 0.569 | 1.024 | (0.99-1.06) |
| rs2659051 | 19 | 51345568 | G | C | 0.621 | 1.144 | (1.10-1.19) |
| rs61752561 | 19 | 51361382 | G | A | 1.000 | NA | (NA-NA) |
| rs76765083 | 19 | 51362715 | T | G | 0.999 | NA | (NA-NA) |
| rs6039055 | 20 | 867324 | A | G | 0.451 | 1.048 | (1.01-1.09) |
| rs11480453 | 20 | 31347512 | C | CA | 0.129 | 1.117 | (0.97-1.29) |
| rs6141551 | 20 | 34006970 | C | T | 0.747 | 1.033 | (0.99-1.08) |
| rs73909841 | 20 | 49548807 | T | C | 0.925 | 1.202 | (1.11-1.30) |
| rs6126986 | 20 | 52464719 | C | T | 0.420 | 1.021 | (0.98-1.06) |
| rs139135938 | 20 | 61001061 | TGGCAGTGGCAGC | T | 0.575 | 1.088 | (0.99-1.19) |
| rs381331 | 20 | 62229989 | A | G | 0.272 | 1.063 | (1.02-1.10) |
| rs3787099 | 20 | 62307517 | A | G | 0.972 | 1.156 | (1.04-1.29) |
| rs1058319 | 20 | 62374389 | C | T | 0.425 | 1.114 | (1.07-1.15) |
| rs11701433 | 21 | 40296411 | C | T | 0.578 | 1.030 | (0.99-1.08) |
| rs61735792 | 21 | 42866332 | A | G | 0.000 | NA | (NA-NA) |
| rs9978557 | 21 | 42882462 | C | T | 0.998 | NA | (NA-NA) |
| rs1978060 | 22 | 19749525 | G | A | 0.441 | 1.080 | (1.03-1.13) |
| rs9625483 | 22 | 28888939 | A | G | 0.003 | NA | (NA-NA) |
| rs17886163 | 22 | 29091147 | C | A | 0.000 | NA | (NA-NA) |
| rs555607708 | 22 | 29091856 | A | AG | 0.000 | NA | (NA-NA) |
| rs138708 | 22 | 39138332 | G | A | 0.867 | 1.175 | (1.11-1.24) |
| rs34584683 | 22 | 40499107 | T | A | 0.043 | 1.036 | (0.93-1.15) |
| rs6003062 | 22 | 43499741 | G | A | 0.996 | NA | (NA-NA) |
| rs5759167 | 22 | 43500212 | G | T | 0.676 | 1.085 | (1.05-1.13) |
| rs9615099 | 22 | 45698149 | T | A | 0.570 | 1.054 | (1.01-1.10) |
| rs960417 | X | 9811095 | A | G | 0.781 | 1.039 | (1.00-1.08) |
| rs17321482 | X | 11482634 | C | T | 0.997 | NA | (NA-NA) |
| rs5972255 | X | 30896320 | T | C | 0.206 | 1.043 | (1.01-1.08) |
| rs11338635 | X | 51245276 | GA | G | 0.086 | 1.125 | (1.02-1.25) |
| rs5943724 | X | 52695895 | G | A | 0.578 | 1.004 | (0.97-1.04) |
| rs4826594 | X | 54454406 | A | G | 0.445 | 1.060 | (1.03-1.09) |
| rs5919393 | X | 66825357 | T | C | 0.998 | 2.314 | (0.64-8.39) |
| rs371707439 | X | 70139908 | A | G | 0.006 | NA | (NA-NA) |

TABLE 4
Hispanic polymorphisms.

| | | | | | Effect | | |
|---|---|---|---|---|---|---|---|
| | | | Effect | Reference | Allele | | |
| Polymorphism | Chromosome | Position | Allele | Allele | Frequency | OR | (95% CI) |
| rs7542260 | 1 | 5743196 | T | C | 0.157 | 0.994 | (0.89-1.11) |
| rs2847344 | 1 | 10564675 | A | G | 0.540 | 1.032 | (0.96-1.11) |
| rs10803412 | 1 | 16376831 | C | T | 0.126 | 0.954 | (0.85-1.07) |
| rs544780844 | 1 | 46251655 | T | C | 0.100 | 1.155 | (1.01-1.32) |
| rs56391074 | 1 | 88210715 | AT | A | 0.500 | 1.020 | (0.95-1.10) |
| rs1811698 | 1 | 150772613 | C | T | 0.887 | 1.070 | (0.96-1.20) |
| rs607518 | 1 | 150954671 | A | G | 0.116 | 1.011 | (0.9-1.13) |
| rs10127983 | 1 | 153923276 | T | C | 0.399 | 1.023 | (0.95-1.10) |
| rs56103503 | 1 | 154980351 | T | C | 0.490 | 1.061 | (0.99-1.14) |
| rs147847496 | 1 | 155118588 | C | T | 0.984 | 1.030 | (0.77-1.38) |
| rs184104770 | 1 | 155690186 | A | C | 0.006 | NA | (NA-NA) |
| rs80237341 | 1 | 157119915 | C | G | 0.073 | 1.081 | (0.92-1.27) |
| rs6660538 | 1 | 163295678 | A | C | 0.415 | 1.012 | (0.94-1.09) |
| rs4075646 | 1 | 167135941 | T | A | 0.047 | 1.150 | (0.97-1.36) |
| rs507603 | 1 | 179897070 | A | C | 0.123 | 0.968 | (0.86-1.08) |
| rs34295433 | 1 | 183032447 | CTAAG | C | 0.620 | 1.053 | (0.97-1.14) |
| rs138638958 | 1 | 204030362 | TTTTG | T | 0.548 | 1.038 | (0.97-1.12) |
| rs4245739 | 1 | 204518842 | A | C | 0.735 | 1.176 | (1.08-1.28) |
| rs708723 | 1 | 205739266 | C | T | 0.587 | 1.075 | (0.99-1.16) |
| rs11686272 | 2 | 8598444 | T | G | 0.520 | 1.079 | (1.00-1.17) |
| rs73913932 | 2 | 10094526 | G | A | 0.069 | 1.089 | (0.95-1.24) |
| rs1990613 | 2 | 10781975 | T | C | 0.487 | 0.964 | (0.90-1.04) |
| rs7602028 | 2 | 16016503 | C | A | 0.534 | 1.010 | (0.93-1.09) |
| rs9306894 | 2 | 20878105 | G | A | 0.311 | 1.105 | (1.02-1.20) |
| rs6738169 | 2 | 43064555 | C | G | 0.768 | 1.047 | (0.96-1.14) |
| rs7591218 | 2 | 43637998 | A | G | 0.546 | 1.051 | (0.97-1.14) |
| rs28514770 | 2 | 43851282 | C | G | 0.773 | 1.053 | (0.96-1.15) |
| rs11125927 | 2 | 62752975 | G | A | 0.219 | 1.076 | (0.98-1.18) |
| rs58235267 | 2 | 63277843 | G | C | 0.536 | 1.136 | (1.05-1.23) |
| rs139283528 | 2 | 63938756 | G | A | 0.994 | NA | (NA-NA) |
| rs74702681 | 2 | 66652885 | T | C | 0.009 | 0.773 | (0.36-1.68) |
| rs2028900 | 2 | 85767735 | C | T | 0.644 | 1.123 | (1.04-1.22) |
| rs2165108 | 2 | 111861993 | A | T | 0.057 | 1.162 | (1.01-1.34) |
| rs11691517 | 2 | 111893096 | T | G | 0.842 | 1.014 | (0.92-1.12) |
| rs111595856 | 2 | 121103598 | T | C | 0.090 | 1.074 | (0.97-1.18) |
| rs10206072 | 2 | 121373466 | G | A | 0.810 | 1.079 | (0.98-1.19) |
| rs16854905 | 2 | 169012955 | C | T | 0.832 | 0.999 | (0.90-1.11) |
| rs77167534 | 2 | 173319930 | C | T | 0.920 | 1.211 | (1.04-1.41) |
| rs34925593 | 2 | 174234547 | C | T | 0.487 | 1.058 | (0.98-1.14) |
| rs1861270 | 2 | 202126615 | G | A | 0.578 | 1.027 | (0.95-1.11) |
| rs12621900 | 2 | 208118301 | C | T | 0.688 | 1.037 | (0.95-1.13) |
| rs74001374 | 2 | 238411293 | C | T | 0.950 | 1.181 | (1.00-1.39) |
| rs2292884 | 2 | 238443226 | G | A | 0.284 | 1.016 | (0.94-1.10) |
| rs77559646 | 2 | 242135265 | A | G | 0.007 | 1.376 | (0.85-2.24) |
| rs77482050 | 2 | 242139600 | G | A | 0.995 | NA | (NA-NA) |
| rs2074840 | 2 | 242141719 | C | T | 0.526 | 1.077 | (1.00-1.16) |
| rs76832527 | 2 | 242157241 | A | G | 0.151 | 1.132 | (1.03-1.24) |
| rs6550597 | 3 | 18738940 | A | G | 0.699 | 1.096 | (1.01-1.19) |
| rs7618603 | 3 | 23153062 | A | C | 0.304 | 1.076 | (0.99-1.17) |
| rs34680713 | 3 | 49621718 | A | AT | 0.134 | 1.156 | (1.02-1.31) |
| rs13091518 | 3 | 70796696 | T | C | 0.714 | 1.039 | (0.95-1.13) |
| rs143745027 | 3 | 87144017 | G | A | 0.101 | 1.258 | (1.12-1.42) |
| rs7628934 | 3 | 87175984 | C | T | 0.615 | 1.077 | (1.00-1.16) |
| rs114810266 | 3 | 87399362 | A | G | 0.986 | 0.995 | (0.74-1.34) |
| rs1283104 | 3 | 106962521 | G | C | 0.491 | 1.027 | (0.95-1.10) |
| rs151038334 | 3 | 107193337 | C | T | 0.945 | 1.081 | (0.92-1.27) |
| rs2271494 | 3 | 113300183 | A | T | 0.634 | 1.033 | (0.96-1.12) |
| rs2811476 | 3 | 127898501 | C | A | 0.287 | 1.039 | (0.96-1.12) |
| rs35006112 | 3 | 128213994 | G | A | 0.744 | 1.079 | (0.99-1.17) |
| rs1457063 | 3 | 137562823 | A | G | 0.544 | 1.025 | (0.95-1.11) |
| rs7650602 | 3 | 141147414 | C | T | 0.433 | 0.969 | (0.90-1.05) |
| rs145053401 | 3 | 152011745 | C | CAT | 0.932 | 1.022 | (0.88-1.19) |
| rs2293607 | 3 | 169482335 | T | C | 0.601 | 1.032 | (0.96-1.11) |
| rs78416326 | 3 | 170074517 | G | C | 0.765 | 1.275 | (1.18-1.38) |
| rs577952184 | 3 | 170083540 | C | CTTTTT | 0.893 | 1.205 | (1.07-1.36) |
| rs11717887 | 3 | 172381777 | C | T | 0.370 | 0.949 | (0.88-1.03) |
| rs17804499 | 4 | 74442349 | G | C | 0.964 | 0.995 | (0.84-1.18) |
| rs13142786 | 4 | 74477135 | T | A | 0.514 | 1.049 | (0.97-1.13) |
| rs6853490 | 4 | 95544718 | G | A | 0.541 | 1.060 | (0.98-1.15) |
| rs7679673 | 4 | 106061534 | C | A | 0.471 | 1.072 | (1.00-1.15) |
| rs17035310 | 4 | 106064754 | C | T | 0.908 | 1.090 | (0.96-1.24) |
| rs77821238 | 4 | 140948835 | C | T | 0.895 | 1.009 | (0.89-1.14) |
| rs72725734 | 4 | 146879237 | G | A | 0.165 | 1.124 | (1.00-1.26) |
| rs147762399 | 4 | 152030340 | T | C | 0.029 | 0.961 | (0.78-1.19) |
| rs2242652 | 5 | 1280028 | G | A | 0.851 | 1.153 | (1.06-1.26) |
| rs71595003 | 5 | 1292118 | A | G | 0.005 | 1.097 | (0.60-1.99) |
| rs2736098 | 5 | 1294086 | T | C | 0.226 | 1.075 | (1.00-1.16) |
| rs4975758 | 5 | 1891174 | G | C | 0.499 | 1.070 | (1.00-1.15) |
| rs71599622 | 5 | 14372362 | T | TGA | 0.312 | 0.973 | (0.89-1.07) |
| rs10941370 | 5 | 37833419 | T | C | 0.630 | 1.025 | (0.95-1.11) |
| rs1482675 | 5 | 44368506 | T | C | 0.531 | 1.022 | (0.95-1.10) |
| rs9292122 | 5 | 56087910 | A | G | 0.475 | 1.087 | (1.01-1.17) |
| rs10793821 | 5 | 133836209 | T | C | 0.738 | 1.199 | (1.10-1.31) |
| rs76551843 | 5 | 169172133 | A | G | 0.999 | NA | (NA-NA) |
| rs9686557 | 5 | 172959030 | C | A | 0.403 | 1.031 | (0.95-1.11) |
| rs61739424 | 5 | 177683905 | G | A | 0.879 | 1.141 | (1.01-1.29) |
| rs2672843 | 5 | 177891551 | G | A | 0.284 | 1.057 | (0.98-1.14) |
| rs2814811 | 6 | 1670985 | A | G | 0.560 | 1.075 | (0.98-1.17) |
| rs2018336 | 6 | 11217897 | T | C | 0.830 | 1.066 | (0.96-1.18) |
| rs6927369 | 6 | 21330689 | C | T | 0.746 | 1.188 | (1.09-1.29) |
| rs4269363 | 6 | 21471490 | G | A | 0.495 | 1.015 | (0.95-1.09) |
| rs12665509 | 6 | 21878849 | A | C | 0.544 | 0.998 | (0.93-1.07) |
| rs35159226 | 6 | 26649830 | C | CT | 0.248 | 1.057 | (0.97-1.15) |
| rs62407547 | 6 | 30216712 | C | T | 0.200 | 1.046 | (0.91-1.20) |
| rs9275160 | 6 | 32652620 | A | G | 0.401 | 1.082 | (1.01-1.15) |
| rs9469899 | 6 | 34793124 | A | G | 0.309 | 1.015 | (0.94-1.10) |
| rs4714485 | 6 | 41536587 | G | T | 0.393 | 1.175 | (1.09-1.27) |
| rs9472120 | 6 | 43709785 | C | T | 0.441 | 1.034 | (0.96-1.12) |
| rs9443189 | 6 | 76495882 | A | G | 0.866 | 1.045 | (0.94-1.17) |
| rs2038542 | 6 | 109295293 | C | T | 0.106 | 1.026 | (0.91-1.16) |
| rs339351 | 6 | 117200434 | C | A | 0.726 | 0.997 | (0.92-1.08) |
| rs150184171 | 6 | 134292717 | G | GTGTTGT | 0.336 | 0.989 | (0.91-1.07) |
| rs13215045 | 6 | 153447516 | C | T | 0.706 | 1.057 | (0.97-1.15) |
| rs963800 | 6 | 160150279 | C | T | 0.770 | 1.084 | (1.00-1.18) |
| rs4646284 | 6 | 160581543 | TG | T | 0.230 | 1.146 | (1.06-1.24) |
| rs4513875 | 7 | 1928159 | T | C | 0.437 | 1.055 | (0.98-1.14) |
| rs11452686 | 7 | 20414110 | T | TA | 0.404 | 1.077 | (1.00-1.16) |
| rs9655205 | 7 | 20999211 | C | A | 0.132 | 1.075 | (0.98-1.18) |
| rs35389879 | 7 | 21812043 | T | G | 0.216 | 1.134 | (1.04-1.24) |
| rs6956484 | 7 | 27564862 | A | C | 0.462 | 1.041 | (0.96-1.13) |
| rs10486567 | 7 | 27976563 | G | A | 0.521 | 1.108 | (1.02-1.21) |
| rs12701838 | 7 | 40877473 | A | G | 0.774 | 1.049 | (0.96-1.15) |
| rs834608 | 7 | 47451918 | A | T | 0.585 | 1.054 | (0.98-1.14) |
| rs6955627 | 7 | 92577760 | C | T | 0.775 | 1.029 | (0.94-1.13) |
| rs4727386 | 7 | 97688440 | A | G | 0.670 | 1.136 | (1.05-1.23) |
| rs870167 | 8 | 8498803 | G | A | 0.264 | 1.070 | (0.98-1.17) |
| rs2572375 | 8 | 11217455 | C | T | 0.860 | 1.092 | (0.98-1.22) |
| rs6557704 | 8 | 23470785 | A | G | 0.225 | 1.075 | (0.99-1.17) |
| rs1160267 | 8 | 23529521 | G | A | 0.450 | 1.166 | (1.08-1.25) |
| rs141811748 | 8 | 25894201 | C | CCAAA | 0.164 | 1.066 | (0.96-1.18) |
| rs12677206 | 8 | 26063165 | A | C | 0.285 | 1.036 | (0.96-1.12) |
| rs11467 | 8 | 38644914 | A | G | 0.760 | 0.992 | (0.91-1.08) |
| rs73700335 | 8 | 108923107 | G | A | 0.888 | 0.970 | (0.86-1.09) |
| rs57588856 | 8 | 127836925 | C | CAA | 0.547 | 1.013 | (0.98-1.05) |
| rs6984837 | 8 | 127901649 | G | A | 0.369 | 1.069 | (0.98-1.16) |
| rs7011138 | 8 | 127922200 | A | T | 0.897 | 1.076 | (0.97-1.20) |
| rs7463326 | 8 | 128027954 | G | A | 0.697 | 1.103 | (1.00-1.21) |
| rs72725854 | 8 | 128074815 | T | A | 0.006 | NA | (NA-NA) |
| rs77541621 | 8 | 128077146 | A | G | 0.010 | 1.809 | (1.26-2.59) |
| rs72725879 | 8 | 128103969 | T | C | 0.243 | 1.209 | (1.01-1.45) |
| rs183373024 | 8 | 128104117 | G | A | 0.002 | NA | (NA-NA) |
| rs78809737 | 8 | 128104218 | G | A | 0.967 | 1.088 | (0.92-1.29) |
| rs201057014 | 8 | 128325355 | T | C | 0.112 | 1.054 | (0.98-1.13) |
| rs17464492 | 8 | 128342866 | A | G | 0.795 | 1.130 | (1.04-1.23) |
| rs6983267 | 8 | 128413305 | G | T | 0.603 | 1.210 | (1.12-1.31) |
| rs10090154 | 8 | 128532137 | T | C | 0.077 | 1.361 | (1.17-1.58) |
| rs34265760 | 8 | 128535543 | T | C | 0.922 | 1.181 | (1.01-1.38) |
| rs12549761 | 8 | 128540776 | C | G | 0.860 | 1.075 | (0.96-1.20) |
| rs34540271 | 9 | 18554773 | C | T | 0.611 | 1.064 | (0.99-1.15) |
| rs10122990 | 9 | 19072246 | C | A | 0.320 | 1.079 | (1.00-1.16) |
| rs17694493 | 9 | 22041998 | G | C | 0.075 | 1.067 | (0.93-1.22) |
| rs10122495 | 9 | 34049779 | T | A | 0.208 | 0.971 | (0.88-1.07) |
| rs142727307 | 9 | 82090723 | T | TG | 0.090 | 1.045 | (0.92-1.19) |
| rs4451364 | 9 | 109532734 | A | G | 0.862 | 1.071 | (0.97-1.19) |
| rs817872 | 9 | 110144887 | C | T | 0.302 | 1.119 | (1.04-1.21) |
| rs143655302 | 9 | 110290217 | G | A | 0.994 | NA | (NA-NA) |
| rs2241167 | 9 | 130430116 | A | G | 0.547 | 1.077 | (1.00-1.16) |
| rs12634 | 9 | 132573536 | T | G | 0.199 | 0.993 | (0.91-1.09) |
| rs12781100 | 10 | 838636 | T | C | 0.079 | 1.218 | (1.07-1.39) |
| rs7075427 | 10 | 46104943 | A | C | 0.920 | 1.120 | (0.96-1.31) |
| rs11599847 | 10 | 47599029 | T | C | 0.953 | 0.994 | (0.83-1.20) |
| rs10993994 | 10 | 51549496 | T | C | 0.349 | 1.291 | (1.19-1.39) |
| rs11817544 | 10 | 80236999 | C | A | 0.853 | 1.086 | (0.97-1.21) |
| rs12412705 | 10 | 80835998 | C | T | 0.208 | 1.026 | (0.94-1.12) |
| rs1935581 | 10 | 90195149 | C | T | 0.343 | 1.053 | (0.97-1.14) |
| rs12262998 | 10 | 104428716 | C | T | 0.767 | 1.167 | (1.07-1.28) |
| rs10885396 | 10 | 114711755 | T | C | 0.574 | 0.970 | (0.90-1.05) |
| rs4558107 | 10 | 122794926 | A | G | 0.264 | 1.007 | (0.93-1.09) |
| rs140783917 | 10 | 122834482 | C | T | 0.990 | 1.869 | (1.01-3.45) |
| rs10788167 | 10 | 123054018 | T | A | 0.760 | 1.043 | (0.96-1.14) |
| rs10749415 | 10 | 123185303 | A | G | 0.827 | 1.166 | (1.05-1.29) |
| rs12769682 | 10 | 126697494 | C | G | 0.199 | 1.034 | (0.94-1.13) |
| rs1881502 | 11 | 1507512 | T | C | 0.135 | 0.989 | (0.89-1.10) |
| rs11043143 | 11 | 2234093 | T | C | 0.308 | 1.128 | (1.04-1.22) |
| rs61890184 | 11 | 7547587 | A | G | 0.214 | 1.197 | (1.09-1.31) |
| rs68010938 | 11 | 47428209 | T | TA | 0.187 | 1.115 | (1.01-1.23) |
| rs1048374 | 11 | 58902679 | G | A | 0.069 | 1.093 | (0.94-1.28) |
| rs2277283 | 11 | 61908440 | C | T | 0.165 | 1.116 | (1.01-1.23) |
| rs12785905 | 11 | 66951965 | C | G | 0.024 | 1.318 | (1.01-1.72) |
| rs3018690 | 11 | 68882926 | T | C | 0.437 | 1.017 | (0.95-1.09) |
| rs11825796 | 11 | 68980788 | A | G | 0.185 | 1.062 | (0.97-1.16) |
| rs11228580 | 11 | 69002342 | C | T | 0.112 | 1.178 | (1.05-1.32) |
| rs3918298 | 11 | 69463273 | A | G | 0.041 | 1.377 | (1.21-1.57) |
| rs56159348 | 11 | 76267331 | T | G | 0.802 | 0.978 | (0.89-1.07) |
| rs11568818 | 11 | 102401661 | T | C | 0.650 | 1.157 | (1.07-1.25) |
| rs74911261 | 11 | 108357137 | A | G | 0.012 | 1.113 | (0.79-1.56) |
| rs5794883 | 11 | 113700546 | C | CA | 0.772 | 1.050 | (0.96-1.15) |
| rs138466039 | 11 | 125054793 | T | C | 0.004 | NA | (NA-NA) |
| rs878987 | 11 | 134266372 | G | A | 0.081 | 1.091 | (0.96-1.24) |
| rs2066827 | 12 | 12871099 | T | G | 0.819 | 0.975 | (0.89-1.07) |
| rs77216612 | 12 | 12877983 | A | G | 0.823 | 0.989 | (0.90-1.08) |
| rs10845938 | 12 | 14416918 | G | A | 0.629 | 1.086 | (1.00-1.18) |
| rs80130819 | 12 | 48419618 | A | C | 0.937 | 1.051 | (0.90-1.23) |
| rs56222401 | 12 | 49672714 | G | A | 0.293 | 1.030 | (0.95-1.12) |
| rs113925811 | 12 | 53308932 | A | C | 0.071 | 1.080 | (0.95-1.23) |
| rs187809440 | 12 | 53329231 | T | C | 0.006 | NA | (NA-NA) |
| rs7968403 | 12 | 65012824 | T | C | 0.722 | 1.007 | (0.93-1.10) |
| rs4842687 | 12 | 90156377 | A | G | 0.588 | 1.084 | (1.00-1.17) |
| rs77121786 | 12 | 102446675 | G | T | 0.121 | 1.034 | (0.92-1.16) |
| rs1270884 | 12 | 114685571 | A | G | 0.351 | 1.140 | (1.05-1.23) |
| rs7295014 | 12 | 133067989 | G | A | 0.464 | 1.023 | (0.95-1.10) |
| rs1327653 | 13 | 51076440 | T | C | 0.224 | 1.060 | (0.97-1.16) |
| rs7489409 | 13 | 73716861 | C | T | 0.374 | 1.036 | (0.96-1.12) |
| rs7336001 | 13 | 73995877 | G | C | 0.948 | 0.880 | (0.78-0.99) |
| rs75823044 | 13 | 110360784 | T | C | 0.002 | NA | (NA-NA) |
| rs1004030 | 14 | 23305649 | T | C | 0.760 | 1.082 | (0.99-1.18) |
| rs6571758 | 14 | 37136194 | G | A | 0.441 | 1.073 | (0.99-1.16) |
| rs11849126 | 14 | 38144592 | G | A | 0.753 | 0.966 | (0.89-1.05) |
| rs4901313 | 14 | 53387109 | G | T | 0.877 | 1.178 | (1.05-1.32) |
| rs8005621 | 14 | 61106699 | G | A | 0.108 | 1.117 | (0.99-1.26) |
| rs79133931 | 14 | 64687926 | T | C | 0.102 | 1.046 | (0.92-1.19) |
| rs2093202 | 14 | 68923908 | A | G | 0.719 | 1.136 | (1.05-1.23) |
| rs767127 | 14 | 69134264 | G | A | 0.560 | 1.009 | (0.94-1.09) |
| rs17565772 | 14 | 70756333 | G | A | 0.508 | 1.022 | (0.95-1.10) |
| rs11561564 | 15 | 40965044 | G | A | 0.586 | 1.042 | (0.96-1.13) |
| rs33984059 | 15 | 56385868 | A | G | 0.986 | 1.079 | (0.69-1.69) |
| rs74634457 | 15 | 66835704 | G | A | 0.149 | 1.138 | (1.04-1.25) |
| rs8023793 | 15 | 66942093 | A | C | 0.443 | 1.029 | (0.96-1.11) |
| rs12913603 | 15 | 70668824 | A | C | 0.494 | 1.038 | (0.96-1.12) |
| rs7188897 | 16 | 54469331 | T | C | 0.259 | 1.073 | (0.99-1.16) |
| rs13380763 | 16 | 54678305 | C | T | 0.894 | 1.001 | (0.90-1.12) |
| rs11863709 | 16 | 57654576 | C | T | 0.978 | 0.950 | (0.72-1.25) |
| rs28709974 | 16 | 79847632 | C | T | 0.041 | 1.102 | (0.91-1.33) |
| rs8052913 | 16 | 82166181 | C | T | 0.595 | 1.070 | (0.99-1.15) |
| rs684232 | 17 | 618965 | C | T | 0.408 | 1.121 | (1.04-1.21) |
| rs78378222 | 17 | 7571752 | G | T | 0.004 | NA | (NA-NA) |
| rs28441558 | 17 | 7803118 | C | T | 0.021 | 1.289 | (1.06-1.56) |
| rs72811270 | 17 | 12585459 | A | G | 0.170 | 1.019 | (0.92-1.13) |
| rs4795646 | 17 | 30092898 | G | A | 0.629 | 1.004 | (0.93-1.09) |
| rs3110641 | 17 | 36047417 | A | G | 0.220 | 1.080 | (1.00-1.17) |
| rs11649743 | 17 | 36074979 | G | A | 0.822 | 1.144 | (1.04-1.26) |
| rs11263763 | 17 | 36103565 | A | G | 0.607 | 1.159 | (1.08-1.25) |
| rs138213197 | 17 | 46805705 | T | C | 0.000 | NA | (NA-NA) |
| rs2960158 | 17 | 47380305 | T | C | 0.683 | 1.021 | (0.94-1.11) |
| rs565189650 | 17 | 47398245 | T | C | 0.041 | 1.053 | (0.89-1.25) |
| rs12938538 | 17 | 56426027 | T | C | 0.657 | 1.048 | (0.97-1.14) |
| rs148511027 | 17 | 69117532 | G | GTTAT | 0.587 | 1.181 | (1.09-1.27) |
| rs8089411 | 18 | 51771322 | C | T | 0.465 | 0.962 | (0.89-1.04) |
| rs35283980 | 18 | 56745999 | G | C | 0.463 | 1.065 | (0.99-1.15) |
| rs533722308 | 18 | 60961193 | CT | C | 0.400 | 1.048 | (0.95-1.15) |
| rs11876000 | 18 | 73035513 | T | G | 0.330 | 0.957 | (0.88-1.04) |
| rs9959454 | 18 | 76770820 | A | G | 0.736 | 1.125 | (1.03-1.23) |
| rs10412482 | 19 | 17228554 | C | T | 0.567 | 1.075 | (0.99-1.17) |
| rs17501397 | 19 | 32168343 | C | T | 0.957 | 1.078 | (0.89-1.31) |
| rs59710626 | 19 | 38548094 | G | T | 0.926 | 1.158 | (1.01-1.33) |
| rs4802297 | 19 | 38738130 | G | C | 0.418 | 1.082 | (1.01-1.16) |
| rs11673591 | 19 | 41985931 | T | A | 0.792 | 1.027 | (0.93-1.13) |
| rs2659051 | 19 | 51345568 | G | C | 0.724 | 1.106 | (1.02-1.20) |
| rs61752561 | 19 | 51361382 | G | A | 0.991 | 0.768 | (0.51-1.17) |
| rs76765083 | 19 | 51362715 | T | G | 0.961 | 1.271 | (1.04-1.55) |
| rs6039055 | 20 | 867324 | A | G | 0.758 | 0.982 | (0.90-1.08) |
| rs11480453 | 20 | 31347512 | C | CA | 0.406 | 0.988 | (0.91-1.07) |
| rs6141551 | 20 | 34006970 | C | T | 0.682 | 1.115 | (1.03-1.21) |
| rs73909841 | 20 | 49548807 | T | C | 0.922 | 0.978 | (0.85-1.12) |
| rs6126986 | 20 | 52464719 | C | T | 0.436 | 1.090 | (1.01-1.18) |
| rs139135938 | 20 | 61001061 | TGGCAGTGGCAGC | T | 0.543 | 1.033 | (0.96-1.12) |
| rs381331 | 20 | 62229989 | A | G | 0.499 | 1.061 | (0.99-1.14) |
| rs3787099 | 20 | 62307517 | A | G | 0.933 | 1.145 | (1.00-1.31) |
| rs1058319 | 20 | 62374389 | C | T | 0.800 | 1.082 | (0.99-1.19) |
| rs11701433 | 21 | 40296411 | C | T | 0.370 | 1.118 | (1.03-1.21) |
| rs61735792 | 21 | 42866332 | A | G | 0.018 | 1.228 | (0.95-1.60) |
| rs9978557 | 21 | 42882462 | C | T | 0.947 | 0.942 | (0.81-1.09) |
| rs1978060 | 22 | 19749525 | G | A | 0.496 | 1.062 | (0.98-1.15) |
| rs9625483 | 22 | 28888939 | A | G | 0.012 | 1.317 | (1.01-1.71) |
| rs17886163 | 22 | 29091147 | C | A | 0.002 | NA | (NA-NA) |
| rs555607708 | 22 | 29091856 | A | AG | 0.002 | NA | (NA-NA) |
| rs138708 | 22 | 39138332 | G | A | 0.986 | 1.331 | (0.94-1.88) |
| rs34584683 | 22 | 40499107 | T | A | 0.138 | 1.125 | (1.01-1.25) |
| rs6003062 | 22 | 43499741 | G | A | 0.944 | 1.156 | (0.97-1.37) |
| rs5759167 | 22 | 43500212 | G | T | 0.583 | 1.120 | (1.04-1.21) |
| rs9615099 | 22 | 45698149 | T | A | 0.655 | 1.094 | (1.01-1.19) |
| rs960417 | X | 9811095 | A | G | 0.875 | 1.016 | (0.94-1.10) |
| rs17321482 | X | 11482634 | C | T | 0.940 | 1.047 | (0.93-1.18) |
| rs5972255 | X | 30896320 | T | C | 0.390 | 1.034 | (0.98-1.09) |
| rs11338635 | X | 51245276 | GA | G | 0.188 | 1.031 | (0.97-1.10) |
| rs5943724 | X | 52695895 | G | A | 0.806 | 1.044 | (0.94-1.16) |
| rs4826594 | X | 54454406 | A | G | 0.269 | 1.023 | (0.96-1.09) |
| rs5919393 | X | 66825357 | T | C | 0.884 | 1.011 | (0.93-1.10) |
| rs371707439 | X | 70139908 | A | G | 0.131 | 0.993 | (0.89-1.10) |

Polymorphisms in linkage disequilibrium with those specifically mentioned herein are easily identified by those of skill in the art, such as using the HAPMAP database as described in WO 2016/049694.

### Ethnic Genotype Variation

It is known to those of skill in the art that genotypic variation exists between different populations. This phenomenon is referred to as human genetic variation. Human genetic variation is often observed between populations from different ethnic backgrounds. Such variation is rarely consistent and is often directed by various combinations of environmental and lifestyle factors. As a result of genetic variation, it is often difficult to identify a population of genetic markers such as polymorphisms that remain informative across various populations such as populations from different ethnic backgrounds.

A selection of polymorphisms that are common to at least four ethnic backgrounds and remain informative for assessing the risk for developing prostate cancer are disclosed herein (see Tables 1 to 4).

In an embodiment, the methods of the present disclosure can be used for assessing the risk for developing prostate cancer in human male subjects from various ethnic backgrounds. For example, the male subject can be classified as Caucasoid, Australoid, Mongoloid and Negroid based on physical anthropology. In particular, the inventors have found that the model can be used for Caucasians, African ancestry (including African Americans), East Asian ancestry and Hispanic ancestry.

In an embodiment, the subject is Caucasian.

In an embodiment, the subject is African.

In an embodiment, the subject is East Asian. In an embodiment, the East Asian subject is from China, Japan, South Korea, North Korea, Taiwan, Hong Kong, Mongolia or Macao.

In an embodiment, the subject is Hispanic.

It is well known that over time there has been blending of different ethnic origins. However, in practice this does not influence the ability of a skilled person to practice the invention.

A male subject of predominantly European origin, either direct or indirect through ancestry, with white skin is considered Caucasian in the context of the present disclosure. A Caucasian may have, for example, at least 75% Caucasian ancestry (for example, but not limited to, the male subject having at least three Caucasian grandparents).

A male subject of predominantly central or southern African origin, either direct or indirect through ancestry, is considered Negroid in the context of the present disclosure. A Negroid may have, for example, at least 75% Negroid ancestry. An American male subject with predominantly Negroid ancestry and black skin is considered African American in the context of the present disclosure. An African American may have, for example, at least 75% Negroid ancestry. Similar principle applies to, for example, males of Negroid ancestry living in other countries (for example Great Britain, Canada and The Netherlands).

A male subject predominantly originating from Spain or a Spanish-speaking country, such as a country of Central or Southern America, either direct or indirect through ancestry, is considered Hispanic in the context of the present disclosure. A Hispanic may have, for example, at least 75% Hispanic ancestry.

The terms “ethnicity” and “race” can be used interchangeably in the context of the present disclosure. In an embodiment, the genetic risk assessment can readily be practiced based on what ethnicity the subject considers themselves to be. Thus, in an embodiment, the ethnicity of the human male subject is self-reported by the subject. As an example, male subjects can be asked to identify their ethnicity in response to this question: “To what ethnic group do you belong?”. In another example, the ethnicity of the male subject is derived from medical records after obtaining the appropriate consent from the subject or from the opinion or observations of a clinician.

### Genetic Risk Score

In an embodiment, the genetic risk assessment involves determining a polygenic risk score for the subject (also referred to herein as PRS or “genetic risk score”). An individual's PRS can be defined as the weighted sum of the individuals' genotypes at multiple genetic loci. In other words, they are the linear combinations of the risk alleles across a set of candidate polymorphisms.

In one embodiment, the key steps to construct a polygenic risk score (PRS) are to determine which polymorphisms to include and how to weight their effects. In one embodiment, the maxCT and SCT methods (Privé et al., 2019) are used. These methods are based on clumping and thresholding. The aim of clumping and thresholding is to remove correlated polymorphisms while keeping the most important polymorphisms in the PRS. To do this, the values of a range of hyperparameters including the correlation threshold (r²), the clumping window size (kb) and the p-value significance threshold (p) are decided. Different selection of these hyperparameters values would in general give a different section of polymorphisms to include. For the weights, reported GWAS coefficients (i.e., regression coefficients or log odds ratios) from external published GWAS can be used. The core idea of the maxCT and SCT procedures is to select a set of different hyperparameters values and compute a PRS for each combination of these values.

This will usually create a large number of PRSs, for example, around 100,000 vectors of PRSs for a typical GWAS. After constructing these RPSs, there are two approaches to create the final PRS. In maxCT, the PRS that has the strongest predictive performance (e.g., largest AUC) are selected as the final PRS. In SCT, the PRSs by a penalized logistic regression model, for example, the popular lasso procedure, are combined. Since the outcome of SCT will be a linear combination of PRSs, where each PRS is again a linear combination of variants, the final PRS still has the form of (1), which means the effect sizes of polymorphisms can be obtained and used for prediction.

In a preferred embodiment, a log-additive risk model can be used to define three genotypes AA, AB, and BB for a single polymorphism having relative risk values of 1, OR, and OR², under a rare disease model, where OR is the previously reported disease odds ratio for the effect allele, B, vs the reference allele, A. If the B allele has frequency (p), then these genotypes have population frequencies of (1−p)², 2p(1−p), and p², assuming Hardy-Weinberg equilibrium. The relative risk values for each polymorphism can then be scaled so that based on these frequencies the average relative risk in the population is 1 (Mealiffe et al., 2010). Specifically, given the unscaled population average relative risk for each SNP is:

$$μ = {( 1 - p )}^{2} + 2 p ( 1 - p ) O R + {p}^{2} O {R}^{2}$$

Adjusted risk values 1/μ, OR/μ, and OR²/μ are used for the AA, AB, and BB genotypes, respectively. Missing genotypes are assigned an adjusted risk of 1. The final PRS is obtained by multiplying the adjusted risk values for each SNP.

Similar calculations can be performed for non-SNP polymorphisms.

It is envisaged that the risk of a human subject for developing prostate cancer can be provided as a relative risk or an absolute risk as required. In an embodiment, as with the above example, the genetic risk assessment obtains the absolute risk of a human subject for developing prostate cancer. Absolute risk is the numerical probability of a human subject developing prostate cancer within a specified period (e.g. 5, 10, 15, 20 or more years). It reflects a human subject's risk of developing prostate cancer insofar as it does not consider various risk factors in isolation.

In an alternate embodiment, the genetic risk assessment obtains the relative risk of a human subject for developing prostate cancer. Relative risk, measured as the incidence of a disease in individuals with a particular characteristic (or exposure) divided by the incidence of the disease in individuals without the characteristic, indicates whether that particular exposure increases or decreases risk. Relative risk is helpful to identify characteristics that are associated with a disease, but by itself is not particularly helpful in guiding screening decisions because the frequency of the risk (incidence) is cancelled out.

An alternate method for calculating the composite polymorphism risk is described in Mavaddat et al. (2015). In this example, the following formula is used;

$$PRS = {β}_{1} {x}_{1} + {β}_{2} {x}_{2} + … {β}_{κ} {x}_{κ} + {β}_{n} {x}_{n}$$

where β_{κ} is the per-allele log odds ratio (OR) for prostate cancer associated with the minor allele for polymorphisms κ, and x_{κ} the number of alleles for the same polymorphisms (0, 1 or 2), n is the total number of polymorphism and PRS is the polygenic risk score (which can also be referred to as composite polymorphism risk).

### Prostate Cancer Risk Assessment

As the skilled person would be aware, in view of the teachings of the present disclosure, a variety of different formulae could be produced to provide a risk score.

In an embodiment, the polygenic risk score is produced using a formula as described above, preferably using

$$μ = {( 1 - p )}^{2} + 2 p ( 1 - p ) O R + {p}^{2} O {R}^{2}$$

Adjusted risks (which have a population average risk equal to 1) for each polymorphism are calculated as adjusted_risk=OR^{N} divided by u, where N is the number of risk alleles.

The overall PRS (such as snp269 when the polymorphisms in Table 1 are used) is then the product of the adjusted risk values for each of the polymorphisms.

The respective odds ratios (OR) (see, for example, Tables 1 to 4) for each possible genotype (AA, AB, BB) for each polymorphism, calculated using the above methodology used to calculate the PRS. The natural logarithm of the PRS is used in the risk equations.

In an embodiment, relative risk (rrisk) of developing prostate cancer is determined using;

$$rrisk = {e}^{\begin{matrix}( PDCE 1 \times lnprs + PDCE 2 \times fh 1 + PDCE 3 \times fh 2 + PDCE 4 \times agegp + \\ PDCE 5 \times age \times lnprs + P DCE 6 \times age \times fh 1 + PDCE 7 \times age \times fh 2 )\end{matrix}}$$

PDCE1 is a predetermined β coefficient for the natural logarithm of a PRS,
PDCE2 is a predetermined β coefficient if the subject has at least one first-degree relative with, or who has had, prostate cancer,
PDCE3 is a predetermined β coefficient if the subject has two or more first-degree relatives with, or who have had, prostate cancer,
PDCE4 is a predetermined β coefficient for age category,
PDCE5 is a predetermined β coefficient for the interaction between age in years and the PRS,
PDCE6 is a predetermined β coefficient based on the interaction between age in years and if the subject has at least one first-degree relative with, or who has had, prostate cancer,
PDCE7 is a predetermined β coefficient based on the interaction between age in years and if the subject has two or more first-degree relatives with, or who have had, prostate cancer,
lnprs is the natural logarithm of the PRS,
fh1 is 1 if the subject has at least one first-degree relative with, or who has had, prostate cancer, and 0 if not,
fh2 is 1 if the subject has two or more first-degree relatives with, or who have had, prostate cancer, and 0 if not,
age is the age of the subject in years.

In an embodiment, the age categories are 40 to 49, 50 to 59 and 60 to 69. In an embodiment,
i) if the subject is 40 to 49 years of age the value is 1,
ii) if the subject is 50 to 59 years of age the value is 2, and
iii) if the subject is 60 to 69 years of age the value is 3.

In an embodiment, PDCE1 is between 1.266 and 2.266.

In an embodiment, PDCE2 is between 1.716 and 2.716.

In an embodiment, PDCE3 is between 4.084 and 6.084.

In an embodiment, PDCE4 is between 0.022 and 0.082.

In an embodiment, PDCE5 is between −0.003 and −0.023.

In an embodiment, PDCE6 is between −0.017 and −0.037.

In an embodiment, PDCE7 is between −0.035 and −0.095.

In an embodiment, the β coefficients outlined in Table 5 are used for prostate cancer risk assessment.

TABLE 5
Example of Clinical Risk Factors

| Variable | Variable name | Coefficient |
|---|---|---|
| In(PRS) | Inprs | 1.766 |
| Number of affected | | |
| first-degree relatives | | |
| 0 | fh0 (0 = no, 1 = yes) | — |
| 1 | fh1 (0 = no, 1 = yes) | 2.216 |
| 2 | fh2 (0 = no, 1 = yes) | 5.084 |
| 10-year age group | agegp (1 = 40-49, | 0.052 |
| | 2 = 50-59, 3 = 60-69) | |
| Age (years) by In(PRS) | age × Inprs | −0.013 |
| Age (years) by number | | |
| of affected first- | | |
| degree relatives | | |
| 0 | age × fh0 (0 = no, | — |
| | 1 = yes) | |
| 1 | age × fh1 (0 = no, | −0.027 |
| | 1 = yes) | |
| 2 | age × fh2 (0 = no, | −0.065 |
| | 1 = yes) | |

In an embodiment, one or more or all of the β coefficients in Table 5 can be varied by to ±10%, more preferably ±5%, more preferably ±1%, of the designated value.

In an embodiment, the last or second last decimal point can be removed and the relevant number rounded up (from 5) or down (from 4).

In an embodiment, the risk of developing prostate cancer is determined using:

$$rrisk = {e}^{\begin{matrix}( 1.766 \times lnprs + 2.216 \times fh 1 + 5.084 \times fh 2 + 0.052 \times agegp - \\ 0.013 \times age \times lnprs - 0.027 \times age \times fh 1 - 0.065 \times age \times fh 2 )\end{matrix}}$$

For example, a 57-year-old man with one affected first-degree relative and a PRS of 1.10 (i.e. lnprs=0.0953) will have:

$$rrisk = {e}^{( 1.766 \times 0.0953 + 2.216 \times 1 + 5.084 \times 0 + 0.052 \times 2 - 0.013 \times 57 \times 0.0953 - 0.027 \times 57 \times 1 - 0.065 \times 57 \times 0 )}$$

$$rrisk = {e}^{0.8787}$$

$$rrisk = 2.4 077$$

In an alternate embodiment, relative risk (rrisk) of developing prostate cancer is determined using;

$$rrisk = {e}^{( P DCE 1 \times lnps + PDCE 2 \times fh 1 + PDCE 3 \times fh 2 + PDCE 4 \times age 1 + P DCE 5 \times age 2 + P DCE 6 \times ( age - 55 ) \times lnp r s}$$

where;
PDCE1 is a predetermined β coefficient for the natural logarithm of a PRS,
PDCE2 is a predetermined β coefficient if the subject has at least one first-degree relative with, or who has had, prostate cancer,
PDCE3 is a predetermined β coefficient if the subject has two or more first-degree relatives with, or who have had, prostate cancer,
PDCE4 is a predetermined β coefficient if the subject is 50 to 59 years of age,
PDCE5 is a predetermined β coefficient if the subject is 60 to 69 years of age,
PDCE6 is a predetermined β coefficient based on an interaction between the age of the subject and the natural logarithm of the PRS,
lnprs is the natural logarithm of the PRS
fh1 is 1 if the subject has at least one first-degree relative with, or who has had, prostate cancer, and 0 if not,
fh2 is 1 if the subject has two or more first-degree relatives with, or who have had, prostate cancer, and 0 if not,
age1 is 1 if the subject is 50 to 59 years of age and 0 if not,
age2 is 1 if the subject is 60 to 69 years of age and 0 if not,
age is the age of the subject in years.

In an embodiment, PDCE1 is between 0.913 and 1.113.

In an embodiment, PDCE2 is between 0.327 and 0.527.

In an embodiment, PDCE3 is between 0.737 and 0.937.

In an embodiment, PDCE4 is between 0.023 and 0.223.

In an embodiment, PDCE5 is between 0.177 and 0.377.

In an embodiment, PDCE6 is between −0.089 and 0.111.

In an embodiment, the β coefficients outlined in Table 6 are used for prostate cancer risk assessment.

TABLE 6
Another Example of Clinical Risk Factors

| Variable | Variable name | Coefficient |
|---|---|---|
| In(PRS) | Inprs | 1.013 |
| Number of affected | | |
| first-degree relatives | | |
| 0 | fh0 (0 = no, | 0 |
| | 1 = yes) | |
| 1 | fh1 (0 = no, | 0.427 |
| | 1 = yes) | |
| 2 | fh2 (0 = no, | 0.837 |
| | 1 = yes) | |
| 10-year age group | | |
| 40-49 years | | 0 |
| 50-59 years | | 0.123 |
| 60-69 years | | 0.277 |
| Age (years) and | (age-55) × | 0.011 |
| In(PRS) interaction | In(PRS) | |

In an embodiment, one or more or all of the β coefficients in Table 6 can be varied by to ±10%, more preferably ±5%, more preferably ±1%, of the designated value.

In an embodiment, the last or second last decimal point can be removed and the relevant number rounded up (from 5) or down (from 4).

Thus, in an alternate embodiment the risk of developing prostate cancer is determined using:

$$rrisk = {e}^{( 1.013 \times lnps + 0.427 \times fh 1 + 0.837 \times fh 2 + 0.123 \times age 1 + 0.277 \times age 2 + 0.011 \times ( age - 55 ) \times lnp r s}$$

For example, a 57-year-old man with one affected first-degree relative and a PRS of 1.10 (i.e. lnPRS=0.0953) will have:

$$rrisk = {e}^{( 1.013 \times 0.0953 + 0.427 \times 1 + 0.837 \times 0 + 0.123 \times 1 + 0.277 \times 0 + 0.011 \times ( 57 - 55 ) \times 0.0953 )}$$

$$rrisk = {e}^{0.6486}$$

$$rrisk = 1.9129$$

The subject's results can be one or more or all of their absolute 5-year absolute risk, 10-year absolute risk and remaining lifetime risk up to age 90 years, which can be calculated as defined below.

### Cumulative Risks

$$cumul\_b = 1 - {e}^{- r r isk \times incid \_ b}$$

$$cumul\_b \_ 5 = 1 - {e}^{- r r isk \times incid \_ b \_ 5}$$

$$cumul\_b \_ 10 = 1 - {e}^{- r risk \times incid \_ b \_ 10}$$

$$cumul\_full \_life = 1 - {e}^{- r r i s k \times incid \_ life}$$

### Absolute 5-Year Risk

$$pr\_risk \_ 5 yr = \frac{( cumul\_b \_ 5 - cumul\_b )}{( 1 - cumul\_b )}$$

### Absolute 10-Year Risk

$$pr\_risk \_ 10 yr = \frac{( cumul\_b \_ 10 - cumul\_b )}{( 1 - cumul\_b )}$$

### Absolute Remaining Lifetime Risk (to Age 90)

$$pr\_risk \_rem \_life = \frac{( cumul\_full \_life - cumul\_b )}{( 1 - cumul\_b )}$$

The subject's results can be one or more or all of their population average 5-year risk, population average 10-year risk and population average remaining lifetime risk up to 90 years which can be calculated as defined below.

### Cumulative Risks

$$cumul\_av \_b = 1 - {e}^{- incid \_ b}$$

$$cumul\_av \_b \_ 5 = 1 - {e}^{- incid \_ b \_ 5}$$

$$cumul\_av \_b \_ 10 = 1 - {e}^{- incid \_ b \_ 10}$$

$$cumul\_av \_full \_life = 1 - {e}^{- incid \_ life}$$

### Population Average 5-Year Risk

$$risk\_av \_ 5 = \frac{( cumul\_av \_b \_ 5 - cumul\_av \_b )}{( 1 - cumul\_av \_b )}$$

### Population Average 10-Year Risk

$$risk\_av \_ 10 = \frac{( cumul\_av \_b \_ 10 - cumul\_av \_b )}{( 1 - cumul\_av \_b )}$$

### Population Average Remaining Lifetime Risk (to Age 90)

$$risk\_av \_rem \_life = \frac{( cumul\_av \_full \_life - cumul\_av \_b )}{( 1 - cumul\_av \_b )}$$

In an embodiment, one or more threshold value(s) are set for determining a particular action such as the need for routine diagnostic testing or preventative therapy. For example, a score determined using a method of the invention is compared to a pre-determined threshold, and if the score is higher than the threshold a recommendation is made to take the pre-determined action. Methods of setting such thresholds have now become widely used in the art and are described in, for example, US20140018258.

### Treatment

After performing the methods of the present disclosure treatment may be prescribed or administered to the subject.

Accordingly, in an embodiment, the methods of the present disclosure relate to an anti-cancer therapy for use in preventing or reducing the risk of prostate cancer in a human subject at risk thereof.

One of skill in the art will appreciate that prostate cancer is a heterogeneous disease with distinct clinical outcomes. In one embodiment, it is not envisaged that the methods of the present disclosure be limited to assessing the risk of developing a particular type or subtype of prostate cancer.

In one embodiment, the therapy is radiation therapy such as external-beam radiation therapy, brachytherapy, intensity-modulated radiation therapy (IMRT), or proton therapy.

In one embodiment, the therapy is hormonal therapy such as therapies which lower androgen levels, in particular lower testosterone levels. Examples of such therapies include, but are not limited to, LHRH agonists, LHRH antagonists, androgen receptor (AR) inhibitors such as Apalutamide, Darolutamide or Enzalutamide, androgen synthesis inhibitors such as abiraterone acetate or ketoconazole, PARP inhibitors such as Olaparib (Lynparza) or Rucaparib (Rubraca), or immunotherapy such as CAR-T therapy.

In an embodiment, the methods of the present disclosure are used to assess the risk of a human male subject for developing prostate cancer and administering a treatment appropriate for the risk of developing prostate cancer. For example, when performing the methods of the present disclosure indicates a high risk of prostate cancer an aggressive chemopreventative treatment regimen can be established. In contrast, when performing the methods of the present disclosure indicates a moderate risk of prostate cancer a less aggressive chemopreventative treatment regimen can be established. Alternatively, when performing the methods of the present disclosure indicates a low risk of prostate cancer a chemopreventative treatment regimen need not be established. It is envisaged that the methods of the present disclosure can be performed over time so that the treatment regimen can be modified in accordance with the subject's risk of developing prostate cancer.

### Marker Detection Strategies

Amplification primers for amplifying markers (e.g., marker loci) and suitable probes to detect such markers or to genotype a sample with respect to multiple marker alleles, can be used in the disclosure. For example, primer selection for long-range PCR is described in U.S. Ser. No. 10/042,406 and U.S. Ser. No. 10/236,480; for short-range PCR, U.S. Ser. No. 10/341,832 provides guidance with respect to primer selection. Also, there are publicly available programs such as Oligo available for primer design. With such available primer selection and design software, the publicly available human genome sequence and the polymorphism locations, one of skill can construct primers to amplify the polymorphisms to practice the disclosure. Further, it will be appreciated that the precise probe to be used for detection of a nucleic acid comprising a polymorphism (e.g., an amplicon comprising the polymorphism) can vary, e.g., any probe that can identify the region of a marker amplicon to be detected can be used in conjunction with the present disclosure. Further, the configuration of the detection probes can, of course, vary. Thus, the disclosure is not limited to the sequences recited herein.

Indeed, it will be appreciated that amplification is not a requirement for marker detection, for example one can directly detect unamplified genomic DNA simply by performing a Southern blot on a sample of genomic DNA.

Typically, molecular markers are detected by any established method available in the art, including, without limitation, ASH, detection of extension, array hybridization (optionally including ASH), or other methods for detecting polymorphisms, AFLP detection, amplified variable sequence detection, randomly amplified polymorphic DNA (RAPD) detection, RFLP detection, self-sustained sequence replication detection, SSR detection, and single-strand conformation polymorphisms (SSCP) detection.

As the skilled person will appreciate, the sequence of the genomic region to which these oligonucleotides hybridize can be used to design primers which are longer at the 5′ and/or 3′ end, possibly shorter at the 5′ and/or 3′ (as long as the truncated version can still be used for amplification), which have one or a few nucleotide differences (but nonetheless can still be used for amplification), or which share no sequence similarity with those provided but which are designed based on genomic sequences close to where the specifically provided oligonucleotides hybridize and which can still be used for amplification.

In some embodiments, the primers are radiolabelled, or labelled by any suitable means (e.g., using a non-radioactive fluorescent tag), to allow for rapid visualization of differently sized amplicons following an amplification reaction without any additional labelling step or visualization step. In some embodiments, the primers are not labelled, and the amplicons are visualized following their size resolution, e.g., following agarose or acrylamide gel electrophoresis. In some embodiments, ethidium bromide staining of the PCR amplicons following size resolution allows visualization of the different size amplicons.

It is not intended that the primers be limited to generating an amplicon of any particular size. For example, the primers used to amplify the marker loci and alleles herein are not limited to amplifying the entire region of the relevant locus, or any subregion thereof. The primers can generate an amplicon of any suitable length for detection. In some embodiments, marker amplification produces an amplicon at least 20 nucleotides in length, or alternatively, at least 50 nucleotides in length, or alternatively, at least 100 nucleotides in length, or alternatively, at least 200 nucleotides in length. Amplicons of any size can be detected using the various technologies described herein. Differences in base composition or size can be detected by conventional methods such as electrophoresis.

Some techniques for detecting genetic markers utilize hybridization of a probe nucleic acid to nucleic acids corresponding to the genetic marker (e.g., amplified nucleic acids produced using genomic DNA as a template). Hybridization formats, including, but not limited to: solution phase, solid phase, mixed phase, or in situ hybridization assays are useful for allele detection. An extensive guide to the hybridization of nucleic acids is found in Tijssen (1993) Laboratory Techniques in Biochemistry and Molecular Biology—Hybridization with Nucleic Acid Probes Elsevier, New York, as well as in Sambrook et al. (supra).

PCR detection using dual-labelled fluorogenic oligonucleotide probes, commonly referred to as “TaqMan™” probes, can also be performed according to the present disclosure. These probes are composed of short (e.g., 20-25 base) oligodeoxynucleotides that are labelled with two different fluorescent dyes. On the 5′ terminus of each probe is a reporter dye, and on the 3′ terminus of each probe a quenching dye is found. The oligonucleotide probe sequence is complementary to an internal target sequence present in a PCR amplicon. When the probe is intact, energy transfer occurs between the two fluorophores and emission from the reporter is quenched by the quencher by FRET. During the extension phase of PCR, the probe is cleaved by 5′ nuclease activity of the polymerase used in the reaction, thereby releasing the reporter from the oligonucleotide-quencher and producing an increase in reporter emission intensity. Accordingly, TaqMan™ probes are oligonucleotides that have a label and a quencher, where the label is released during amplification by the exonuclease action of the polymerase used in amplification. This provides a real time measure of amplification during synthesis. A variety of TaqMan™ reagents are commercially available, e.g., from Applied Biosystems (Division Headquarters in Foster City, Calif.) as well as from a variety of specialty vendors such as Biosearch Technologies (e.g., black hole quencher probes). Further details regarding dual-label probe strategies can be found, e.g., in WO 92/02638.

Other similar methods include e.g. fluorescence resonance energy transfer between two adjacently hybridized probes, e.g., using the “LightCycler®” format described in U.S. Pat. No. 6,174,670.

Array-based detection can be performed using commercially available arrays, e.g., from Affymetrix (Santa Clara, Calif.) or other manufacturers. Reviews regarding the operation of nucleic acid arrays include Sapolsky et al. (1999); Lockhart (1998); Fodor (1997a); Fodor (1997b) and Chee et al. (1996). Array based detection is one preferred method for identification markers of the disclosure in samples, due to the inherently high-throughput nature of array based detection.

The nucleic acid sample to be analysed is isolated, amplified and, typically, labelled with biotin and/or a fluorescent reporter group. The labelled nucleic acid sample is then incubated with the array using a fluidics station and hybridization oven. The array can be washed and or stained or counter-stained, as appropriate to the detection method. After hybridization, washing and staining, the array is inserted into a scanner, where patterns of hybridization are detected. The hybridization data are collected as light emitted from the fluorescent reporter groups already incorporated into the labelled nucleic acid, which is now bound to the probe array. Probes that most clearly match the labelled nucleic acid produce stronger signals than those that have mismatches. Since the sequence and position of each probe on the array are known, by complementarity, the identity of the nucleic acid sample applied to the probe array can be identified.

Markers and polymorphisms can also be detected using DNA sequencing. DNA sequencing methods are well known in the art and can be found for example in Ausubel et al, eds., Short Protocols in Molecular Biology, 3rd ed., Wiley, (1995) and Sambrook et al, Molecular Cloning, 2nd ed., Chap. 13, Cold Spring Harbor Laboratory Press, (1989). Sequencing can be carried out by any suitable method, for example, dideoxy sequencing, chemical sequencing, or variations thereof.

Suitable sequencing methods also include Second Generation, Third Generation, or Fourth Generation sequencing technologies, all referred to herein as “next generation sequencing”, including, but not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology, etc. A review of some such technologies can be found in (Morozova and Marra, 2008), herein incorporated by reference. Accordingly, in some embodiments, performing a genetic risk assessment as described herein involves detecting the at least two polymorphisms by DNA sequencing. In an embodiment, the at least two polymorphisms are detected by next generation sequencing.

Next generation sequencing (NGS) methods share the common feature of massively parallel, high-throughput strategies, with the goal of lower costs in comparison to older sequencing methods (see Voelkerding et al., 2009; MacLean et al., 2009).

A number of such DNA sequencing techniques are known in the art, including fluorescence-based sequencing methodologies (Birren et al., 1997). In some embodiments, automated sequencing techniques are used. In some embodiments, parallel sequencing of partitioned amplicons is used (WO2006084132). In some embodiments, DNA sequencing is achieved by parallel oligonucleotide extension (See, e.g., U.S. Pat. Nos. 5,750,341 and 6,306,597). Additional examples of sequencing techniques include the Church polony technology (Mitra et al., 2003; Shendure et al., 2005; US 6,432,36; U.S. Pat. Nos. 6,485,944; 6,511,803), the 454 picotiter pyrosequencing technology (Margulies et al., 2005; US20050130173), the Solexa single base addition technology (Bennett et al., 2005; U.S. Pat. Nos. 6,787,308; 6,833,246), the Lynx massively parallel signature sequencing technology (Brenner et al., 2000; U.S. Pat. Nos. 5,695,934; 5,714,330), and the Adessi PCR colony technology (Adessi et al., 2000).

### Computer-Implemented Method

It is envisaged that the methods of the present disclosure may be implemented by a system such as a computer-implemented method. For example, the system may be a computer system comprising one or a plurality of processors which may operate together (referred to for convenience as “processor”) connected to a memory. The memory may be a non-transitory computer readable medium, such as a hard drive, a solid state disk, CD-ROM or the cloud. Software, that is executable instructions or program code, such as program code grouped into code modules, may be stored on the memory, and may, when executed by the processor, cause the computer system to perform functions such as determining that a task is to be performed to assist a user to determine the risk of a human male subject for developing prostate cancer; receiving data relating to one or more clinical factors as discussed herein, receiving data relating to the genetic risk assessment, wherein the genetic risk was derived by detecting at least two polymorphisms known to be associated with prostate cancer; processing the data to obtain the risk of a human male subject for developing prostate cancer; outputting the risk of a human male subject for developing prostate cancer.

For example, the memory may comprise program code, which when executed by the processor causes the system to determine at least two polymorphisms known to be associated with prostate cancer; process the data to combine clinical and genetic risk assessments to obtain the risk of a human male subject for developing prostate cancer; report the risk of a human male subject for developing prostate cancer.

In another embodiment, the system may be coupled to a user interface to enable the system to receive information from a user and/or to output or display information. For example, the user interface may comprise a graphical user interface, a voice user interface or a touchscreen.

In an embodiment, the program code may cause the system to determine the “Prostate Cancer Risk”.

In an embodiment, the system may be configured to communicate with at least one remote device or server across a communications network such as a wireless communications network. For example, the system may be configured to receive information from the device or server across the communications network and to transmit information to the same or a different device or server across the communications network. In other embodiments, the system may be isolated from direct user interaction.

In another embodiment, the diagnostic or prognostic rule is based on the application of a statistical and machine learning algorithm. Such an algorithm uses relationships between a population of polymorphisms and disease status observed in training data (with known disease status) to infer relationships which are then used to determine the risk of a human male subject for developing prostate cancer in subjects with an unknown risk. An algorithm is employed which provides a risk of a human male subject developing prostate cancer. The algorithm performs a multivariate or univariate analysis function.

### EXAMPLES

### Example 1—Materials and Methods

The inventors used data from the UK Biobank for these analyses. The UK Biobank is a population-based cohort of over 500 000 participants who were recruited from England, Wales and Scotland from 2006 to 2009 (Bycroft et al, 2018; Sudlow et al., 2015). At the baseline assessment, UK Biobank staff collected extensive phenotypic information from participants using a touch-screen questionnaire, face-to-face interview and by taking physical measurements. Biological samples (blood, urine and saliva) collected at the baseline assessment have provided genomic and biomarker information for almost all participants.

Extensive outcome data is provided through linkage to cancer registries (complete to 31 Jul. 2019 for England and Wales, and complete to 31 Oct. 2015 for Scotland), death registries (complete to 28 Feb. 2021 for all), hospital data (complete to 31 Mar. 2021 for England and Scotland, and complete to 28 Feb. 2018 for Wales) and primary care data (complete to 31 May 2016 for England, 31 Mar. 2017 for Scotland and 31 Aug. 2017 for Wales for approximately 45% of participants) (Bycroft et al, 2018; Sudlow et al., 2015; UK Biobank, 2021).

There is evidence of a healthy volunteer selection bias in the UK Biobank, and therefore, the participants are not representative of the general population (Fry et al., 2017). Compared with non-participants, UK Biobank participants were more likely to be older, female and not live in socioeconomically deprived regions. Participants were also found to be healthier than the general population. Despite this, the size of the UK Biobank and the variation in exposure measures mean that analyses of exposures and disease outcomes do not require representative data to be generalisable to other populations (Fry et al., 2017).

Eligible participants were active UK Biobank members (at 22 Feb. 2022) who were male, genetically Caucasian and aged 40 to 69 years at their baseline assessment date. The inventors excluded men who had prostate cancer diagnosed before their baseline assessment date, did not have SNP data available, or had died or been diagnosed with prostate cancer within the first six weeks of follow-up. The inventors then limited the dataset to unrelated men by using the ukb_gen_samples_to_remove function of the R package ukbtools (Handscomb et al., 2019) to ensure that there were no pairs of men with closer than third-degree relatedness. There were 189 338 men in the final analysis dataset, 8996 of whom had incident prostate cancer. Details of the eligibility criteria and number of men dropped at each step are shown in Table 7.

TABLE 7
Eligibility criteria for the current study.

| N eligible | Criteria | N dropped |
|---|---|---|
| 502 413 | Active UK Biobank participants | |
| | (at 22 Feb. 2022) | |
| 228 850 | Male and reported sex same as genetic sex | 273 185 |
| 227 257 | Aged 40-69 years at baseline assessment date | 1593 |
| 213 489 | White British and genetically Caucasian | 13 768 |
| 210 107 | No prostate cancer at baseline assessment date | 3382 |
| 204 918 | Genotyping data available | 5189 |
| 204 902 | Alive after six weeks of follow-up | 16 |
| 204 832 | No prostate cancer after six weeks of follow-up | 70 |
| 198 334 | Unrelated individuals (≥3rd | 6498 |
| | degree relatedness) | |

Age at baseline assessment was obtained from UK Biobank data field 21003. Prostate cancer was identified based on self-reported data (UK Biobank data field 20001 equal to 1044) and linked cancer registry data (the first three digits of UK Biobank data field 40006 [ICD9] equal to 185 or the first three characters of UK Biobank data field 40006 [ICD10] equal to C61). Incident prostate cancers were those for which the date of diagnosis (UK Biobank data field 40005) was after the baseline assessment date (UK Biobank data field 53), while prevalent prostate cancers were those diagnosed on or before the baseline assessment date (UK Biobank data fields 20007 or 40005 less than or equal to baseline assessment date).

The inventors used linked death registry data (UK Biobank data fields 40000 [date of death] and 40007 [age at death]) to identify men who had died during the follow-up period. A first-degree family history of prostate cancer was ascertained where UK Biobank data fields 20107 (illnesses of father) and 20111 (illnesses of siblings) were equal to 13. Because of the way the questions were worded, the inventors were not able to identify participants with more than one affected brother. Therefore, the number of participants with two affected first-degree relatives will only reflect those with both an affected father and at least one affected brother.

The inventors used Plink version 1.9 (Purcell and Chang, 2021; Chang et al., 2015) to extract the genotypes of 264 of the 269 SNPs identified by Conti et al. (2021) from the UK Biobank imputation dataset. Of the five missing SNPs, the inventors were unable to find three (rs35159226, rs150184171 and rs11338635) in the imputation dataset, one (rs72725854) was not biallelic and one (rs17886163) was not present in the European population studied by Conti et al. (2021).

The inventors used Conti et al.'s (2021) European population estimates of the odds ratio (OR) per effect allele and effect allele frequency (p) for each SNP to calculate a population-standardised PRS (Conran et al., 2016; Mealiffe et al., 2010) for each participant. First, the unscaled population average risk (μ) for each SNP was calculated as μ=(1−p)²+2p(1−p)OR+p²OR². Next, for each participant, the adjusted risk (which has a population mean equal to 1) for each SNP was calculated as

$$adjusted risk = \frac{{OR}^{N}}{μ} ,$$

where N is the number of effect alleles. Missing SNPs were given an adjusted risk of 1. Each participant's PRS (as a relative risk) was obtained by multiplying their adjusted risks for each of the SNPs.

Follow-up began at the date of the baseline assessment and finished at the earliest of date of diagnosis of prostate cancer, date of death or 31 Jul. 2019 (the date to which linkage to cancer registries is complete).

The inventors randomly divided the data into a 70% training dataset and a 30% testing dataset that were balanced for affected status. In the training dataset, the inventors fitted Cox proportional hazards models using age as the time axis. The inventors used age as the time axis so that country-specific or ethnicity-specific incidence data can be used with the final Cox model to calculate absolute risk of prostate cancer.

The inventors estimated hazard ratios (HRs) for the risk of prostate cancer for the natural logarithm of the PRS and for the number of affected first-degree relatives (0, 1 or 2). The inventors used Schoenfeld residuals to assess the proportional hazards assumption of the Cox models. Where the proportional hazards assumption was not met, the inventors included the affected risk factors as time-varying covariates. The fit of the final model was visually assessed using a graph of the Nelson-Aalen cumulative hazard function and the Cox-Snell residuals.

For the 5-year absolute risk of prostate cancer, the inventors first calculated participant's relative risk (rrisk) of prostate cancer using the coefficients from the Cox model. The inventors used age-specific (in 5-year groups) and calendar year-specific prostate cancer incidences for England as the population reference rates (Office for National Statistics, 2021) in the calculation of absolute 5-year risks. The inventors used these to determine the population incidence from birth to age b (popincid) and to age b+5 (popincid5) for each participant (where b was his current age in years). The inventors then calculated each participant's cumulative risks to age b years and age b+5 years as: cumul=1−e^{-rrisk×popincid} and cumul5=1−e^{-rrisk×popincid5}, respectively. The inventors used age-specific (in 5-year groups) and calendar year-specific mortality rates for causes of death other than prostate cancer (Office of National Statistics, 2021) for a competing mortality adjustment in the calculation of 5-year absolute risk. For each participant, his expected survival in the next five years was surv5=e^{-mort5}, where mort5 was his expected mortality in the next five years. The 5-year absolute risk of prostate cancer (the risk score) was then calculated as:

$$abs\_risk \_ 5 yr = \frac{( cumul 5 - cumul ) \times surv 5}{( 1 - cumul )} .$$

To assess the performance of the 5-year risk score, the inventors limited follow-up to 5 years and used the approach in MacInnis et al. (2013). The inventors used Cox regression to estimate the HR per standard deviation (in unaffected men) of the log odds of the risk score. The discrimination of the risk score was assessed using Harrell's C-index, excluding men who were unaffected and did not complete 5 years of follow-up. To assess the calibration of the risk score, the inventors used the standardised incidence ratio (SIR) comparing the observed number of cases to the number predicted by the absolute 5-year risk (divided by 5 to obtain a per year risk), overall, by 10-year age group and by quintile of risk. To assess the dispersion of the 5-year risk, the inventors used logistic regression with no constant term where the log odds of the 5-year risk was the dependent variable and the inventors tested whether the coefficient was equal to 1.

Finally, the inventors re-estimated the model to refine the coefficients using whole dataset and calculated SIRs comparing the observed number of cases to the number predicted using population incidence rates, overall, by 10-year age group and by decile of 5-year risk. The inventors used Stata (version 16.1) (Statacorp, 2019) for analyses; all statistical tests were two sided and P values<0.05 were considered nominally statistically significant.

The UK Biobank has Research Tissue Bank approval (REC #11/NW/0382) that covers analysis of data by approved researchers. All participants provided written informed consent to the UK Biobank before data collection began. This research has been conducted using the UK Biobank resource under Application Number 47401.

The data underlying this article was provided by the UK Biobank Bycroft et al., 2018; Sudlow et al., 2015) and the inventors do not have permission to share the data. Researchers wishing to access the data used in this study can apply directly to the UK Biobank at www.ukbiobank.ac.uk/register-apply/. Stata 16.1 (Statacorp, 2019) code for the analysis is available for non-commercial purposes from the corresponding author on request.

### Example 2—Data Set

The 189 338 unaffected men had a mean age of 57.1 (standard deviation [SD]=8.1) years at cohort entry and the 8996 men with incident prostate cancer had a mean age of 62.1 (SD=5.5) years at cohort entry and a mean age of diagnosis of 67.9 (SD=5.8) years. Unaffected men had a mean of 10.2 (SD=1.5) years of follow-up and affected men had a mean of 5.8 (SD=3.0) years of follow-up until their diagnosis.

Overall, 3620 (1.8%) of the participants had all of the 264 available SNPs genotyped, 156 371 (78.8%) were missing 1-5 SNPs, 37 608 (19.0%) were missing 6-10 SNPs and 735 (0.4%) were missing 11 or more SNPs. There was no difference in the distribution of missing SNPs between affected and unaffected men (z=1.72, P=0.09; see Table 8). The mean PRS was 1.02 (SD=0.92) for unaffected men and 1.73 (SD=1.61) for affected men. In the unaffected men, 13 739 (7.3%) had one affected relative and 269 (0.1%) had two affected relatives. In the affected men, 1086 (12.1%) had one affected relative and 43 (0.5%) had two affected relatives.

TABLE 8
Number of single-nucleotide polymorphisms (SNPs)
genotyped, overall and by affected status.

| Number of SNPs | Unaffected | | Affected | | Total | |
|---|---|---|---|---|---|---|
| genotyped | N | % | N | % | N | % |
| 240 | 1 | 0.00 | 0 | 0.00 | 1 | 0.00 |
| 243 | 1 | 0.00 | 0 | 0.00 | 1 | 0.00 |
| 244 | 2 | 0.00 | 0 | 0.00 | 2 | 0.00 |
| 245 | 1 | 0.00 | 0 | 0.00 | 1 | 0.00 |
| 246 | 2 | 0.00 | 0 | 0.00 | 2 | 0.00 |
| 247 | 3 | 0.00 | 0 | 0.00 | 3 | 0.00 |
| 248 | 10 | 0.01 | 1 | 0.01 | 11 | 0.01 |
| 249 | 27 | 0.01 | 1 | 0.01 | 28 | 0.01 |
| 250 | 44 | 0.02 | 0 | 0.00 | 44 | 0.02 |
| 251 | 94 | 0.05 | 2 | 0.02 | 96 | 0.05 |
| 252 | 165 | 0.09 | 2 | 0.02 | 167 | 0.08 |
| 253 | 363 | 0.19 | 16 | 0.18 | 379 | 0.19 |
| 254 | 879 | 0.46 | 40 | 0.44 | 919 | 0.46 |
| 255 | 2060 | 1.09 | 90 | 1.00 | 2150 | 1.08 |
| 256 | 4729 | 2.50 | 227 | 2.52 | 4956 | 2.50 |
| 257 | 9903 | 5.23 | 510 | 5.67 | 10 413 | 5.25 |
| 258 | 18 297 | 9.66 | 873 | 9.70 | 19 170 | 9.67 |
| 259 | 29 078 | 15.36 | 1458 | 16.21 | 30 536 | 15.40 |
| 260 | 37 810 | 19.97 | 1762 | 19.59 | 39 572 | 19.95 |
| 261 | 38 779 | 20.48 | 1777 | 19.75 | 40 556 | 20.45 |
| 262 | 29 229 | 15.44 | 1419 | 15.77 | 30 648 | 15.45 |
| 263 | 14 395 | 7.60 | 664 | 7.38 | 15 059 | 7.59 |
| 264 | 3446 | 1.83 | 154 | 1.71 | 3620 | 1.83 |

Note:
264 of the original list of 269 SNPs were available in the UK Biobank.

When the variables of interest (natural logarithm of the PRS, first-degree family history and age in 10-year groups) were considered together in a Cox regression model in the training dataset, examination of the Schoenfeld residuals showed that there was evidence that the global test of the proportional hazards assumption had not been met (χ²=41.69, degrees of freedom [df]=4, P<0.001). Investigation of the individual variables in the model showed that this arose from the natural logarithm of the PRS (χ²=24.55, df=1, P<0.001) and from having one affected first-degree relative (χ²=11.47, df=1, P<0.001).

The model was therefore re-fitted including those two variables as time-varying covariates (i.e. varying with age, which is the time axis in these analyses). Table 9 shows the HRs, 95% CIs and P values for the final model. The HR for the natural logarithm of the PRS was 6.396 and decreased by a factor of 0.986 for each year of age. Similarly, the HR for having one affected first-degree relative was 6.568 and decreased by a factor of 0.978 for each year of age, and the HR for having two affected first-degree relatives was 139.2 and decreased by a factor of 0.940 for each year of age. Ten-year age group increased risk of prostate cancer with a HR of 1.097. For the final model, the global test of the proportional hazards assumption showed that there was no evidence for a violation (χ²=9.72, df=7, P=0.2). There was, however, evidence the proportional hazards assumption was violated for having an affected first-degree relative (χ²=7.34, df=1, P=0.007) and the interaction between age and having an affected first-degree relative (χ²=7.41, df=1, P=0.007). But, because the proportional hazards test is sensitive to small variations in large datasets, the inventors inspected plots of the scaled Shoenfeld residuals by age for both of these variables and concluded that there was no problem with either variable.

TABLE 9
Cox regression model for risk of prostate cancer in training dataset

| | Hazard | 95% confidence | P |
|---|---|---|---|
| Variable | ratio | interval | value |
| In(PRS) | 6.396 | 4.381, 9.337 | <0.001 |
| Number of affected | | | |
| first-degree relatives | | | |
| 0 | — | | |
| 1 | 6.568 | 2.745, 15.713 | <0.001 |
| 2 | 139.2 | 1.679, 11534 | 0.03 |
| 10-year age group | 1.097 | 1.018, 1.184 | 0.02 |
| Age (years) by In(PRS) | 0.986 | 0.981, 0.992 | <0.001 |
| Age (years) by number | | | |
| of affected first- | | | |
| degree relatives | | | |
| 0 | — | | |
| 1 | 0.978 | 0.966, 0.991 | <0.001 |
| 2 | 0.940 | 0.880, 1.003 | 0.06 |

Note:
In(PRS), natural logarithm of the polygenic risk score.

In the training dataset with follow-up limited to 5 years, the HR per SD of 5-year risk was 2.884 (95% CI=2.671 to 3.114, P<0.001) and the Harrell's C-index was 0.802 (95% CI=0.794 to 0.809).

In the testing dataset with follow-up limited to 5 years, the HR per SD of 5-year risk was 3.058 (95% CI=2.720 to 3.438, P<0.001) and the Harrell's C-index was 0.811 (95% CI=0.800 to 0.821). Table 10 shows that, overall, the 5-year risk score slightly over-estimated risk in the testing dataset with 1088 observed and 1159.09 expected cases of prostate cancer. When stratified by 10-year age group or quintile of risk, the 5-year risk score was well calibrated except for the 40-49 years age group (with 14 observed and 25.6 expected cases of prostate cancer) and the second-lowest quintile of risk (with 34 observed and 58.2 expected cases of prostate cancer).

TABLE 10
Standardised incidence ratios of the number of prostate cancers observed
in the first five years of follow-up in the testing dataset compared
with the number expected using the 5-year risk predictions.

| | | | Standardised | 95% |
|---|---|---|---|---|
| | | | incidence | confidence |
| Variable | Observed | Expected | ratio | interval |
| Overall | 1088 | 1159.1 | 0.939 | 0.885, 0.996 |
| Age group | | | | |
| 40-49 years | 14 | 25.6 | 0.548 | 0.324, 0.925 |
| 50-59 years | 227 | 257.4 | 0.882 | 0.774, 1.004 |
| 60-69 years | 847 | 876.1 | 0.967 | 0.904, 1.034 |
| Quintile of risk | | | | |
| (median risk) | | | | |
| 1 (0.08%) | 11 | 10.5 | 1.046 | 0.579, 1.888 |
| 2 (0.48%) | 34 | 58.2 | 0.584 | 0.417, 0.817 |
| 3 (1.19%) | 144 | 140.4 | 1.026 | 0.871, 1.208 |
| 4 (2.25%) | 267 | 269.1 | 0.992 | 0.880, 1.119 |
| 5 (4.74%) | 632 | 680.8 | 0.928 | 0.859, 1.004 |

### Example 3—Models

Given the similarity of the association and discrimination of the model in the training and testing datasets, and the acceptable calibration of the model in the testing dataset, the inventors re-estimated the model using the full dataset to obtain final risk estimates (see Tables 11 and 12) and used these to calculate the 5-year risk of prostate cancer for all participants.

TABLE 11
Cox regression model for risk of prostate cancer in full dataset.

| | Hazard | 95% confidence | P |
|---|---|---|---|
| Variable | ratio | interval | value |
| In(PRS) | 5.846 | 4.264, 8.013 | <0.001 |
| Number of affected | | | |
| first-degree relatives | | | |
| 0 | — | | |
| 1 | 9.173 | 4.473, 18.81 | <0.001 |
| 2 | 161.4 | 4.272, 6102 | 0.006 |
| 10-year age group | 1.053 | 0.989, 1.121 | 0.1 |
| Age (years) by In(PRS) | 0.987 | 0.983, 0.992 | <0.001 |
| Age (years) by number | | | |
| of affected first- | | | |
| degree relatives | | | |
| 0 | — | | |
| 1 | 0.974 | 0.963, 0.984 | <0.001 |
| 2 | 0.937 | 0.888, 0.989 | 0.02 |

Note:
In(PRS), natural logarithm of the polygenic risk score.

TABLE 12
Cox regression model for risk of prostate cancer in full dataset.

| | | | 95% | |
|---|---|---|---|---|
| | Variable | | confidence | P |
| Variable | name | Coefficient | interval | value |
| In(PRS) | Inprs | 1.766 | 1.450, 2.081 | <0.001 |
| Number of | | | | |
| affected | | | | |
| first-degree | | | | |
| relatives | | | | |
| 0 | fh0 (0 = no, | — | | |
| | 1 = yes) | | | |
| 1 | fh1 (0 = no, | 2.216 | 1.498, 2.934 | <0.001 |
| | 1 = yes) | | | |
| 2 | fh2 (0 = no, | 5.084 | 1.452, 8.716 | 0.006 |
| | 1 = yes) | | | |
| 10-year | agegp | 0.052 | −0.011, 0.115 | 0.1 |
| age group | (1 = 40-40, | | | |
| | 2 = 50-59, | | | |
| | 3 = 60-69) | | | |
| Age (years) | age × Inprs | −0.013 | −0.017, −0.008 | <0.001 |
| by In(PRS) | | | | |
| Age (years) | | | | |
| by number | | | | |
| of affected | | | | |
| first-degree | | | | |
| relatives | | | | |
| 0 | age × fh0 | — | | |
| | (0 = no, 1 = yes) | | | |
| 1 | age × fh1 | −0.027 | −0.037, −0.016 | <0.001 |
| | (0 = no, 1 = yes) | | | |
| 2 | age × fh2 | −0.065 | −0.119, −0.011 | 0.02 |
| | (0 = no, 1 = yes) | | | |

Note:
In(PRS), natural logarithm of the polygenic risk score.

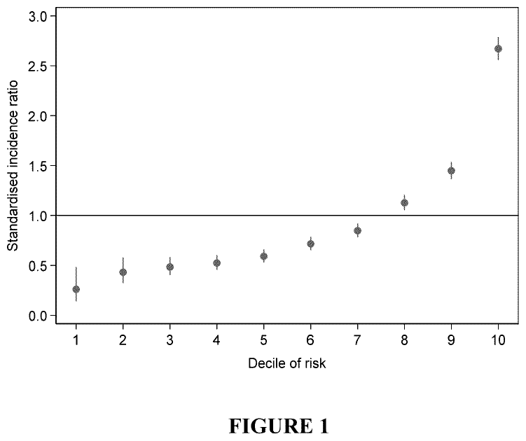

Overall, the number of prostate cancers observed during the first five years of follow-up was higher than the number expected using age-specific (in 5-year groups) and calendar year-specific population incidence rates (3699 and 3012.91, respectively). This was evident in each of the 10-year age groups (see Table 13). Stratification by decile of risk (see Table 13 and FIG. 1) shows that the model can identify the top 10% of men who are at 2.8 times population risk and the next highest 10% of men who are at 1.5 times population risk. At the other end of the risk spectrum, the model can identify men who are at a very low risk of prostate cancer. Men in the lowest three deciles of risk are at about 0.4 times population average risk, and men in the next decile are at about half the population average risk. Risk greater than population average risk is only seen in the top three deciles of risk.

TABLE 13
Standardised incidence ratios of the number of prostate cancers observed
in the first five years of follow-up in the full dataset compared
with the number expected using age-specific (in 5-year groups) and
calendar year-specific population incidence rates, overall, by 10-
year age group and by decile of 5-year risk of prostate cancer.

| | | | Standardised | 95% |
|---|---|---|---|---|
| | | | incidence | confidence |
| Variable | Observed | Expected | ratio | interval |
| Overall | 3699 | 3012.9 | 1.228 | 1.189, 1.268 |
| Age group | | | | |
| 40-49 years | 71 | 59.4 | 1.196 | 0.947, 1.509 |
| 50-59 years | 716 | 614.9 | 1.164 | 1.082, 1.253 |
| 60-69 years | 2912 | 2338.6 | 1.245 | 1.201, 1.291 |
| Decile of risk | | | | |
| (median risk) | | | | |
| 1 (0.03%) | 7 | 18.4 | 0.381 | 0.182, 0.800 |
| 2 (0.13%) | 22 | 56.7 | 0.388 | 0.255, 0.589 |
| 3 (0.32%) | 57 | 135.1 | 0.422 | 0.326, 0.547 |
| 4 (0.58%) | 115 | 231.3 | 0.497 | 0.414, 0.597 |
| 5 (0.90%) | 195 | 317.7 | 0.614 | 0.533, 0.706 |
| 6 (1.28%) | 266 | 378.9 | 0.702 | 0.623, 0.792 |
| 7 (1.74%) | 348 | 422.5 | 0.824 | 0.742, 0.915 |
| 8 (2.36%) | 562 | 458.2 | 1.227 | 1.129, 1.332 |
| 9 (3.36%) | 719 | 483.8 | 1.486 | 1.382, 1.599 |
| 10 (5.80%) | 1408 | 510.4 | 2.759 | 2.759, 2.907 |

In view of investigations by the inventors an individual's relative risk of prostate cancer can be calculated as:

$$rrisk = {e}^{( 1.013 \times lnprs + 0.427 \times fh 1 + 0.837 \times fh 2 + 0.123 \times age 1 + 0.277 \times age 2 + 0.011 \times ( age - 55 ) \times lnprs )}$$

For example, a 57-year-old man with one affected first-degree relative and a PRS of 1.10 (i.e. lnPRS=0.0953) will have:

$$rrisk = {e}^{( 1.013 \times 0.0953 + 0.427 \times 1 + 0.837 \times 0 + 0.123 \times 1 + 0.277 \times 0 + 0.011 \times ( 57 - 55 ) \times 0.0953 )}$$

$$rrisk = {e}^{0.6486}$$

$$rrisk = 1.9129$$

Following which the inventors developed an improved method where an individual's relative risk of prostate cancer can be calculated as:

$$rrisk = {e}^{( 1.766 \times lnprs + 2.216 \times fh 1 + 5.084 \times fh 2 + 0.052 \times agegp - 0.013 \times age \times lnprs - 0.027 \times age \times fh 1 - 0.065 \times age \times fh 2 )}$$

where
lnprs is the natural logarithm of the PRS,
fh1 is 1 if the subject has at least one first degree relative with, or who have had, prostate cancer, and 0 if not,
fh2 is 1 if the subject has two or more first degree relative with, or who have had, prostate cancer, and 0 if not,
agegp1 is a categorical classification of the subject's age in years (1=40-49, 2=50-59, 3=60-69),
age is the age of the subject in years.

For example, a 57-year-old man with one affected first-degree relative and a PRS of 1.10 (i.e. lnprs=0.0953) will have:

$$rrisk = {e}^{( 1.766 \times 0.0953 + 2.216 \times 1 + 5.084 \times 0 + 0.052 \times 2 - 0.013 \times 57 \times 0.0953 - 0.027 \times 57 \times 1 - 0.065 \times 57 \times 0 )}$$

$$rrisk = {e}^{0.8787}$$

$$rrisk = 2.4077$$

The model can be used for many different ethnicities including Caucasians, African ancestry (including African Americans), East Asian ancestry and Hispanic ancestry.

The present application claims priority from AU2022901108 filed 27 Apr. 2022, the entire contents of which are incorporated herein by reference.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Adessi et al. (2000) Nucl. Acids Res. 28, E87.
American Cancer Society. American Cancer Society recommendations for prostate detection. Available at https://www.cancer.org/cancer/prostate-cancer early cancer/detection-diagnosis-staging/acs-recommendations.html (Accessed 31 Jan. 2022).
Barry and Simmons (2017) Medical Clinics of North America, 101:787-806.
Bennett et al. (2005) Pharmacogenomics 6:373-382.
Birren et al. (1997) Genome Analysis: Analyzing DNA, A Laboratory Manual Vol 1.
Brandão et al. (2020) International Journal of Molecular Sciences, 21:5036.
Brenner et al. (2000) Nat. Biotechnol. 18:630-634.
Bycroft et al. (2018) Nature, 562:203-209.
Cabarkapa et al. (2016) Prostate Int, 4:125-129.
Chang et al. (2015) GigaScience; 4: s13742-13015-10047-13748.
Chee et al. (1996) Science 274:610-614.
Conran et al. (2016) Asian Journal of Andrology, 18:520-524.
Conti et al. (2021) Nature Genetics; 53:65-75.
Devlin and Risch (1995) Genomics. 29:311-322.
Fenton et al. (2018) JAMA, 319:1914-1931.
Fodor (1997a) FASEB Journal 11: A879.
Fodor (1997b) Science 277:393-395.
Fry et al. (2017) American Journal of Epidemiology; 186:1026-1034.
Handscomb et al. (2019) PLoS One; 14: e0214311.
Jahn et al. (2015) International Journal of Cancer, 137:2795-2802.
Lockhart (1998) Nature Medicine 4:1235-1236.
MacInnis et al. (2013) British Journal of Cancer, 109:1296-1301.
MacLean et al. (2009) Nature Rev. Microbiol, 7:287-296.
Margulies et al. (2005) Nature 437:376-380.
Mavaddat et al. (2015) J Natl Cancer Inst 107: doi: 10.1093/jnci/djv036.
Mealiffe et al. (2010) Journal of the National Cancer Institute, 102:1618-1627.
Mitra et al. (2003) Analytical Biochemistry 320:55-65.
Morozova and Marra (2008) Genomics 92:255.
National Health Service. PSA testing: prostate cancer. Available at https://www.nhs.uk/conditions/prostate-cancer/psa-testing/(Accessed 31 Jan. 2022).
Office for National Statistics. Cancer registration statistics. Available at www.ons.gov.uk/peoplepopulationandcommunity/healthandsocialcare/conditionsanddi seases/datasets/cancerregistrationstatisticscancerregistrationstatisticsengland (Accessed Jul. 20, 2021).
Office of National Statistics. Mortality statistics-underlying cause, sex and age. Available at www.nomisweb.co.uk/query/construct/summary.asp?mode=construct&version=0&data set=161 (Accessed Jul. 20, 2021).
Office of National Statistics. Mortality statistics. Available at www.nomisweb.co.uk/query/construct/summary.asp?mode=construct&version=0&data set=161 (Accessed Jul. 20, 2021).
Pereira-Azevedo et al. (2017) JMIR Cancer; 3: el.
Pilie et al. (2016) Asian Journal of Andrology; 18:530-532.
Privé et al. (2019) The American Journal of Human Genetics 105:1213-1221.
Public Health England. Prostate cancer risk management programme: overview. Available at https://www.gov.uk/guidance/prostate-cancer-risk-management-programme-overview (Accessed 31 Jan. 2022).
Purcell and Chang. Plink 1.9. Available at https://www.cog-genomics.org/plink/1.9/(Accessed 30 Mar. 2021).
Rawla (2019) World Journal of Oncology, 10:63-89.
Sapolsky et al. (1999) Genet Anal: Biomolec Engin 14:187-192.
Shendure et al. (2005) Science 309:1728-1732.
Shore et al. (2020) Urol Oncol: Semin Orig Investig, 38:683. e11-683. e18.
Slatkin and Excoffier (1996) Heredity 76:377-383.
StataCorp. (2019) Stata statistical software: Release 16. StataCorp LLC: College Station, TX.
Sudlow et al. (2015) PLOS Medicine, 12: e1001779.
UK Biobank. Data providers and dates of data availability. Available at https://biobank.ctsu.ox.ac.uk/showcase/exinfo.cgi?src=Data_providers_and_dates (Accessed 13 Dec. 2021).
Voelkerding et al. (2009) Clinical Chem. 55:641-658.

1. A method for assessing the risk of a human male subject for developing prostate cancer comprising:
i) performing a genetic risk assessment of the subject, wherein the genetic risk assessment involves detecting, in a biological sample derived from the subject, the presence of at least two polymorphisms associated with a risk of a human male subject for developing prostate cancer,
ii) performing a clinical risk assessment of the subject for developing prostate cancer, and
iii) combining the genetic risk assessment and the clinical risk assessment to obtain the risk of a human subject for developing prostate cancer.

2. The method of claim 1, wherein the genetic risk assessment comprises detecting the presence of at least one, at least two, at least five, at least 10, at least 25, at least 50, at least 100, at least 150, at least 200, or at least 250 of the polymorphisms selected from any one of Tables 1 to 4, or a polymorphism in linkage disequilibrium with one or more thereof.

3. The method of claim 1 or claim 2, wherein the genetic risk assessment comprises detecting the presence of each of the polymorphisms provided in Table 1, Table 2, Table 3 or Table 4, or a polymorphism in linkage disequilibrium with one or more thereof.

4. The method of any one of claims 1 to 3, wherein performing the clinical risk assessment involves obtaining information from the subject on one or more of the following: age, ethnicity, family history of prostate cancer incorporating number of first-degree relatives who have had prostate cancer, the age of the youngest first-degree relative at diagnosis with prostate cancer, the number of second-degree relatives who have had prostate cancer, country of residence or protein marker levels.

5. The method of any one of claims 1 to 4, wherein performing the clinical risk assessment comprises obtaining information from the subject on age and first-degree relative history of prostate cancer.

6. The method of any one of claims 1 to 4, wherein performing the clinical risk assessment consists of obtaining information from the subject on age and first-degree relative history of prostate cancer.

7. The method of any one of claims 1 to 6, wherein the subject is at least 40 years old.

8. The method of any one of claims 1 to 6, wherein the subject is between 40 and 69 years old.

9. The method of any one of claims 1 to 8, wherein the results of the risk assessment indicate that the subject should be enrolled in a screening program or subjected to more frequent screening.

10. The method of any one of claims 2 to 9, wherein the polymorphism in linkage disequilibrium has linkage disequilibrium above 0.9.

11. The method of any one of claims 2 to 10, wherein the polymorphism in linkage disequilibrium has linkage disequilibrium of 1.

12. The method of any one of claims 1 to 11 which further comprises comparing the risk to a pre-determined threshold.

13. The method of any one of claims 1 to 12, wherein the genetic risk assessment produces a polygenic risk score (PRS).

14. The method of claim 13, wherein the polygenic risk score is determined using an odds ratio (OR) for each effect allele and effect allele frequency (p).

15. The method of claim 14, wherein for each polymorphism the unscaled population average risk (μ) is calculated as:
$$μ = {( 1 - p )}^{2} + 2 p ( 1 - p ) OR + {p}^{2} {OR}^{2} .$$

16. The method of claim 15, wherein an adjusted risk for each polymorphism is calculated as adjusted risk=(OR^{N})/μ, where N is the number of effect alleles.

17. The method of claim 16, wherein the polygenic risk score is determined by combining the adjusted risk for each polymorphism.

18. The method of claim 17, wherein the adjusted risk for each polymorphism are combined by multiplication.

19. The method according to any one of claims 13 to 18 which comprises determining the natural logarithm of the PRS (lnPRS).

20. The method of claim 19, wherein lnPRS is multiplied by a predetermined β coefficient.

21. The method of claim 20, wherein the β coefficient is about 1.7 such as 1.766.

22. The method according to any one of claims 13 to 21 which comprises determining the relative risk (rrisk) of developing prostate cancer using;
$$rrisk = {e}^{( PDCE 1 \times lnprs + PDCE 2 \times fh 1 + PDCE 3 \times fh 2 + PDCE 4 \times agegp + PDCE 5 \times age \times lnprs + PDCE 6 \times age \times fh 1 + PDCE 7 \times age \times fh 2 )}$$
PDCE1 is a predetermined β coefficient for the natural logarithm of a PRS,
PDCE2 is a predetermined β coefficient if the subject has at least one first-degree relative with, or who has had, prostate cancer,
PDCE3 is a predetermined β coefficient if the subject has two or more first-degree relatives with, or who have had, prostate cancer,
PDCE4 is a predetermined β coefficient for age category,
PDCE5 is a predetermined β coefficient for the interaction between age in years and the PRS,
PDCE6 is a predetermined β coefficient based on the interaction between age in years and if the subject has at least one first-degree relative with, or who has had, prostate cancer,
PDCE7 is a predetermined β coefficient based on the interaction between age in years and if the subject has two or more first-degree relatives with, or who have had, prostate cancer,
lnprs is the natural logarithm of the PRS,
fh1 is 1 if the subject has at least one first-degree relative with, or who has had, prostate cancer, and 0 if not,
fh2 is 1 if the subject has two or more first-degree relatives with, or who have had, prostate cancer, and 0 if not,
age is the age of the subject in years.

23. The method of claim 22, wherein the age categories are 40 to 49, 50 to 59 and 60 to 69.

24. The method of claim 23, wherein
i) if the subject is 40 to 49 years of age the value is 1,
ii) if the subject is 50 to 59 years of age the value is 2, and
iii) if the subject is 60 to 69 years of age the value is 3.

25. The method according to any one of claims 22 to 24, wherein one or more or all of the following apply;
a) PDCE1 is between 1.266 and 2.266,
b) PDCE2 is between 1.716 and 2.716,
c) PDCE3 is between 4.084 and 6.084,
d) PDCE4 is between 0.022 and 0.082,
e) PDCE5 is between −0.003 and −0.023,
f) PDCE6 is between −0.017 and −0.037,
f) PDCE7 is between −0.035 and −0.095.

26. The method according to any one of claims 22 to 25, wherein one or more or all of the following apply;
a) PDCE1 is 1.766,
b) PDCE2 is 2.216,
c) PDCE3 is 5.084,
d) PDCE4 is 0.052,
e) PDCE5 is −0.013,
f) PDCE6 is −0.027,
f) PDCE7 is −0.065.

27. The method of claim 20, wherein the β coefficient is about 1.03 such as 1.0307.

28. The method according to any one of claim 13 to 20 or 27 which comprises determining the relative risk (rrisk) of developing prostate cancer using;
$$rrisk = {e}^{( PDCE 1 \times lnprs + PDCE 2 \times fh 1 + PDCE 3 \times fh 2 + PDCE 4 \times age 1 + PDCE 5 \times age 2 + PDCE 6 \times ( age - 55 ) \times lnprs )}$$
where;
PDCE1 is a predetermined β coefficient for the natural logarithm of a PRS,
PDCE2 is a predetermined β coefficient if the subject has at least one first-degree relative with, or who has had, prostate cancer,
PDCE3 is a predetermined β coefficient if the subject has two or more first-degree relatives with, or who have had, prostate cancer,
PDCE4 is a predetermined β coefficient if the subject is 50 to 59 years of age,
PDCE5 is a predetermined β coefficient if the subject is 60 to 69 years of age,
PDCE6 is a predetermined β coefficient based on an interaction between the age of the subject and the natural logarithm of the PRS,
lnprs is the natural logarithm of the PRS
fh1 is 1 if the subject has at least one first-degree relative with, or who has had, prostate cancer, and 0 if not,
fh2 is 1 if the subject has two or more first-degree relatives with, or who have had, prostate cancer, and 0 if not,
age1 is 1 if the subject is 50 to 59 years of age and 0 if not,
age2 is 1 if the subject is 60 to 69 years of age and 0 if not,
age is the age of the subject in years.

29. The method of claim 28, wherein one or more or all of the following apply;
a) PDCE1 is between 0.913 and 1.113,
b) PDCE2 is between 0.327 and 0.527,
c) PDCE3 is between 0.737 and 0.937,
d) PDCE4 is between 0.023 and 0.223,
e) PDCE5 is between 0.177 and 0.377,
f) PDCE6 is between −0.089 and 0.111.

30. The method of claim 28 or claim 29, wherein one or more or all of the following apply;
a) PDCE1 is 1.013,
b) PDCE2 is 0.427,
c) PDCE3 is 0.837,
d) PDCE4 is 0.123,
e) PDCE5 is 0.277,
f) PDCE6 is 0.011.

31. The method according to any one of claims 1 to 30 which comprises determining one or more or all of the absolute 5-year risk, the absolute 10-year risk, or the absolute remaining lifetime risk (to age 90).

32. A computer-implemented method for assessing the risk of a human male subject for developing prostate cancer, the method operable in a computing system comprising a processor and a memory, the method comprising:
receiving clinical risk data and genetic risk data for the male subject, wherein the clinical and genetic risk data was obtained by a method according to any one of claims 1 to 31;
processing the data to combine the clinical risk data with the genetic risk data to obtain the risk of a human male subject for developing prostate cancer;
outputting the risk of a human male subject for developing prostate cancer.

33. The computer-implemented method of claim 32, wherein the clinical risk data and genetic risk data for the subject is received from a user interface coupled to the computing system.

34. The computer-implemented method of claim 32 or claim 33, wherein the clinical risk data and genetic risk data for the subject is received from a remote device across a wireless communications network.

35. The computer-implemented method of any one of claims 32 to 34, wherein outputting comprises outputting information to a user interface coupled to the computing system.

36. The computer-implemented method of any one of claims 32 to 35 which comprises determining a genetic risk score based on genetic data derived from a biological sample taken from the male subject.

37. The computer-implemented method of any one of claims 32 to 36 which comprises determining the relative risk (rrisk) of developing prostate cancer using;
$$rrisk = {e}^{( PDCE 1 \times lnprs + PDCE 2 \times fh 1 + PDCE 3 \times fh 2 + PDCE 4 \times agegp + PDCE 5 \times age \times lnprs + PDCE 6 \times age \times fh 1 + PDCE 7 \times age \times fh 2 )}$$
PDCE1 is a predetermined β coefficient for the natural logarithm of a PRS,
PDCE2 is a predetermined β coefficient if the subject has at least one first-degree relative with, or who has had, prostate cancer,
PDCE3 is a predetermined β coefficient if the subject has two or more first-degree relatives with, or who have had, prostate cancer,
PDCE4 is a predetermined β coefficient for age category,
PDCE5 is a predetermined β coefficient for the interaction between age in years and the PRS,
PDCE6 is a predetermined β coefficient based on the interaction between age in years and if the subject has at least one first-degree relative with, or who has had, prostate cancer,
PDCE7 is a predetermined β coefficient based on the interaction between age in years and if the subject has two or more first-degree relatives with, or who have had, prostate cancer,
lnprs is the natural logarithm of the PRS,
fh1 is 1 if the subject has at least one first-degree relative with, or who has had, prostate cancer, and 0 if not,
fh2 is 1 if the subject has two or more first-degree relatives with, or who have had, prostate cancer, and 0 if not,
age is the age of the subject in years.

38. The computer-implemented method of any one of claims 32 to 36 which comprises determining the relative risk (rrisk) of developing prostate cancer using;
$$rrisk = {e}^{( PDCE 1 \times lnprs + PDCE 2 \times fh 1 + PDCE 3 \times fh 2 + PDCE 4 \times age 1 + PDCE 5 \times age 2 + PDCE 6 \times ( age - 55 ) \times lnprs )}$$
where;
PDCE1 is a predetermined β coefficient for the natural logarithm of a PRS,
PDCE2 is a predetermined β coefficient if the subject has at least one first-degree relative with, or who has had, prostate cancer,
PDCE3 is a predetermined β coefficient if the subject has two or more first-degree relatives with, or who have had, prostate cancer,
PDCE4 is a predetermined β coefficient if the subject is 50 to 59 years of age,
PDCE5 is a predetermined β coefficient if the subject is 60 to 69 years of age,
PDCE6 is a predetermined β coefficient based on an interaction between the age of the subject and the natural logarithm of the PRS,
lnprs is the natural logarithm of the PRS, fh1 is 1 if the subject has at least one first degree relative with, or who have had, prostate cancer, and 0 if not,
fh2 is 1 if the subject has two or more first degree relative with, or who have had, prostate cancer, and 0 if not,
age1 is 1 if the subject is 50 to 59 years of age and 0 if not,
age2 is 1 if the subject is 60 to 69 years of age and 0 if not,
age is the age of the subject in years.

39. A computer readable storage medium storing executable code, wherein when a processor executes the code, the processor is cause to perform the method of any one of claims 32 to 38.

40. A device for assessing the risk of a human male subject developing prostate cancer, the device comprising:
a processor; and
a memory device storing executable code, the memory being accessible to the processor;
wherein when caused to execute the executable code stored in the memory device, the processor is caused to perform a method according to any one of claims 1 to 39.

41. The device of claim 40 further comprising a display component, wherein the processor is further caused to display the prostate cancer risk score of the male subject for developing prostate cancer on the display component.

42. The device of claim 40 or claim 41 further comprising a communications module, wherein the processor is further caused to communicate the prostate cancer risk score of the male subject for developing prostate cancer to an external device via the communications module.

43. A method for determining the need for routine diagnostic testing of a human male subject for prostate cancer comprising assessing the risk of the subject for developing prostate cancer using the method according to any one of claims 1 to 39.

44. A method of screening for prostate cancer in a human male subject, the method comprising assessing the risk of the subject for developing prostate cancer using the method according to any one of claims 1 to 39, and routinely screening for prostate cancer in the subject if they are assessed as having a risk for developing prostate cancer.

45. A method for determining the need of a human male subject for prophylactic anti-prostate cancer therapy comprising assessing the risk of the subject for developing prostate cancer using the method according to any one of claims 1 to 39.

46. A method for preventing prostate cancer in a human male subject, the method comprising assessing the risk of the subject for developing prostate cancer using the method according to any one of claims 1 to 39, and administering an anti-prostate cancer therapy to the subject if they are assessed as having a risk for developing prostate cancer.

47. An anti-prostate cancer therapy for use in preventing prostate cancer in a human male subject at risk thereof, wherein the subject is assessed as having a risk for developing prostate cancer according to the method of any one of claims 1 to 39.

48. A method for stratifying a group of human male subjects for a clinical trial of a candidate therapy, the method comprising assessing the individual risk of the subjects for developing prostate cancer using the method according to any one of claims 1 to 39, and using the results of the assessment to select subjects more likely to be responsive to the therapy.
